(19) United States
(12) Patent Application Publication
(10) Pub. No.: US 2025/0082656 A1
(43) Pub. Date: Mar. 13, 2025

(54) DOSAGE REGIMENS FOR TREATMENT OF DENGUE INFECTION

(71) Applicant: Atea Pharmaceuticals, Inc., Boston, MA (US)

(72) Inventors: Adel Moussa, Burlington, MA (US); Narayan C. Chaudhuri, Acton, MA (US); Steven Good, Leland, NC (US); Kai Lin, Belmont, MA (US); Xiao-Jian Zhou, Arlington, MA (US); Jean-Pierre Sommadossi, Boston, MA (US); Kejiang Hu, Waltham, MA (US); Robert S. Lewis, Waltham, MA (US); Bruno Canard, Paris (FR); Ashleigh Shannon, Paris (FR)

(73) Assignee: Atea Pharmaceuticals, Inc., Boston, MA (US)

(21) Appl. No.: 18/957,362

(22) Filed: Nov. 22, 2024

Related U.S. Application Data
(63) Continuation of application No. PCT/US2023/023739, filed on May 26, 2023.
(60) Provisional application No. 63/346,274, filed on May 26, 2022.
(60) Provisional application No. 63/414,812, filed on Oct. 10, 2022.
(60) Provisional application No. 63/427,359, filed on Nov. 22, 2022.

(51) Int. Cl. A61K 31/688; A61K 9/20; A61K 31/685; A61K 31/708; A61P 31/14

(52) U.S. Cl. CPC A61K 31/688; A61K 9/2013; A61K 9/2054; A61K 31/685; A61K 31/708; A61P 31/14

(57) ABSTRACT

The present invention provides advantageous high-dose therapeutic compositions and combinations to treat or prevent Dengue virus infections that may be serotype 1, 2, 3 or 4 as well as a process of manufacture.

### CROSS-REFERENCE TO RELATED APPLICATIONS

This application is a continuation of International Patent Application No. PCT/US2023/023739, filed in the U.S. Receiving Office on May 26, 2023, which claims the benefit of U.S. Provisional Application No. 63/346,274 filed May 26, 2022; U.S. Provisional Application No. 63/414,812 filed Oct. 10, 2022; and U.S. Provisional Application No. 63/427,359 filed Nov. 22, 2022. The entirety of each of these applications is hereby incorporated by reference for all purposes.

### FIELD OF THE INVENTION

The present invention provides advantageous therapeutic compositions, combinations and uses thereof for the treatment or prophylaxis of Dengue virus infection.

### BACKGROUND OF THE INVENTION

Dengue fever is caused by four related viruses, known as Dengue virus serotypes 1-4. Dengue viruses (DENV) are members of the Flavivirus genus of the Flaviviridae family. They are enveloped viruses with an 11 kb positive-sense RNA genome which encodes ten proteins. These proteins are the membrane (M) protein, envelope (E) protein, capsid (C) protein, and non-structural (NS) proteins. The NS proteins are NS1, NS2A, NS2B, NS3, NS4A, NS5B and NS5. These proteins are translated as a single polyprotein which is cleaved by proteases into the individual proteins. The NS5 protein is the largest of the non-structural proteins, and acts as the viral RNA dependent RNA polymerase as well as an RNA 2′-O-methyltransferase. The RNA-dependent RNA polymerase (RdRp) activity is responsible for replication of the viral genome.

Dengue is endemic to the tropics, including Eastern Mediterranean, the Americas, South-East Asia, Western Pacific, and Africa. As the range of the Aedes mosquitos which carry Dengue has grown, new cases have been observed in areas where it previously had never been seen. The United States Centers for Disease Control and Prevention has reported recent outbreaks of locally acquired Dengue in Hawaii (2015), Florida (2013, 2020) and Texas (2013) (https://www.cdc.gov/Dengue/areaswithrisk/in-the-us.html). The mosquitos that carry Dengue are now common across much of the southeast United States (Rivera et al. 2020. Travel-associated and Locally Acquired Dengue Cases—United States, 2010-2017. MMWR Morb Mortal Wkly Rep 69:149-154).

Although the distribution of individual Dengue serotypes was previously limited geographically, as the disease has spread around the world the distribution of serotypes has become more pervasive (Nature Scitable “Dengue Viruses”). This is particularly concerning because if a person is infected with one serotype and then later becomes infected with a second, they are at greater risk of severe Dengue. Severe Dengue (or Dengue hemorrhagic fever) is accompanied by severe plasma leakage, severe bleeding, shock and organ impairment. If untreated, severe Dengue has a mortality rate of 13% (Centers for Disease Control and Prevention, Dengue for Healthcare Providers, Clinical Presentation, updated Apr. 13, 2023).

In addition to the geographic spread of Dengue outbreaks, recent outbreaks have been more severe. Worldwide, the incidence has increased 8-fold since the year 2000. Not only has the frequency of outbreaks increased, but also the magnitude. The World Health Organization estimates that half the world's population is at risk of infection, and hundreds of millions of infections are recorded each year (https://www.who.int/news-room/fact-sheets/detail/Dengue-and-severe-Dengue). Nearly half a million cases of severe Dengue are also reported. The majority of those at risk of getting Dengue are children (Aranda C et al., 2018. Arbovirus surveillance: first Dengue virus detection in local Aedes albopictus mosquitoes in Europe, Catalonia, Spain, 2015. Euro Surveill 23, Wilder-Smith et al., 2014. The 2012 Dengue outbreak in Madeira: exploring the origins. Euro Surveill 19:20718).

Vaccines against Dengue have been developed but suffer from several drawbacks. The most significant is a phenomenon known as antibody dependent enhancement (ADE). The only licensed vaccine, Dengvaxia, offers incomplete (https://www.fda.gov/news-events/press-announcements/first-fda-approved-vaccine-prevention-dengue-disease-endemic-regions) and uneven protection against different serotypes, with only 50% protection against DENV1, and less for DENV2 (Sabchareon et al. 2012. Protective efficacy of the recombinant, live-attenuated, CYD tetravalent Dengue vaccine in Thai school children: a randomized, controlled phase 2b trial. Lancet 380:1559-67; Villar et al., 2015. Efficacy of a tetravalent Dengue vaccine in children in Latin America. N Engl J Med. 372:113-23). Vaccination can therefore promote ADE, and consequently severe infection in Dengue naive individuals (Aguiar et al., 2016. The Impact of Newly Licensed Dengue Vaccine in Endemic Countries. PLoS Negl Trop Dis. 10:e0005179). Thus, it is restricted to individuals 9-16 years of age who have had at least one documented previous Dengue virus infection. Despite the current and growing significance of Dengue virus as a worldwide threat to health, the development of a safe and effective pan-Dengue vaccine has yet to be achieved.

Thus, a potential strategy to combat Dengue worldwide is pharmaceuticals, for example, a direct acting antiviral (DAA). Unfortunately, there are no approved DAAs for Dengue fever. In DENV, the viral polymerase is the nonstructural protein NS5 (Karuna et al., 2020. A Cyclic Phosphoramidate Prodrug of 2′-Deoxy-2′-Fluoro-2′C-Methylguanosine for the Treatment of Dengue Virus Infection. Antimicrob Agents Chemother 64). Although the DENV RdRp has been the target of many investigational DAAs, including many nucleoside/nucleotide analogues (Troost et al., 2020. Recent advances in antiviral drug development towards Dengue virus. Curr Opin Virol 43:9-21), to date only one nucleoside, the cytosine analog balapiravir has been tested clinically (Karuna et al., 2020. A Cyclic Phosphoramidate Prodrug of 2′-Deoxy-2′-Fluoro-2′C-Methylguanosine for the Treatment of Dengue Virus Infection. Antimicrob Agents Chemother 64; Nguyen et al., 2013. A randomized, double-blind placebo-controlled trial of balapiravir, a polymerase inhibitor, in adult Dengue patients. J Infect Dis. 207:1442-50). Unfortunately, no differences between compound and placebo treatment were observed regarding antiviral response, cytokine profile and time to fever clearance (Nguyen et al., 2013. A randomized, double-blind placebo-controlled trial of balapiravir, a polymerase inhibitor, in adult Dengue patients. J Infect Dis. 207:1442-50).

Other compounds which target NS5 or other viral proteins have been reported in the literature. In 2017, Janssen disclosed a series of compounds which bind to the NS3 protein (WO 2017/167951). In 2020 the Manfroni lab described pyridobenzothiazolones which inhibit NS5 (Cannalire, R. et al. Pyridobenzothiazolones Exert Potent Anti-Dengue Activity by Hampering Multiple Functions of NS5 Polymerase, ACS Med Chem Lett, 2020 11, 773-782). Other publications describing Dengue inhibitors include but are not limited to Moquin et al. NITD-688, a pan-serotype inhibitor of the Dengue virus NS4B protein, shows favorable pharmacokinetics and efficacy in preclinical animal models. Science Translational Medicine, 2021, 13, 579; Kaptein, S. et al. A pan-serotype Dengue virus inhibitor targeting the NS3-NS4B interaction. Nature, 2021, 598, 504-509; Shimizu, H. et al. Discovery of a small molecule inhibitor targeting Dengue virus NS5 RNA-dependent RNA polymerase. PLoS Negl Trop Dis. 2019, 13, e0007894; Yin, Z. et al. An adenosine nucleoside inhibitor of Dengue virus, PNAS, 2009, 106, 20435-20439; Putra, H. et al. Identification of natural product compounds as NS5 RDRP inhibitor for Dengue virus serotype 1-4 through in silico analysis. AIP Conference Proceedings 2020, 2237; Wang, Q.-Y. et al. A Small-Molecule Dengue Virus Entry Inhibitor. Antimicrobial Agents and Chemotherapy, 2009, 53, 1823-1831; de Oliviera, L. et al. The small molecule AZD6244 inhibits Dengue virus replication in vitro and protects against lethal challenges in a mouse model. 2020, Archives of Virology, doi:10.1007/s00705-020-04524-7; and Celegato, M. et al. Small-Molecule Inhibitor of Flaviviral NS3-NS5 Interaction with Broad-Spectrum Activity and Efficacy In Vivo, Antimicrobial Chemotherapy, 2022 14(1) e03097-22.

Specific antiviral compounds that treat certain RNA viruses include Compound 1 and Compound 2. The free base, Compound 1, is disclosed, for example, in U.S. Pat. No. 9,828,410.

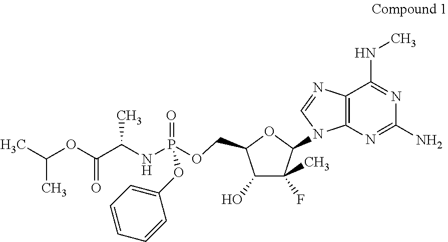 [SMILES: CNc1nc(N)nc2c1ncn2[C@@H]1O[C@H](CO[P@](=O)(N[C@@H](C)C(=O)OC(C)C)Oc2ccccc2)[C@@H](O)[C@@]1(C)F]

Compound 2, disclosed in U.S. Pat. No. 10,519,186, is the hemi-sulfate salt of isopropyl ((R)-(((2R,3R,4R,5R)-5-(2-amino-6-(methylamino)-9H-purin-9-yl)-4-fluoro-3-hydroxy-4-methyltetrahydrofuran-2-yl)methoxy)(phenoxy)phosphoryl)-L-alaninate and is disclosed in particular for the treatment of a viral infection such as hepatitis C virus. Compound 2 has also been reported as active against SARS-CoV2, the causative agent of COVID19 infection (see for example U.S. Pat. No. 10,874,687). The S-phosphorus diastereomer of Compound 2 is currently in human clinical trials for the treatment of HCV and COVID19.

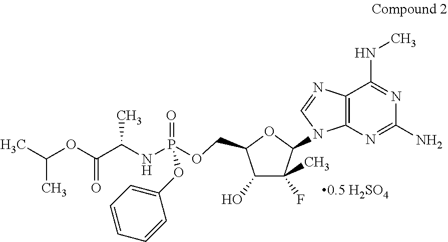 [SMILES: CNc1nc(N)nc2c1ncn2[C@@H]1O[C@H](CO[P@](=O)(N[C@@H](C)C(=O)OC(C)C)Oc2ccccc2)[C@@H](O)[C@@]1(C)F]

In U.S. Pat. No. 10,946,033 Atea Pharmaceuticals, Inc. disclosed the use of Compound 1 or Compound 2 generally for the treatment of Flaviviruses, with activity data against yellow fever, Dengue (serotype 2), West Nile virus, and Zika virus.

Despite these disclosures, it has proved difficult to effectively treat Dengue due to the challenging issues described above. There remains a strong medical need to determine the proper protocol for the treatment of Dengue that is safe, effective, and well-tolerated.

It is therefore an object of the present invention to provide advantageous regimes to treat infections of Dengue. It is a further object of the present invention to provide new methods of manufacture of Compound 1 and Compound 2, which is the phosphorus R-diastereomer.

### SUMMARY OF THE INVENTION

It has been unexpectedly discovered that Compound 1 or Compound 2 (and wherein Compound 1 can be a pharmaceutically acceptable salt thereof) in a high dose (i.e., at least about 700 mg or 750 mg, and more generally 700-1000 mg per dose (which can be delivered in one, two or three separate dosage forms for convenience) two, three or more times a day) is advantageous for the treatment of Dengue virus, when administered to a host, such as a human, in need thereof. In typical embodiments, Compound 1 or Compound 2 is provided in one or more pharmaceutical compositions to reach this effective amount

Further, it has been discovered that Compound 1 or Compound 2 are also active against Dengue serotypes 1, 3 and 4. With this new discovery, it is confirmed that Compound 1 and Compound 2 in the high dose regimens described herein possesses potency against all serotypes of Dengue virus (Dengue virus serotype 1, 2, 3, and 4).

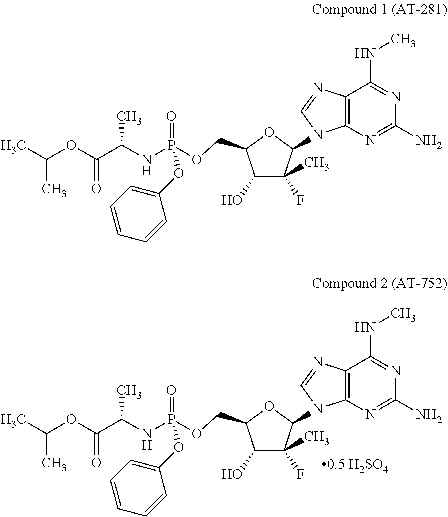 [SMILES: CNc1nc(N)nc2c1ncn2[C@@H]1O[C@H](CO[P@](=O)(N[C@@H](C)C(=O)OC(C)C)Oc2ccccc2)[C@@H](O)[C@@]1(C)F ; CNc1nc(N)nc2c1ncn2[C@@H]1O[C@H](CO[P@](=O)(N[C@@H](C)C(=O)OC(C)C)Oc2ccccc2)[C@@H](O)[C@@]1(C)F]

The term “Compound 1” as used in the specification can refer to either the free base or a pharmaceutically acceptable salt thereof. Compound 1 is isopropyl ((R)-(((2R,3R,4R,5R)-5-(2-amino-6-(methylamino)-9H-purin-9-yl)-4-fluoro-3-hydroxy-4-methyltetrahydrofuran-2-yl) methoxy)(phenoxy)phosphoryl)-L-alaninate.

It has also been discovered that Compound 1 (and its hemisulfate salt Compound 2) have an advantageous dual mechanism of action. The active metabolite is an inhibitor of the RNA dependent RNA polymerase (RdRp) domain of the viral protein NS5 as well as an inhibitor of the 2′-O-methyltransferase (MTase) domain of NS5. Both functions of the NS5 protein are required for replication. Inhibition of two key protein functions involved in RNA replication decreases the chance of a resistant mutant forming.

As discussed in Example 2 and shown in Table 1 and FIG. 2, it has been discovered that Compound 1 (AT-281) exhibits in vitro potency against all four serotypes of Dengue including serotype 1 (EC₅₀=0.57 μM), serotype 2 (EC₅₀<7 μM in multiple strains), serotype 3 (EC₅₀<1.5 μM in multiple strains), and serotype 4 (EC₅₀<1.2 μM in multiple strains). The pan-serotype activity is advantageous because it minimizes the risk of severe Dengue and lessens or eliminates the need for serotype testing prior to administration of the compound.

Alternatively, Compound 1 can be used in the free base form or as any desired pharmaceutically acceptable salt in the high dose regimen described herein. Compound 2, the hemisulfate salt of Compound 1, is an advantageous form of Compound 1 with improved in vivo properties. For example, Compound 2 is effective in vivo in a mouse model of Dengue infection (D2Y98P virus strain) as described in Example 18. The use of Compound 2, for example, provides a significant difference in viremia observed on days 6 and 8 post infection (pi) in the blood shown in FIG. 8A and in the spleen shown in FIG. 8B. Additionally, significant effects on prevention of weight loss shown in FIG. 9A and deterioration of health score shown in FIG. 9B on Days 3 through 8 pi were seen relative to vehicle control treated animals. Importantly, there was a highly significant prolongation of survival, shown in FIG. 10, which attests to the efficacy of Compound 2 against Dengue virus, or in the alternative Compound 1 as a free base or a pharmaceutically acceptable salt thereof.

In certain aspects Compound 2 or Compound 1 (as a free base or a pharmaceutically acceptable salt thereof) is administered to a patient, typically in the high-dose regimen described herein, wherein the patient has multiple flaviviruses and wherein one of the flaviviruses is Dengue, for example, a patient with Dengue serotype 1, 2, 3, or 4 and another flavivirus. Compound 1 (AT-281) has exhibited potent effects in reducing viral titer against 11 different strains of 7 flaviviruses tested in vitro in 3 different cell types with a range of concentrations to achieve 50% inhibition of the virus-induced cytopathic effect (CPE) of 0.21 to 1.41 μM (Table 1 and FIG. 2).

As described in Example 10 and shown in FIG. 1, the active metabolite of Compound 2 or Compound 1 (Compound 5) acts as a GTP analog and is incorporated into RNA by the NS5 RNA dependent RNA polymerase (RdRp), immediately terminating synthesis.

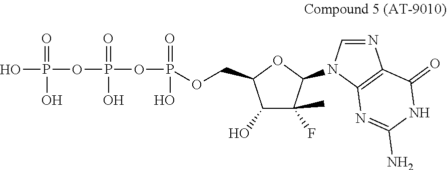 [SMILES: C[C@@]1(F)[C@H](O)[C@@H](COP(=O)(O)OP(=O)(O)OP(=O)(O)O)O[C@H]1n1cnc2c(=O)[nH]c(N)nc21]

The chain-terminating efficiency of Compound 5 was measured in competitive nucleotide incorporation assays using all four DENV serotypes. Compound 5 is incorporated into RNA 9 to 12-fold less compared to native substrate guanosine triphosphate (i.e., for every approximately 7-8 GTPs incorporated, one AT-9010 will be incorporated). In comparison, sofosbuvir triphosphate is incorporated into the RNA less than 1 time for every 100 UTP (native uridine triphosphate) incorporations. The discrimination assay data is provided below.

| Preference | DENV1 | DENV2 | DENV3 | DENV4 |
|---|---|---|---|---|
| GTP > AT-9010 | 7.7 ± 0.5 | 9.3 ± 0.1 | 11.6 ± 1.3 | 11.2 ± 0.7 |
| GTP > AT-9002 | 19.4 ± 2.8 | 18.2 ± 0.2 | 17.5 ± 1.5 | 41.5 ± 7.3 |
| UTP > SOF-TP | >100 | >100 | >100 | >100 |

Compound 5 (and thus Compound 1 and Compound 2) is also an inhibitor of the 2′-O-methyltransferase (MTase) domain of Dengue virus NS5. The MTase domain is responsible for capping the 2′-end of the replicated viral RNA. The RNA cap mitigates host immune response to the viral RNA. Inhibition of the MTase domain is a second mechanism of action for Compound 5.

The binding of Compound 5 to the MTase domain was measured in a thermal shift assay. Compound 5 stabilized DENV1, DENV2, DENV3, and DENV4 MTase domain nearly two fold greater than the previously reported MTase inhibitor sinefungin (1.9±0.1° C. for sinefungin compared to 3.8±0.1° C. for Compound 5).

Furthermore, Compound 5 more effectively stabilized the RdRp domain than native substrate guanosine triphosphate (5.6±0.1° C. for Compound 5 compared to 4.5±0.2° C. for GTP), whereas sinefungin negligibly stabilized the RdRp domain (0.1° C.±0.1° C.).

Confirming the activity of Compound 5 against the MTase domain, an IC₅₀ of 29.6+2 μM was measured in a filter-binding assay. As it has been previously shown that Compound 5 reaches hundreds of micromolar concentration in cells (Good, S. et al. Preclinical evaluation of AT-527, a novel guanosine nucleotide prodrug with potent, pan-genotypic activity against hepatitis C virus, PLoS ONE 15(1): e0227104), an IC₅₀ of 29.6 μM indicates that Compound 5 is an effective inhibitor of the MTase domain.

Compound 5, the active metabolite of Compound 2 and Compound 1, thus has multiple mechanisms of action against Dengue virus. Multiple mechanisms of action may prevent the emergence of resistance, especially against the highly conserved MTase domain.

In certain aspects of the present invention, a compound of Formula I:

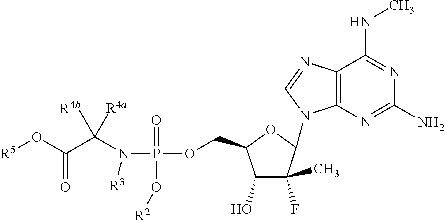

or a pharmaceutically acceptable salt thereof optionally in a pharmaceutically acceptable carrier, is administered in a high dose (at least 700 or 750 mg per dose, for example 700-1000 mg per dose (which can be provide in 1, 2, 3 or 4 separate dosage forms for convenience of swallowing), two, three or more times a day) to treat a Dengue virus infection in a host in need thereof, typically a human;
wherein:
R² is C₁₋₆alkyl (including methyl, ethyl, propyl, and isopropyl), C₃₋₇cycloalkyl, aryl, aryl(C₁-C₄alkyl)-, heteroaryl, heteroaryl(C₁-C₄alkyl)-, or heteroalkyl, each of which is optionally substituted with 1, 2, 3, or 4 substituents independently selected at each occurrence from R²²;
R³ is hydrogen or C₁₋₆alkyl optionally substituted with 1, 2, 3, or 4, substituents independently selected at each occurrence from R³³;
R^{4a} and R^{4b} are independently selected from hydrogen, C₁₋₆alkyl, aryl(C₁₋₆alkyl)-, C₃₋₇cycloalkyl, C₃₋₇cycloalkyl(C₁₋₆alkyl)-, heteroaryl(C₁₋₆alkyl)-, and aryl, each of which is optionally substituted with 1, 2, 3, or 4 substituents independently selected at each occurrence from R⁴⁴;
R⁵ is hydrogen, C₁₋₆alkyl (including methyl, ethyl, propyl, and isopropyl), C₁₋₆haloalkyl, or C₃₋₇cycloalkyl and in an alternative embodiment, R⁵ is aryl(C₁-C₄alkyl)-, aryl, heteroaryl, or heteroalkyl, optionally substituted with 1, 2, 3, or 4 substituents independently selected at each occurrence from R⁵⁵.

R²², R³³, R⁴⁴, and R⁵⁵ are independently selected at each occurrence from hydrogen, C₁-C₆alkyl, C₃-C₇cycloalkyl, alkenyl, alkynyl, F, Cl, Br, I, heterocycle, heterocycloalkyl, haloalkoxy, haloalkyl, C(O)R⁶⁶, C(O)OR⁶⁶, C(O)N(R⁶⁶)₂, and cyano;
R⁶⁶ is independently selected at each occurrence from hydrogen, C₁-C₆alkyl, aryl(C₁-C₆alkyl)-, C₃-C₇cycloalkyl, C₃-C₇cycloalkyl(C₁-C₆alkyl)-, heteroaryl(C₁-C₆alkyl)-, and aryl.

In certain aspects of the present invention, a compound of Formula II:

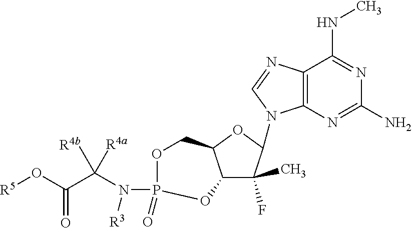

or a pharmaceutically acceptable salt thereof optionally in a pharmaceutically acceptable carrier, is administered in a high dose (at least 700-1000, for example, 700-850 mg per dose two, three or more times a day) to treat a Dengue virus infection in a host in need thereof, typically a human;
wherein:
R³ is hydrogen or C₁₋₆alkyl optionally substituted with 1, 2, 3, or 4, substituents independently selected at each occurrence from R³³;
   R^{4a} and R^{4b} are independently selected from hydrogen, C₁₋₆alkyl, aryl(C₁₋₆alkyl)-, C₃₋₇cycloalkyl, C₃₋₇cycloalkyl(C₁₋₆alkyl)-, heteroaryl(C₁₋₆alkyl)-, and aryl, each of which is optionally substituted with 1, 2, 3, or 4 substituents independently selected at each occurrence from R⁴⁴;
   R⁵ is hydrogen, C₁₋₆alkyl (including methyl, ethyl, propyl, and isopropyl), C₁₋₆haloalkyl, or C₃₋₇cycloalkyl and in an alternative embodiment, R⁵ is aryl(C₁-C₄alkyl)-, aryl, heteroaryl, or heteroalkyl, optionally substituted with 1, 2, 3, or 4 substituents independently selected at each occurrence from R⁵⁵.
   R²², R³³, R⁴⁴, and R⁵⁵ are independently selected at each occurrence from hydrogen, C₁-C₆alkyl, C₃-C₇cycloalkyl, alkenyl, alkynyl, F, Cl, Br, I, heterocycle, heterocycloalkyl, haloalkoxy, haloalkyl, C(O)R⁶⁶, C(O)OR⁶⁶, C(O)N(R⁶⁶)₂, and cyano;
   R⁶⁶ is independently selected at each occurrence from hydrogen, C₁-C₆alkyl, aryl(C₁-C₆alkyl)-, C₃-C₇cycloalkyl, C₃-C₇cycloalkyl(C₁-C₆alkyl)-, heteroaryl(C₁-C₆alkyl)-, and aryl.

All R groups are intended to be interpreted in a manner that does not include redundancy i.e., as known in the art, alkyl would not be substituted with alkyl; however, for example, alkoxy substituted with alkoxy is not redundant. Aryl substituted with aryl falls within the definition of aryl in a limited scope. R groups are not optionally substituted unless specifically indicated in context.

Non-limiting examples of C₁-C₆alkyl include methyl, ethyl, propyl, isopropyl, butyl, t-butyl, sec-butyl, isobutyl, —CH₂C(CH₃)₃, —CH(CH₂CH₃)₂, and —CH₂CH(CH₂CH₃)₂. Non-limiting examples of C₃-C₆cycloalkyl include cyclopropyl, CH₂-cyclopropyl, cyclobutyl, and CH₂-cyclobutyl.

A non-limiting example of aryl(C₁-C₄alkyl)- is benzyl. Non-limiting examples of aryl are phenyl and naphthyl.

In certain aspects, the compound is of the Formula IIA:

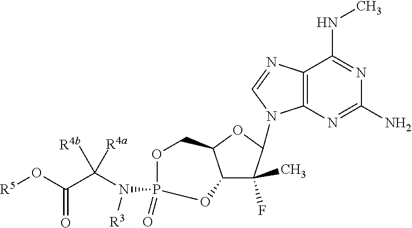

or a pharmaceutically acceptable salt thereof, wherein all variables are as defined herein.

In certain aspects, the compound is of the Formula IIB:

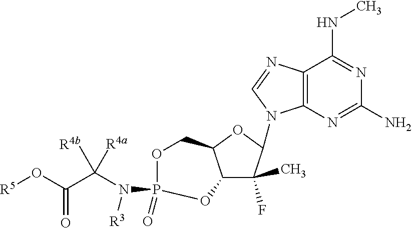

or a pharmaceutically acceptable salt thereof, wherein all variables are as defined herein.

In certain aspects, a selected compound described herein is used to treat Dengue virus infection, which can be serotype 1, 3 and/or 4. In some embodiments, a selected compound described herein is used to treat Dengue virus infection, which may be serotype 1, 2, 3 and/or 4, by administering a dosage of about 700-1000 mg, for example, 700-850 mg per dose two, three or more times a day, (and in some embodiments, 650, 675, 700, 725, 750, 775, 800, 825, 850, 875, 900, 925, 950, 975, or 1000 milligrams two or three times a day) to a patient in need thereof. In certain embodiments, Compound 1 or Compound 2 is administered in a dosage of about 700-1000 mg, for example at least about 700, 750 or 800 mg per dosing (which may be delivered in multiple solid dosage forms to reach the full dose).

In a specific embodiment, Compound 2 is delivered at a dosage of three 250 mg solid dosage forms of free base weight to reach a 750 mg dosage total dosage form twice a day. In other embodiments, Compound 2 is delivered in two 375 mg solid dosage forms to reach a 750 mg dosage total dosage form twice a day.

In certain embodiments Compound 1 or Compound 2 is administered twice, three times or four times a day for four, five, six, seven, eight, nine, or ten days. In certain embodiments this treatment regimen can treat Dengue virus serotypes 1, 3 and/or 4, or even 1, 2 3, and/or 4. For clarity, unless otherwise stated, the milligram dose of active compound administered is based on the weight of the salt of the nucleotide phosphoramidate, if a pharmaceutically acceptable salt is used. For conversion purposes, a dose of about 750 mg of Compound 2 corresponds to a dose of about 692 mg of Compound 1 (which falls within “about 700 mg”), in the free base form.

The present invention includes both treatment and prophylactic or preventative therapies. In some embodiments, the active NS5 RdRp inhibitory compound as described herein is administered as a prophylactic to a host such as a human according to the high dose methods provided herein who has been exposed to and is thus at risk of contracting a Dengue virus infection such as Dengue 1 virus, Dengue 2 virus, Dengue 3 virus, and/or Dengue 4 virus. In another alternative embodiment, a method to prevent transmission is provided that includes administering a high-dose effective amount as described herein of one of the compounds described herein to a human for a sufficient length of time prior to exposure to crowds that can be infected, including during travel or public events or meetings, including for example, up to 3, 5, 7, 10, 12, 14 or more days prior to a communicable situation.

The present invention also provides a stereoselective process for a scalable manufacture of Compound 1 wherein the R_{P} diastereomer is produced in substantially pure form. A substantially pure form of the diastereomer typically refers to at least about 90% or greater of the R_{P} diastereomer over the S_{P} diastereomer. Reaction of a compound of the formula Intermediate A, Intermediate B, and a uronium-based activator, for example 1-[bis(dimethylamino)methylene]-1H-1,2,3-triazolo[4,5-b]pyridinium 3-oxide hexafluorophosphate (HATU), preferentially forms the S_{P} phosphoramidate Intermediate C. A similar process was described in WO 2022/040473A1, assigned to Atea Pharmaceuticals, where the desired product was the S_{P} phosphoramidate.

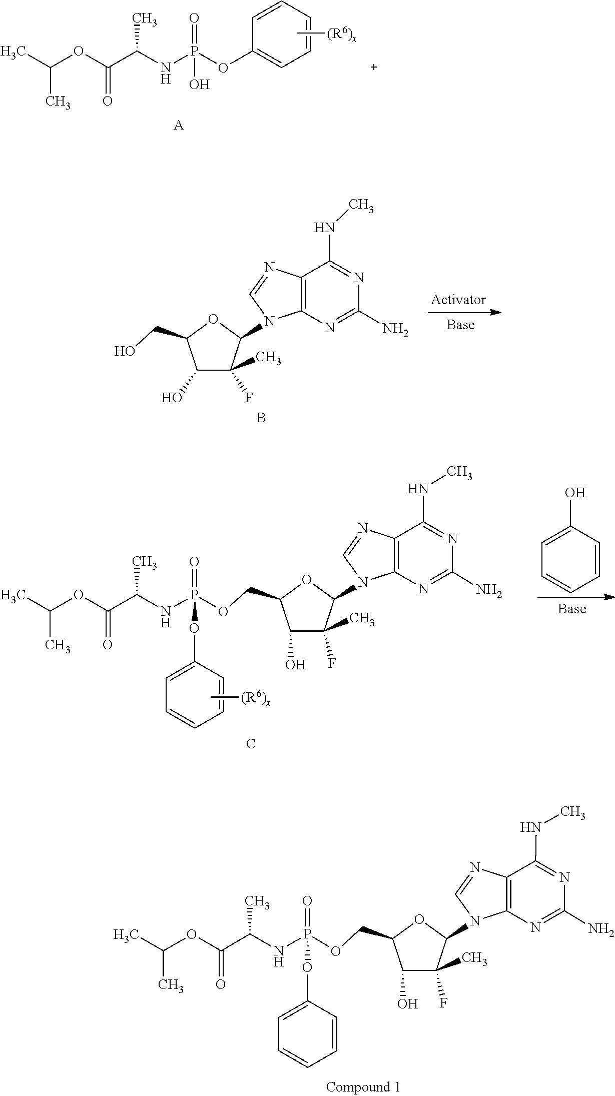

The aryloxy group on the phosphoramidate of Intermediate C is substituted with one, two, or three R⁶ groups which activate it for displacement, for example, by electron withdrawing function. In a second step, the activated phenoxy leaving group is displaced by phenol, inverting the phosphorus stereochemistry to form Compound 1.

In certain embodiments, the substantially pure R-phosphorus diastereomer is about 93% pure or greater, about 95% pure or greater, about 98% pure or greater or even 99% pure or greater.

In certain embodiments the 700 mg to 1,000 mg dose is administered in three solid dosage forms to a patient in need thereof for the treatment of Dengue, wherein the solid dosage forms totaling about 700 mg to about 1,000 mg of Compound 1 or 2 is administered two times a day (a total of six pills daily). In certain embodiments the three solid dosage forms totaling about 700 mg to about 1,000 mg of Compound 1 or 2 are administered four, five, six, or seven days in a row. In certain embodiments three solid dosage forms totaling about 700 mg to about 1,000 mg of Compound 1 or 2 are administered eight, nine, ten, eleven, twelve, thirteen, or fourteen days in a row.

In certain embodiments Composition B is administered four, five, six, or seven days in a row. In certain embodiments Composition A is administered eight, nine, ten, eleven, twelve, thirteen, or fourteen days in a row.

In some embodiments, Compound 1 or a pharmaceutically acceptable salt thereof is used in Composition A or Composition B.

In certain embodiments Compound 1 or Compound 2 is administered two times a day in a dose of least about 675, 700, 725, 750, 775, 800, 825, 850, 875, 900, 925, 950, 975, or 1000 mg wherein the compound is administered, for illustrative example, every 8+/−2 hours.

In certain aspects a dosage regimen described herein is administered within four, three, two, or one day of symptom onset. In certain embodiments a dosage regimen described herein is administered within 48 hours of symptom onset.

The present invention thus includes the following features:
(a) A dosage regimen of Compound 1 or Compound 2 comprising administering at least about 675-1000 or 700-1000 mg per dose, such as 675, 700, 725, 750, 775, 800, 825, 850, 875, 900, 925, 950, 975, or 1000 mg of Compound 1 or Compound 2 twice or three times a day to a patient with Dengue fever serotype 1, 2, 3, and/or 4.
(b) The treatment of Dengue serotype 1, 3 or 4 as further described herein.
(c) A dosage regimen of Compound 1 or Compound 2 comprising administering at least about 675-1000 mg per dose, such as 675, 700, 725, 750, 775, 800, 825, 850, 875, 900, 925, 950, 975, or 1000 mg of Compound 1 or Compound 2 twice or three times a day to a patient with Dengue fever serotype 1, 3, and/or 4.
(d) The dosage regimen of (a), (b) or (c), wherein Compound 1 or Compound 2 is administered on three, four, five, six, seven, eight, nine, ten, eleven, twelve, thirteen, fourteen, or fifteen consecutive days.
(e) The dosage regimen of (a), (b) or (c) wherein Compound 1 or Compound 2 is administered at a dose of about 750 mg three times a day for fourteen days.
(f) The dosage regimen of (a), (b) or (c) wherein Compound 1 or Compound 2 is administered at a dose of about 750 mg three times a day by weight of the free base for five days.
(g) The dosage regimen of (a) or (b), wherein Compound 1 or Compound 2 is administered at a dose of about 1,000 mg by weight of free base two times a day for five, six, or seven days.
(h) The dosage regimen of any one of (a)-(g) wherein Compound 2 is administered to the patient.
(i) The dosage regimen of (h), wherein the dose of about 675 mg to 1,000 mg is administered in three solid dosage forms
(j) The dosage regimen of any one of (a)-(g) wherein Compound 1 is administered to the patient.
(k) A method of treating Dengue in a patient in need thereof comprising administering Compound 1 or Compound 2 in a dosage regimen of any one of (a)-(j).
(l) The method of (k) wherein the dosage regimen comprises administering a pharmaceutical composition comprising Compound 1 or Compound 2.
(m) Use of dosage regimen of any one of (a)-(k) in the manufacture of a medicament for treatment of Dengue fever.
(n) The use of (l) or (m) wherein the dosage regimen comprises administering a pharmaceutical composition comprising Compound 1 or Compound 2.
(o) A method of treating Dengue in a patient in need thereof comprising administering a compound of Formula I according to a high dose protocol described herein.
(p) A method of treating Dengue in a patient in need thereof comprising administering a compound of Formula II, Formula IIA or Formula IIB according to a high dose protocol described herein.
(q) A process for the manufacture of Compound 1 or 2 that includes reacting a compound of the formula Intermediate A, Intermediate B, and a uronium-based activator, for example 1-[bis(dimethylamino)methylene]-1H-1,2,3-triazolo[4,5-b]pyridinium 3-oxide hexafluorophosphate (HATU), in a manner that preferentially forms the S_{P} phosphoramidate Intermediate C, as described above, and then in a second step, the activated phenoxy leaving group is displaced by phenol, inverting the phosphorus stereochemistry to form Compound 1 in the R-phosphorus diasteromeric configuration.
(r) The process of (q) wherein the substantially pure R-phosphorus diastereomer is about 93% pure or greater, about 95% pure or greater, about 98% pure or greater or even 99% pure or greater.

### BRIEF DESCRIPTION OF THE FIGURES

FIG. 1 illustrates Compound 2 (AT-752) and a metabolic pathway to the pharmacologically active metabolite Compound 5 (AT-9010).

FIG. 2 shows concentration dependent inhibition curves of Compound 1 (AT-281) against Dengue-2 virus (DENV), West Nile Virus (WNV), and Yellow Fever virus (YFV). The x-axis is the log concentration of Compound 1 (AT-281) and the y-axis measures the reduction in viral yield as measured by % viral titer. The experimental procedure is provided in Example 2.

FIG. 3 shows the in vitro formation of Compound 5 (AT-9010) in human peripheral blood mononuclear cells. PBMCs were incubated with 10 μM Compound 1 (AT-281) for 6 h. The prodrug was then removed, and the cells rinsed and incubated for another 28 h (washout phase). At each timepoint, cells (n=3) were collected in ice-cold 60% MeOH, and analyzed for Compound 5 (AT-9010), the active intracellular TP, using an LC-MS/MS assay. Data are expressed as mean+/−SD. The x-axis shows the timepoints measured in hours and the y-axis shows the amount of Compound 5 (AT-9010) measured in picomoles/10⁶ cells. The experimental procedure is provided in Example 8.

FIG. 4 shows a phosphorimage of a 20% acrylamide:bisacrylamide (19:1), 7M urea sequencing gel that indicates the results of a nucleotide incorporation assay looking at the incorporation of Compound 5 (AT-9010) in the presence and absence of GTP. The left half of the gel shows the time course of elongation of P₁₀ primer along a T₂₀ RNA template by the DENV-2 NS5 protein with ATP, UTP and CTP (100 μM each) and increasing concentrations of Compound 5 (AT-9010) (0-250 μM). The right half of the gel shows elongation in the presence of all four NTPs (100 μM) in competition with Compound 5 (AT-9010) (100-625 μM). The Compound 5 (AT-9010) incorporation products at +5 and +7 position are shown with the lower dot, while incorporation of the native GTP at the same positions are shown with the upper dot. The experimental procedure is provided in Example 10.

FIG. 5 shows a phosphorimage of a 20% acrylamide:bisacrylamide (19:1), 7M urea sequencing gel that shows the results of a nucleotide incorporation assay looking at the incorporation of the chain terminator 3′dGTP in the presence and absence of GTP. The left half of the gel shows the time course of elongation of P₁₀ primer along a T₂₀ RNA template by the DENV-2 NS5 protein with ATP, UTP and CTP (100 μM each) and increasing concentrations of 3′dGTP (0-250 μM). The right half of the gel shows elongation in the presence of all four NTPs (100 μM) in competition with 3′dGTP (100-625 μM). The 3′dGTP incorporation products at +5 and +7 position are shown with the lower dot, while incorporation of the native GTP at the same positions are shown with the upper dot. The experimental procedure is provided in Example 10.

FIG. 6A shows a line graph measuring the plasma concentration of Compound 1 (AT-281) and its metabolites in mice after oral doses of Compound 2 (AT-752). Male CD-1 mice were administered 420 mg/kg Compound 2 (AT-752). Blood samples were collected up to 24 hrs. post dose, plasma separated and analyzed for Compound 1 (AT-281), Compound 3 (AT-551), and Compound 6 (AT-273) by LC-MS/MS. Data are express as mean+/−SD (n=3-5/time point). The x-axis is time post the dose measured in hours and the y-axis is the plasma concentration of the metabolites measured in nanomoles/mL). The experimental procedure is provided in Example 14.

FIG. 6B shows a line graph measuring the plasma concentration of Compound 1 (AT-281) and its metabolites in rats after oral doses of Compound 2 (AT-752). Male SD rats were administered 300 mg/kg Compound 2 (AT-752). Blood samples were collected up to 24 hrs. post dose, plasma separated and analyzed for Compound 1 (AT-281), Compound 3 (AT-551), and Compound 6 (AT-273) by LC-MS/MS. Data are express as mean+/−SD (n=3-5/time point). The x-axis is time post the dose measured in hours and the y-axis is the plasma concentration of the metabolites measured in nanomoles/mL). The experimental procedure is provided in Example 14.

FIG. 6C shows a line graph measuring the plasma concentration of Compound 1 (AT-281) and its metabolites in monkeys after oral doses of AT-752. Male cynomolgus monkeys were administered 300 mg/kg Compound 2 (AT-752). Blood samples were collected up to 24 hrs. post dose, plasma separated and analyzed for Compound 1 (AT-281), Compound 3 (AT-551), and Compound 6 (AT-273) by LC-MS/MS. Data are express as mean+/−SD (n=3-5/time point). The x-axis is time post the dose measured in hours and the y-axis is the plasma concentration of the metabolites measured in nanomoles/mL). The experimental procedure is provided in Example 14.

FIG. 7 shows the results of in vivo formation of Compound 5 (AT-9010) in PBMCs after oral administration of Compound 2 (AT-752) in mice, rats, and monkeys. Mice and rats were administered an oral dose of Compound 1 (AT-281) and blood collected at 4 and 12 h (mice) and at 24, 48 and 72 h (rats). Monkeys were given an oral dose of Compound 2 (AT-752) equivalent to Compound 1 (AT-281) at 55 mg/kg and blood collected at 12, 24 and 48 h. PBMCs were isolated and Compound 5 (AT-9010) was extracted and quantitated using LC/MS/MS. Mouse PBMCs were pooled from 3 animals per time point; Rat and monkey data expressed as mean+/−SD (n=2-3 per time point). The x-axis is time post dose measured in hours and the y-axis is the concentration of Compound 5 (AT-9010) in PBMCs measured in picomoles/10⁶ cells. The experimental procedure is provided in Example 15.

FIG. 8A shows viremia in DENV-infected mice treated with Compound 2 (AT-752). AG129 mice challenged with DENV2 were treated orally with vehicle (control) or Compound 2 (AT-752) (a 1000 mg/kg loading dose 4 h prior to challenge, followed by BID doses of 500 mg/kg for 7 consecutive days, starting 1 h post inoculation). Blood samples were collected on Days 4, 6, 7, 8 and 10 post infection, and assessed for viral load by a plaque assay. The dotted line=limit of detection; data are expressed as mean+/−SD. *p<0.01 by t-test at each time point. The x-axis represents days post infection measured in days and the y-axis is the amount of viremia measured in blood measured by log₁₀ PFU/mL. The experimental procedure is provided in Example 18.

FIG. 8B shows spleen viral load in DENV-infected mice treated with Compound 2 (AT-752). AG129 mice challenged with DENV2 were treated orally with vehicle (control) or Compound 2 (AT-752) (a 1000 mg/kg loading dose 4 h prior to challenge, followed by BID doses of 500 mg/kg for 7 consecutive days, starting 1 h post inoculation). Spleen samples were collected on Days 4, 6, 7, 8 and 10 post infection, and assessed for viral load by a plaque assay. The dotted line=limit of detection; data are expressed as mean+/−SD. *p<0.01 by t-test at each time point. The x-axis represents days post infection measured in days and the y-axis is the amount of virus in the spleen measured by log₁₀ PFU/gram. The experimental procedure is provided in Example 18.

FIG. 9A shows the weight change of DENV-infected mice treated with Compound 2 (AT-752). AG129 mice challenged with DENV2 were orally administered Vehicle (control) or Compound 2 (AT-752) (a loading dose of 1000 mg/kg 4 h prior to challenge, followed by BID doses of 500 mg/kg for 7 consecutive days starting 1 h post inoculation). Body weights were recorded daily for the duration of the study. Weight was compared to Day 0 (baseline) and presented as percent change. Data expressed as mean+/−SD. *p<0.01 by t-test at each time point. The x-axis represents days post infection measured in days and the y-axis is the weight changes measured in percentage from baseline. The experimental procedure is provided in Example 18.

FIG. 9B shows the health scores of DENV-infected mice treated with Compound 2 (AT-752). AG129 mice challenged with DENV2 were orally administered Vehicle (control) or Compound 2 (AT-752) (a loading dose of 1000 mg/kg 4 h prior to challenge, followed by BID doses of 500 mg/kg for 7 consecutive days starting 1 h post inoculation). Health scores were recorded daily for the duration of the study. Data expressed as mean+/−SD. *p<0.01 by t-test at each time point. The x-axis represents days post infection measured in days and the y-axis is the health score measured numerically with a lower score representing better health. The experimental procedure is provided in Example 18.

FIG. 10 shows a Kaplan-Meier survival curves of DENV-infected mice treated with Compound 2 (AT-752). AG129 mice challenged with DENV2 were administered Vehicle (control) or Compound 2 (AT-752) (a loading dose of 1000 mg/kg 4 h prior to challenge, followed by BID doses of 500 mg/kg for 7 consecutive days starting 1 h post inoculation). Percent survival was calculated up to 20 days post infection, excluding mice sacrificed as scheduled for collection of blood and spleen samples. The treated group was significantly different from control, p<0.001 by the Mantel-Cox log-rank test. The x-axis represents days post infection measured in days and the y-axis is the percent survival. The experimental procedure is provided in Example 18.

FIG. 11 is a line graph showing the plasma concentration of Compound 1 established by 750 mg three times a day dosing of Compound 2. The plasma concentration is maintained above the EC₉₀ of Compound 1 against Dengue serotype 2 (approximately 200 ng/mL) throughout the treatment cycle. Six healthy participants received 13 consecutive oral doses of 750 mg of Compound 2 every 8 hours (three time a day for 4 days and a dose on day 5). Plasma samples for intensive pharmacokinetic evaluation were obtained pre-dose and over 8 hours post the first and eleventh doses, and over 120 hours after the last (thirteenth dose). Plasma samples for trough concentrations were obtained before each dose. Plasma levels of compound 6, considered a surrogate for the intracellular active triphosphate metabolite Compound 5 (FIG. 1) were quantitated using a validated HPLC-MS/MS method.

FIG. 12 is a pharmacokinetic plot showing the concentration of Compound 1 in plasma over time. The dosages tested are 250 mg, 500 mg, 500 mg fed, 1000 mg, 1000 mg in patients from East/Southeast/South Asian origin, and 1500 mg. Plasma samples were taken at pre-determined timepoints and quantified using LC-MS/MS. Analysis of the data can be found in Example 24, Table 10. Plasma exposure of Compound 1 increased over the studied dose range in a greater than dose proportional manner. The high-fat/high-calorie meal delayed the Cₘₐₓ of Compound 1 but had limited to no impact on the total exposure. In fasting patients, Compound 1 was rapidly absorbed and cleared.

FIG. 13 is a pharmacokinetic plot showing the concentration of Compound 3 in plasma over time. The dosages tested are 250 mg, 500 mg, 500 mg fed, 1000 mg, 1000 mg in patients from East/Southeast/South Asian origin, and 1500 mg. Plasma samples were taken at pre-determined timepoints and quantified using LC-MS/MS. Analysis of the data can be found in Example 24, Table 11. Similar to Compound 1, plasma exposure of Compound 3 increased in a greater than dose-proportional manner.

FIG. 14 is a pharmacokinetic plot showing the concentration of Compound 6 in plasma over time. The dosages tested are 250 mg, 500 mg, 500 mg fed, 1000 mg, 1000 mg in patients from East/Southeast/South Asian origin, and 1500 mg. Plasma samples were taken at pre-determined timepoints and quantified using LC-MS/MS. Analysis of the data can be found in Example 24, Table 12. Plasma exposure of Compound 6 increased in a less than dose proportional manner over the ranges studied. Plasma levels of Compound 6 increased slowly and exhibited a long elimination half-life (cohort mean 23 hours). The high-fat/high-calorie meal slightly increased the exposure of Compound 6. Renal clearance of Compound 6 was observed to exceed glomerular filtration rate, suggesting active secretion processes are involved in the renal clearance of this metabolite.

FIG. 15 is a pharmacokinetic plot showing the concentration of Compound 7 in plasma over time. The dosages tested are 250 mg, 500 mg, 500 mg fed, 1000 mg, 1000 mg in patients from East/Southeast/South Asian origin, and 1500 mg. Plasma samples were taken at pre-determined timepoints and quantified using LC-MS/MS. Analysis of the data can be found in Example 24, Table 13. Across the studied dosage range, plasma exposure of Compound 7 increased in a dose dependent manner. Compound 7 exhibited a slower elimination phase than Compounds 2 or 3. Renal clearance for Compound 7 was also observed to be higher than glomerular filtration rate, suggesting active secretion processes are involved in the renal clearance of this metabolite.

FIG. 16. Shows the in vitro activity of AT-281 against DENV1-4 reporters, as described in Example 26. Serial dilutions of AT-281 and virus modified with a nano luciferase reporter (MOIs of 0.1 for DENV-1, DENV-2 and DENV-3; MOI of 0.001 for DENV-4) were added to 96-well plates seeded with 1×104 Huh-7 cells per well. After incubation for 48 h, cells were washed and EC50 values were determined by luciferase activities. Data were normalized to DMSO treatment samples which were set to 100%. Error bars represent mean±SD. Results are representative of three independent experiments with each one analyzed in triplicate.

FIG. 17 shows thermal stability shift experiments showing the stabilizing effect of GTP, AT-9010 or Sinefungin on the DENV serotype 1 to 4 MTases, RdRp and NS5 protein. The proteins were expressed and purified as described in Examples 27-30. The thermal shift assay was performed as described in Example 31. values are reported in delta Tm (ΔTm (° C.)) using the proteins alone as a baseline.

FIG. 18 shows functional and structural basis of inhibition of the DENV MTase by AT-9010. A) Dose-response determination using a filter binding assay (FBA). Reactions were incubated at 30° C. during 30 min and the enzymatic activity was determined using FBA as described in Example 33. B) Overall structure of DENV3 MTase represented in blue ribbons complexed with SAH and AT-9010 represented as sticks. AT-9010 is located at the Cap binding site. C) Omit map at 1σ of the AT-9010. D) Close view of the AT-9010 binding in the GTP binding site. Interacting residues are labelled and shown as sticks. E) Structural comparison of the position of a GTP (pink) and AT9010 (light brown) bound in the Cap binding site. Cap binding site structures belong to DENV2 (in pink—PDB 2P1D)) and DENV3 (in light brown) MTases and are conserved.

FIG. 19A shows AT-9010 incorporation and inhibition during de novo initiation reaction by DENV2 NS5, as described in Example 34. A) PAGE analysis of de novo initiation reactions using a 10-nt template corresponding to the 3′ end of the genome (AACAGGUUCU) resulting in the formation of pppApG, pppApGpA and pppApGpApA over time. GTP was either replaced by AT9010 or AT9010 was added in increasing concentrations to the reactions containing 100 μM GTP. B) Inhibition of de novo product formation by AT-9010 based on the quantification of product bands of four (Mn) or two (Mg) independent reactions; the corresponding IC50 values are given.

FIG. 19B shows the discrimination of AT-9010, AT-9002 (2′-methyl-GTP), and SOF-TP by DENV4 NS5 against their natural nucleotide competitors GTP, GTP, and UTP, respectively. A) Primer extension assay, as described in Example 34, allowing multiple insertion of ATP, CTP, and UTP in the absence, the presence of GTP; or the presence of a mixture of GTP and AT-9010 as indicated below the auto-fluorograph of the gel. The nature of regular inserted Watson-Crick nucleotides is indicated in the right of the gel. B) Primer extension assay allowing incorporation of a single nucleotide GTP, AT-9010, AT-9002, UTP or SOF-TP. Quantification of band-products greater in size than the G/AT (+5) band relative to the G/AT (+5) band were measured using a Typhoon FluorImager, and allowed to calculate the discrimination value. The incorporation rates for AT-901 and AT9002 are compared to that of GTP, and to that of UTP in the case of SOF-TP. Similar results for other DENV1-3 NS5 s are reported in FIGS. 21A-D.

FIG. 20A compares the RdRp active sites of HCV, DENV, and SARS-CoV-2 RdRps. The HCV NS5b cocrystal with sofosbuvir (PDB: 4WTG) was superimposed onto DENV2 NS5 (PDB: 5ZQK). Procedures for the modeling study are described in Example 35. Amino acid side chains in the vicinity of motif D and as well as the nucleotide phosphates are represented. The motif D region is shown for HCV. Metal A and B are represented with spheres in the vicinity of the motif A and C catalytic aspartates, respectively.

FIG. 20B The SARS-CoV-2 nsp12 structure and AT-9010 was superimposed onto DENV NS5 (PDB: 5ZQK) as described in Example 35. The motif D Lysine amino acid side chain is shown for SARS-CoV-2 nsp12 and for DENV NS5, respectively. Essential amino acids contacting the nucleotide phosphates are represented. A unique Metal B is represented with a sphere in the vicinity of the motif A and C catalytic aspartates, respectively (Shannon, A. et al. A second type of N7-guanine RNA cap methyltransferase in an unusual locus of a large RNA virus genome, Nucleic Acids Research, 2022, 50(19) 11186-11198).

FIGS. 21A, 21B, 21C, and 21D show the primer extension assay allowing incorporation of a single nucleotide GTP, AT-9010, AT-9002 (2′-methyl-GTP), UTP, or SOF-TP as described in Example 34, but reported comparatively for the 4 serotypes of DENV NS5. Measurements and quantitations were as in FIG. 19B. The incorporation rates for AT-9010 and 2′-methylguanosine triphosphate (AT-9002) are compared to that of GTP, and to that of UTP in the case of SOF-TP.

FIG. 22 shows the primer extension assay allowing incorporation of multiple nucleotide AT-9010 with either all four NTPs or UTP, ATP, and CTP only as described in Example 34, but reported comparatively for the 4 serotypes of DENV NS5.

FIG. 23 shows the quantification of the gels shown in FIG. 21, and graphic representation of the incorporation rates. The data provided in the graphs can be found in FIG. 21 and the procedure used to collect the data can be found in Example 34. The incorporation rates for AT-9010 and AT9002 are compared to that of GTP, and to that of UTP in the case of SOF-TP.

FIG. 24 shows the structures of Compound 1 and Compound 2.

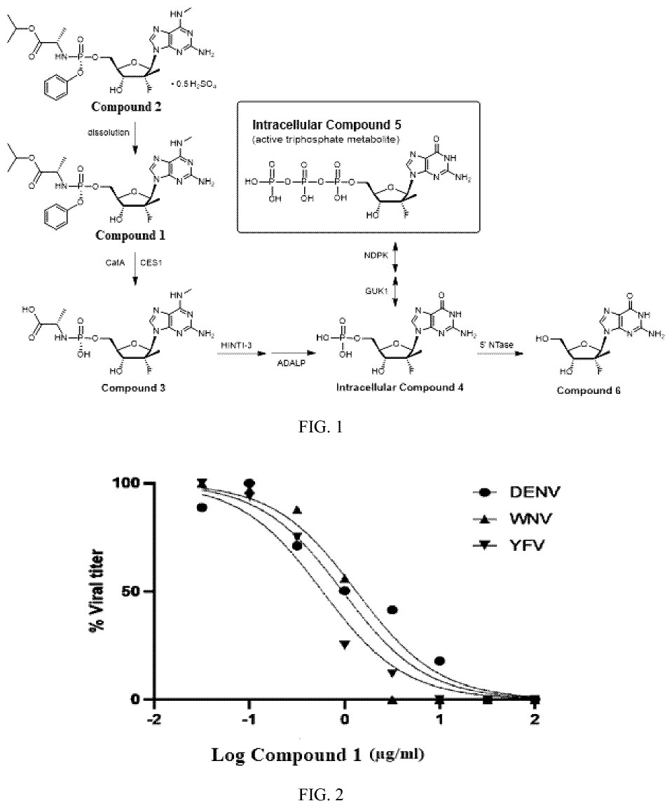

### DETAILED DESCRIPTION OF THE INVENTION

It has been unexpectedly discovered that Compound 1 or Compound 2 (and wherein Compound 1 can be a pharmaceutically acceptable salt thereof) in a high dose (i.e., at least 700-1,000 mg per dose two, three or more times a day) is advantageous for the treatment of Dengue virus, when administered to a host, such as a human, in need thereof. In certain embodiments, Compound 1 or Compound 2 can be provided in a pharmaceutical composition.

Further, it has been discovered that Compound 1 or Compound 2 are also active against Dengue serotypes 1, 3 and 4. With this new discovery, it is confirmed that Compound 1 and Compound 2 in the high dose regimens described herein possess potency against all serotypes of Dengue virus (Dengue virus serotype 1, 2, 3, and 4).

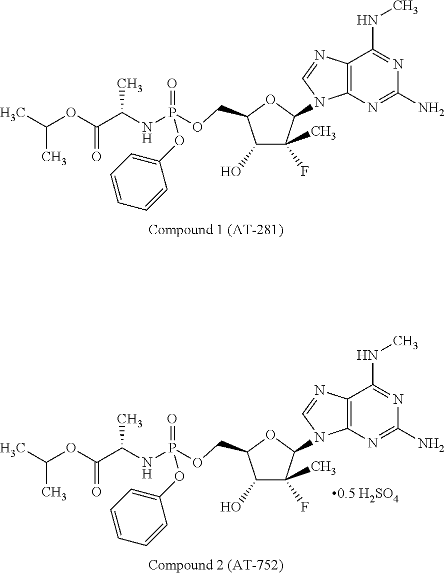 [SMILES: CNc1nc(N)nc2c1ncn2[C@@H]1O[C@H](COP(=O)(N[C@@H](C)C(=O)OC(C)C)Oc2ccccc2)[C@@H](O)[C@@]1(C)F ; CNc1nc(N)nc2c1ncn2[C@@H]1O[C@H](COP(=O)(N[C@@H](C)C(=O)OC(C)C)Oc2ccccc2)[C@@H](O)[C@@]1(C)F]

The term “Compound 1” as used in the specification can refer to either the free base or a pharmaceutically acceptable salt thereof. Compound 1 is isopropyl ((R)-(((2R,3R,4R,5R)-5-(2-amino-6-(methylamino)-9H-purin-9-yl)-4-fluoro-3-hydroxy-4-methyltetrahydrofuran-2-yl) methoxy)(phenoxy)phosphoryl)-L-alaninate.

It has also been discovered that Compound 1 (and its hemisulfate salt Compound 2) have an advantageous dual mechanism of action. The active metabolite is an inhibitor of the RNA dependent RNA polymerase (RdRp) domain of the viral protein NS5 as well as an inhibitor of the 2′-O-methyltransferase (MTase) domain of NS5. Both functions of the NS5 protein are required for replication. Inhibition of two key protein functions involved in RNA replication decreases the chance of a resistant mutant forming.

Compound 1 and 2 have an advantageous dual mechanism of action. It is an inhibitor of the RNA dependent RNA polymerase (RdRp) domain of the viral protein NS5 as well as an inhibitor of the 2′-O-methyltransferase (MTase) domain of NS5. Both functions of the NS5 protein are required for replication. Inhibition of two key protein functions involved in RNA replication decreases the chance of a resistant mutant from forming.

Compound 2 (AT-752) is the hemi-sulfate salt of Compound 1 (AT-281), a phosphoramidate protide which forms the L-alanyl metabolite Compound 3 (AT-551) as an intermediate prodrug before being converted to the 5′-monophosphate (MP) metabolite Compound 4 (AT-8001) of the nucleoside 2′-fluoro-2′-C-methylguanosine (Compound 6 (AT-273)). Compound 4 (AT-8001) is then phosphorylated to the active 5′-triphosphate (TP) metabolite Compound 5 (AT-9010). Compound 5 (AT-9010) has been shown to selectively inhibit the viral RNA-dependent RNA polymerase (RdRp) of HCV (Good et al., Preclinical evaluation of AT-527, a novel guanosine nucleotide prodrug with potent, pan-genotypic activity against hepatitis C virus. PLos One 15: e0227104), and here it is shown that it inhibits serotype 1, 2, 3, and 4 of Dengue. One mechanism is the inhibition of NS5, the RdRp of DENV-2.

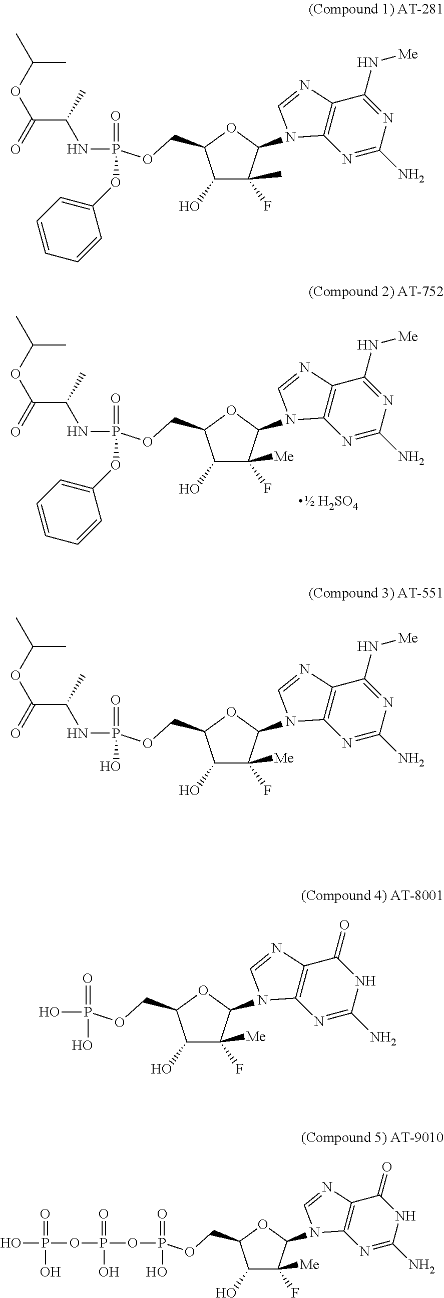

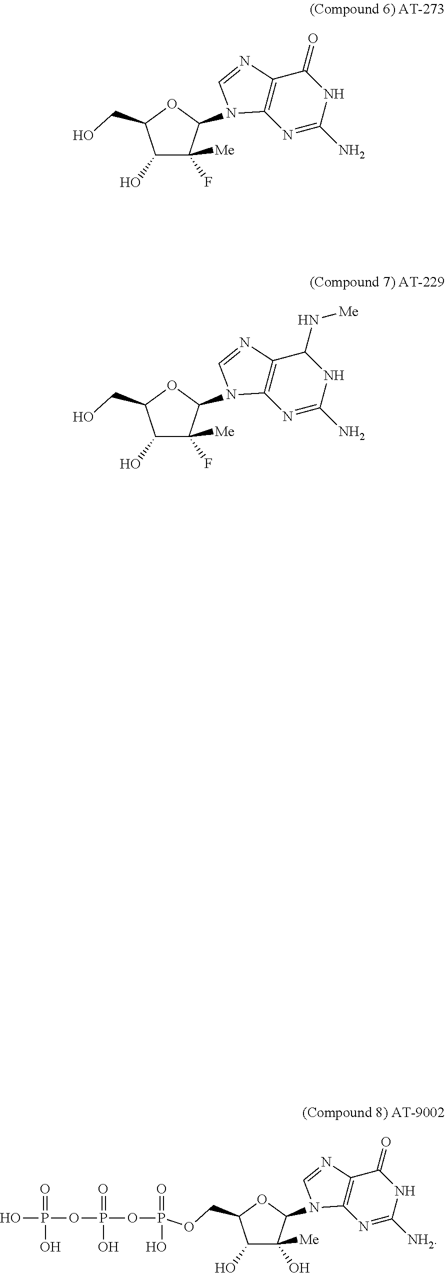 [SMILES: C[C@@]1(F)[C@H](O)[C@@H](CO)O[C@H]1n1cnc2c(=O)[nH]c(N)nc21 ; CNC1NC(N)=Nc2c1ncn2[C@@H]1O[C@H](CO)[C@@H](O)[C@@]1(C)F ; C[C@@]1(O)[C@H](O)[C@@H](COP(=O)(O)OP(=O)(O)OP(=O)(O)O)O[C@H]1n1cnc2c(=O)[nH]c(N)nc21]

In certain aspects, dosage regimens, methods, compositions, and processes of manufacture are provided for the treatment of a host infected with Dengue virus. For example, Compound 1 or 2 or Formula I or II can be administered in a dose of about 700-1000 mg, for example a dose of 750 mg two or three times a day, alone or in combination with another anti-RNA viral agent to treat the infected host in need thereof. In certain embodiments, it is useful to administer a combination of drugs that modulate the same or a different pathway or inhibit a different target in the virus. As Compound 1/2 is a polymerase inhibitor, it can be advantageous to administer Compound 1/2 to a host in combination with a protease inhibitor or an NS3-NS4B interaction inhibitor. Alternatively, Compound 1/2 can be administered in combination with a structurally different polymerase inhibitor.

Compound 1/2 can be administered orally, for example in pill or tablet form, and may be administered via another route which the attending physician considers appropriate, including via intravenous, transdermal, subcutaneous, topical, parenteral, or other suitable route.

### I. Definitions

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. The materials, methods, and examples are illustrative only and are not intended to be limiting.

Unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

A “patient” or “host” or “subject” is a human or non-human animal in need of treatment or prevention of a Dengue virus infection. Typically, the host is a human. A “patient” or “host” or “subject” also refers to, for example, a mammal, primate (e.g., human), cow, sheep, goat, horse, dog, cat, rabbit, rat, mice, bird and the like.

The term “prophylactic” or “preventative” when used refers to the administration of an active NS5 RNA dependent RNA polymerase (RdRp) inhibitory compound to prevent, reduce the likelihood of an occurrence or a reoccurrence of a Dengue virus infection such as Dengue 2 virus or Dengue 3 virus, or to minimize a new infection relative to infection that would occur without such treatment or to minimize transmission to another person. The present invention includes both treatment and prophylactic or preventative therapies. In some embodiments, the active NS5 RdRp inhibitory compound is administered to a host who has been exposed to and is thus at risk of contracting a Dengue virus infection such as Dengue 1 virus, Dengue 2 virus, Dengue 3 virus, or Dengue 4 virus. In another alternative embodiment, a method to prevent transmission is provided that includes administering an effective amount of one of the compounds described herein to humans for a sufficient length of time prior to exposure to crowds that can be infected, including during travel or public events or meetings, including for example, up to 3, 5, 7, 10, 12, 14 or more days prior to a communicable situation.

The terms “coadminister,” “coadministration,” or “in combination” are used to describe the administration of a NS5 RdRp interfering compound in combination with at least one other antiviral active agent. The timing of the coadministration is best determined by the medical specialist treating the patient. It is sometimes desired that the agents are administered at the same time. Alternatively, the drugs selected for combination therapy are administered at different times to the patient. Of course, when more than one viral or other infection or other condition is present, the present compounds may be combined with other agents to treat that other infection or condition as required.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the salts and the quaternary ammonium salts of the parent compound formed, for example, from inorganic or organic acids that are not unduly toxic. For example, acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n-COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

The compound can be delivered in any molar ratio of salt that delivers the desired result. For example, the compound can be provided with less than a molar equivalent of a counter ion, such as in the form of a hemi-sulfate salt. Alternatively, the compound can be provided with more than a molar equivalent of counter ion, such as in the form of a di-sulfate salt. Non-limiting examples of molar ratios of the compound to the counter ion include 1:0.25, 1:0.5, 1:1, and 1:2.

In certain embodiments, the term “about” means plus or minus 5% of the listed value. In certain embodiments, the term “about” means plus or minus 10% of the listed value.

### I. Advantageous Dosage Forms of Compound 2 for the Treatment of Dengue Virus

Compositions, methods, and dosage forms are provided for the treatment of a host infected with Dengue via administration of an effective amount of Compound 1 or Compound 2.

In an aspect of the invention, pharmaceutical compositions according to the present invention comprise an anti-Dengue virus high-dose of Compound 1 or 2 as described herein, optionally in combination with a pharmaceutically acceptable carrier, additive, or excipient, further optionally in combination or alternation with at least one other active compound. In one embodiment, the invention includes a solid dosage form of Compound 2 in a pharmaceutically acceptable carrier.

In certain embodiments Compound 1 or 2 is administered to a patient in need thereof in a pharmaceutical composition, for example Composition A or Composition B.

Composition A

| Ingredient | % w/w | mg/tablet |
|---|---|---|
| Compound 2 | 38.7% | 271.1 |
| Silicified Microcrystalline | 55.3% | 386.9 |
| Cellulose (Prosolv HD 90) | | |
| Croscarmellose Sodium (Ac-Di-Sol) | 1.0% | 35.0 |
| Magnesium Stearate | 1.00% | 7.0 |
| Total | 100% | 700 |

Composition B

| | |
|---|---|
| Ingredient | % w/w |

Intragranular

| | |
|---|---|
| Compound 2 | 46.3% |
| Silicified Microcrystalline Cellulose (Prosolv HD 90) | 40.8% |
| Colloidal Silicon Dioxide (Aerosil 200) | 4.2% |
| Croscarmellose Sodium (Ac-Di-Sol) | 1.0% |
| Sodium Stearyl Fumarate (PRUV) | 1.0% |

Extragranular

| | |
|---|---|
| Silicified Microcrystalline Cellulose (Prosolv HD 90) | 5.0% |
| Colloidal Silicon Dioxide (Aerosil 200) | 0.5% |
| Croscarmellose Sodium (Ac-Di-Sol) | 0.4% |
| Sodium Stearyl Fumarate (PRUV) | 0.8% |
| Total | 100.0% |

In an aspect of the invention, pharmaceutical compositions according to the present invention comprise an anti-Dengue high-dose effective amount of Compound 1 or 2 described herein, optionally in combination with a pharmaceutically acceptable carrier, additive, or excipient, further optionally in combination with at least one other antiviral agent, such as another anti-Dengue agent.

The invention includes pharmaceutical compositions that include an effective amount to treat a Dengue virus infection of Compound 1 or 2 of the present invention or prodrug, in a pharmaceutically acceptable carrier or excipient. In an alternative embodiment, the invention includes pharmaceutical compositions that include an effective amount to prevent a Dengue virus infection of Compound 1 or 2 of the present invention or prodrug, in a pharmaceutically acceptable carrier or excipient.

One of ordinary skill in the art will recognize that a therapeutically effective high dose amount will vary with the infection or condition to be treated, its severity, the treatment regimen to be employed, the pharmacokinetic of the agent used, as well as the patient or subject (animal or human) to be treated, and such therapeutic amount can be determined by the attending physician or specialist.

Compound 1 or 2 according to the present invention can be formulated in a mixture with a pharmaceutically acceptable carrier. In general, it is typical to administer the pharmaceutical composition in orally-administrable form, and in particular, a solid dosage form such as a pill or tablet. Certain formulations may be administered via a parenteral, intravenous, intramuscular, topical, transdermal, buccal, subcutaneous, suppository, or other route, including intranasal spray. Intravenous and intramuscular formulations are often administered in sterile saline. One of ordinary skill in the art may modify the formulations to render them more soluble in water or another vehicle, for example, this can be easily accomplished by minor modifications (salt formulation, esterification, etc.) that are well within the ordinary skill in the art. It is also well within the routineers' skill to modify the route of administration and dosage regimen of Compound 2 in order to manage the pharmacokinetic of the present compounds for maximum beneficial effect in patients, as described in more detail herein.

Compound 1 or 2 may be provided, for example, for oral or parenteral delivery. In certain embodiments, Compound 1 or 2 is provided in a solid, gel, or liquid dosage form. In certain embodiments, Compound 1 or 2 is provided in a liquid dosage form for parenteral administration. In certain embodiments, Compound 1 or 2 is provided in a liquid dosage form for intravenous administration. In certain embodiments, Compound 1 or 2 is provided in a solid dosage form for oral administration. In certain embodiments, Compound 1 or 2 may be provided as a softshell capsule or as a tablet for oral administration.

The high dose amount of Compound 1 or 2 included within the therapeutically active formulation according to the present invention is described in detail herein and is an effective amount to achieve the desired outcome according to the present invention, for example, for treating the DENV infection, reducing the likelihood of a DENV infection or the inhibition, reduction, and/or abolition of DENV or its secondary effects, including disease states, conditions, and/or complications which occur secondary to DENV.

Doses described herein refer to the amount of active pharmaceutical ingredient in the dose with the weight added by pharmaceutically acceptable salts, unless otherwise indicated. For example, a 750 mg dose of Compound 2 comprises about 692 mg of Compound 1 (which falls within “about 700 mg”) when the molecular weight of the hemisulfate salt is factored into the measurement.

In general, a therapeutically effective amount of Compound 2 in a pharmaceutical dosage form may range from about 600 mg to about 1,000 mg, about 700 mg to about 1,000 mg, or about 700 mg to about 800 or 850 mg when measured as the free base. In certain embodiments, Compound 1 is administered in the same range as the free base.

In certain embodiments, Compound 2 is administered in dose amounts of at least about 650 mg, 700 mg, 750 mg, 800 mg, 850 mg, 900 mg, 950 mg, 1,000 mg, 1,150 mg, 1,200 mg, 1,250 mg, 1,300 mg, 1,350 mg, 1,400 mg, 1,450 mg, or 1,500 mg when measured as the free base two or three times a day. In some embodiments, the dose is provided in several dosage forms to reduce the size of the solid dosage form such as a pill, tablet or capsule.

In certain embodiments, about 700 mg to about 800 or 850 mg of Compound 1 or 2 is administered once, twice or three times per day (QD). In certain embodiments, about 700 mg to about 800 mg of Compound 1 or 2 is administered once, twice or three times per day (QD). In certain embodiments, about 800 mg to about 900 mg of Compound 2 is administered once per day (QD). In certain embodiments, about 900 mg to about 1,000 mg of Compound 2 is administered once per day (QD).

In certain embodiments, about 700 mg to about 800 mg of Compound 2 is administered twice per day (BID). In certain embodiments, about 700 mg to about 900 mg of Compound 2 is administered twice per day (BID). In certain embodiments, about 800 mg to about 900 mg of Compound 2 is administered twice per day (BID). In certain embodiments, about 900 mg to about 1,000 mg of Compound 2 is administered twice per day (BID).

In certain embodiments, about 600 mg to about 700 mg of Compound 2 is administered three times per day (TID). In certain embodiments, about 700 mg to about 800 mg of Compound 2 is administered three times per day (TID). In certain embodiments, about 800 mg to about 900 mg of Compound 2 is administered three times per day (TID). In certain embodiments, about 900 mg to about 1,000 mg of Compound 2 is administered three times per day (TID).

In certain embodiments, about 700 mg to about 900 mg of Compound 2 is administered four times per day (QID). In certain embodiments, about 600 mg to about 700 or 750 mg of Compound 2 is administered four times per day (QID). In certain embodiments, about 700 mg to about 800 mg of Compound 2 is administered four times per day (QID). In certain embodiments, about 800 mg to about 900 mg of Compound 2 is administered three four per day (QID). In certain embodiments, about 900 mg to about 1,000 mg of Compound 2 is administered four times per day (QID).

For purposes of the present invention, a prophylactically or preventive effective amount of the compositions according to the present invention falls within the same high-dose concentration range as set forth above for therapeutically effective amount and is usually the same as a therapeutically effective amount.

In certain embodiments, the Dengue virus is serotype 1, 3, or 4. In certain embodiments, the Dengue virus is serotype 1, 3, or 4 and about 700 mg to about 1000 mg of Compound 1 or 2 is administered once per day (QD). In certain embodiments, the Dengue virus is serotype 1, 3, or 4 and about 550 mg to about 750 mg of Compound 2 is administered once per day (QD). In certain embodiments, the Dengue virus is serotype 1, 3, or 4 and about 600 mg of Compound 1 or 2 is administered once per day (QD).

In certain embodiments, the Dengue virus is serotype 1, 3, or 4 and about 500 mg to about 800 mg of Compound 1 or 2 is administered twice per day (BID). In certain embodiments, the Dengue virus is serotype 1, 3, or 4 and about 550 mg to about 750 mg of Compound 1 or 2 is administered twice per day (BID). In certain embodiments, the Dengue virus is serotype 1, 3, or 4 and about 600 mg of Compound 1 or 2 is administered twice per day (BID).

In certain embodiments, the Dengue virus is serotype 1, 3, or 4 and about 500 mg to about 800 mg of Compound 1 or 2 is administered three times per day (TID). In certain embodiments, the Dengue virus is serotype 1, 3, or 4 and about 550 mg to about 750 mg of Compound 1 or 2 is administered three times per day (TID). In certain embodiments, the Dengue virus is serotype 1, 3, or 4 and about 600 mg of Compound 1 or 2 is administered three times per day (TID).

Administration of Compound 1 or 2 may range from continuous (intravenous drip) to several oral or intranasal administrations per day (e.g., two, three, four, or five times per day) or transdermal administration and may include oral, topical, parenteral, intramuscular, intravenous, sub-cutaneous, transdermal (which may include a penetration enhancement agent), buccal, and suppository administration, among other routes of administration. Enteric coated oral tablets may also be used to enhance bioavailability of the compounds for an oral route of administration. The most effective dosage form will depend upon the bioavailability/pharmacokinetic of the particular agent chosen as well as the severity of disease in the patient. Oral dosage forms are particularly preferred, because of ease of administration and prospective favorable patient compliance.

To prepare the pharmaceutical compositions according to the present invention, a therapeutically effective amount of Compound 1 or 2 according to the present invention can be intimately admixed with a pharmaceutically acceptable carrier according to conventional pharmaceutical compounding techniques to produce a dose. A carrier may take a wide variety of forms depending on the form of preparation desired for administration, e.g., oral or parenteral. In preparing pharmaceutical compositions in oral dosage form, any of the usual pharmaceutical excipients and components may be used. Thus, for liquid oral preparations such as suspensions, elixirs, and solutions, suitable carriers and additives including water, glycols, oils, alcohols, flavoring agents, preservatives, coloring agents, and the like may be used. For solid oral preparations such as powders, tablets, capsules, and for solid preparations such as suppositories, suitable carriers and additives including starches, sugar carriers, such as dextrose, manifold, lactose, and related carriers, diluents, granulating agents, lubricants, binders, disintegrating agents, and the like may be used. If desired, the tablets or capsules may be enteric-coated or sustained release by standard techniques. The use of these dosage forms may significantly enhance the bioavailability of the compounds in the patient.

For parenteral formulations, the carrier will usually comprise sterile water or aqueous sodium chloride solution, though other ingredients, including those which aid dispersion, also may be included. Of course, where sterile water is to be used and maintained as sterile, the compositions and carriers must also be sterilized. Injectable suspensions may also be prepared, in which case appropriate liquid carriers, suspending agents, and the like may be employed.

Liposomal suspensions (including liposomes targeted to viral antigens) may also be prepared by conventional methods to produce pharmaceutically acceptable carriers. This may be appropriate for the delivery of free nucleosides, acyl/alkyl nucleosides or phosphate ester pro-drug forms of the nucleoside compounds according to the present invention.

In typical embodiments according to the present invention, Compound 1 or 2 and the compositions described are used to treat, prevent or delay a Dengue infection or a secondary disease state, condition or complication of Dengue.

### Isotopic Substitution

The present invention includes compounds and the use of Compound 1 or Compound 2 with desired isotopic substitutions of atoms at amounts above the natural abundance of the isotope, i.e., enriched. Isotopes are atoms having the same atomic number but different mass numbers, i.e., the same number of protons but a different number of neutrons. By way of general example and without limitation, isotopes of hydrogen, for example, deuterium (²H) and tritium (³H) may be used anywhere in described structures. Alternatively, or in addition, isotopes of carbon, e.g., ¹³C and ¹⁴C, may be used. A preferred isotopic substitution is deuterium for hydrogen at one or more locations on the molecule to improve the performance of the drug. The deuterium can be bound in a location of bond breakage during metabolism (an α-deuterium kinetic isotope effect) or next to or near the site of bond breakage (a β-deuterium kinetic isotope effect). Achillion Pharmaceuticals, Inc. (WO/2014/169278 and WO/2014/169280) describes deuteration of nucleotides to improve their pharmacokinetics or pharmacodynamics, including at the 5-position of the molecule.

Substitution with isotopes such as deuterium can afford certain therapeutic advantages resulting from greater metabolic stability, such as, for example, increased in vivo half-life or reduced dosage requirements. Substitution of deuterium for hydrogen at a site of metabolic break-down can reduce the rate of or eliminate the metabolism at that bond. At any position of the compound that a hydrogen atom may be present, the hydrogen atom can be any isotope of hydrogen, including protium (¹H), deuterium (H) and tritium (3H). Thus, reference herein to a compound encompasses all potential isotopic forms unless the context clearly dictates otherwise.

The term “isotopically-labeled” analog refers to an analog that is a “deuterated analog”, a “¹³C-labeled analog,” or a “deuterated/¹³C-labeled analog.” The term “deuterated analog” means a compound described herein, whereby an H-isotope, i.e., hydrogen/protium (¹H), is substituted by a H-isotope, i.e., deuterium (H). Deuterium substitution can be partial or complete. Partial deuterium substitution means that at least one hydrogen is substituted by at least one deuterium. In certain embodiments, the isotope is 90, 95 or 99% or more enriched in an isotope at any location of interest. In some embodiments it is deuterium that is 90, 95 or 99% enriched at a desired location. Unless indicated to the contrary, the deuteration is at least 80% at the selected location. Deuteration of the nucleoside can occur at any replaceable hydrogen that provides the desired results.

In certain aspects of the present invention, a compound of Formula I:

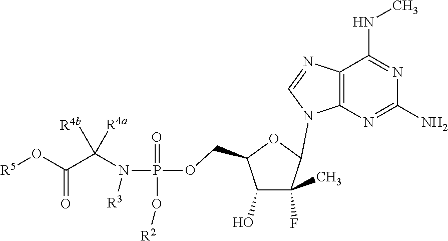

or a pharmaceutically acceptable salt thereof optionally in a pharmaceutically acceptable carrier, is administered in a high dose effective amount as described herein to treat a Dengue virus infection in a host in need thereof, typically a human;
wherein:
R² is C₁₋₆alkyl (including methyl, ethyl, propyl, and isopropyl), C₃₋₇cycloalkyl, aryl, aryl(C₁-C₄alkyl)-, heteroaryl, heteroaryl(C₁-C₄alkyl)-, or heteroalkyl, each of which is optionally substituted with 1, 2, 3, or 4 substituents independently selected at each occurrence from R²²;
R³ is hydrogen or C₁₋₆alkyl optionally substituted with 1, 2, 3, or 4, substituents independently selected at each occurrence from R³³;
R^{4a} and R^{4b} are independently selected from hydrogen, C₁₋₆alkyl, aryl(C₁₋₆alkyl)-, C₃₋₇cycloalkyl, C₃₋₇cycloalkyl(C₁₋₆alkyl)-, heteroaryl(C₁₋₆alkyl)-, and aryl, each of which is optionally substituted with 1, 2, 3, or 4 substituents independently selected at each occurrence from R⁴⁴;
R⁵ is hydrogen, C₁₋₆alkyl (including methyl, ethyl, propyl, and isopropyl), C₁₋₆haloalkyl, or C₃₋₇cycloalkyl and in an alternative embodiment, R⁵ is aryl(C₁-C₄alkyl)-, aryl, heteroaryl, or heteroalkyl, optionally substituted with 1, 2, 3, or 4 substituents independently selected at each occurrence from R⁵⁵.

R²², R³³, R⁴⁴, and R⁵⁵ are independently selected at each occurrence from hydrogen, C₁. C₆alkyl, C₃-C₇cycloalkyl, alkenyl, alkynyl, F, Cl, Br, I, heterocycle, heterocycloalkyl, haloalkoxy, haloalkyl, C(O)R⁶⁶, C(O)OR⁶⁶, C(O)N(R⁶⁶)₂, and cyano;
R⁶⁶ is independently selected at each occurrence from hydrogen, C₁-C₆alkyl, aryl(C₁-C₆alkyl)-, C₃-C₇cycloalkyl, C₃-C₇cycloalkyl(C₁-C₆alkyl)-, heteroaryl(C₁-C₆alkyl)-, and aryl.

All R groups are intended to be interpreted in a manner that does not include redundancy i.e., as known in the art, alkyl would not be substituted with alkyl; however, for example, alkoxy substituted with alkoxy is not redundant. Aryl substituted with aryl falls within the definition of aryl in a limited scope. R groups are not optionally substituted unless specifically indicated in context.

Non-limiting examples of C₁-C₆alkyl include methyl, ethyl, propyl, isopropyl, butyl, t-butyl, sec-butyl, isobutyl, —CH₂C(CH₃)₃, —CH(CH₂CH₃)₂, and —CH₂CH(CH₂CH₃)₂. Non-limiting examples of C₃-C₆cycloalkyl include cyclopropyl, CH₂-cyclopropyl, cyclobutyl, and CH₂-cyclobutyl.

A non-limiting example of aryl(C₁-C₄alkyl)- is benzyl. Non-limiting examples of aryl are phenyl and naphthyl.

In certain embodiments, Formula I is:

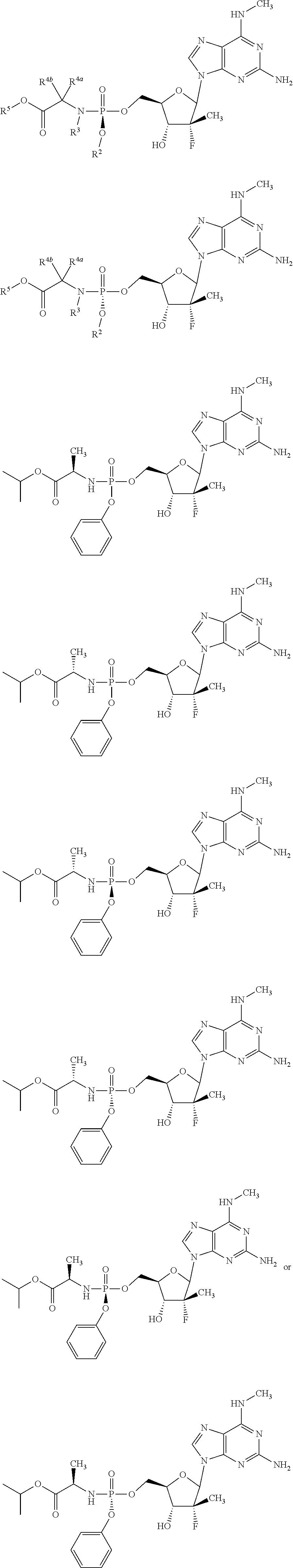

In certain aspects of the present invention, a compound of Formula II:

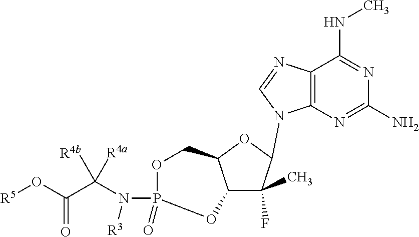

or a pharmaceutically acceptable salt thereof optionally in a pharmaceutically acceptable carrier, is administered in an effective amount to treat a Dengue virus infection in a host in need thereof, typically a human; and all variables are as defined herein.

In certain aspects, the compound is of the Formula IIA:

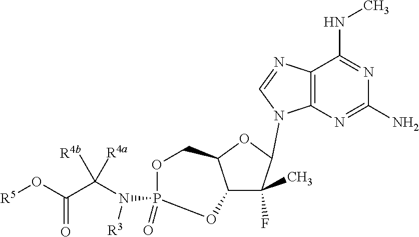

or a pharmaceutically acceptable salt thereof, wherein all variables are as defined herein.

In certain embodiments, the compound of Formula IIA is selected from:

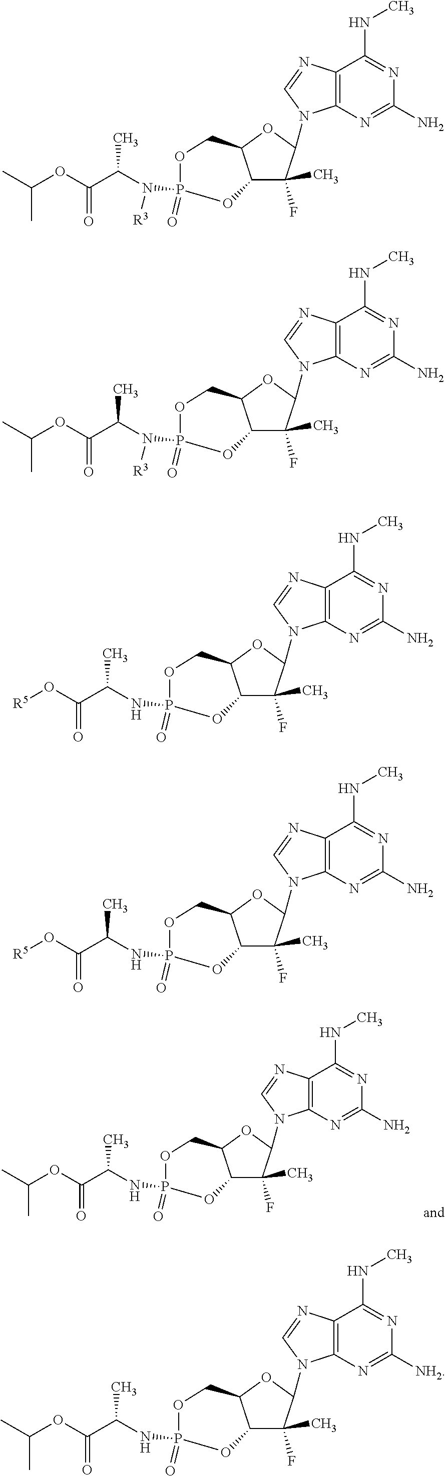

In certain aspects, the compound is of the Formula IIB:

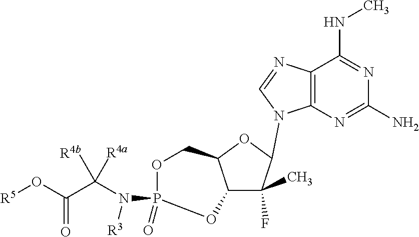

or a pharmaceutically acceptable salt thereof, wherein all variables are as defined herein.

In certain embodiments, the compound of Formula IIB is selected from:

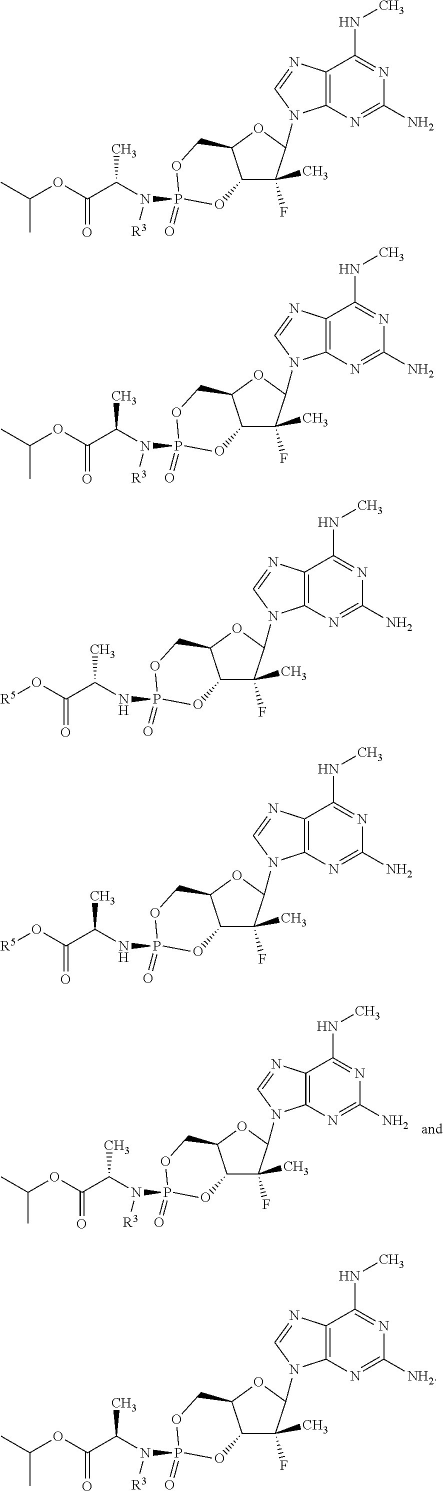

In certain embodiments, a compound of Formula II, Formula IIA, or Formula IIB is used in the treatment of an infection of DENV1, DENV2, DENV3, or DENV4.

In certain embodiments, R^{4a} is selected to be the side chain of a naturally occurring amino acid, for example:

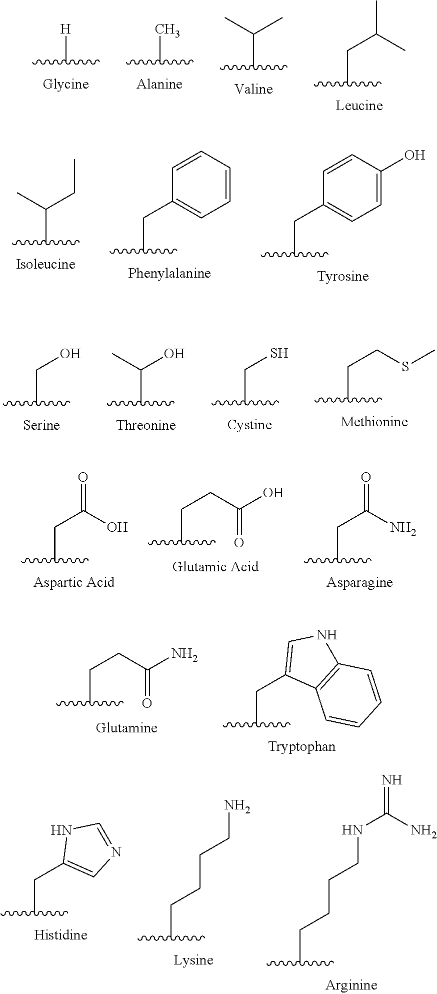

In certain alternative embodiments, variable R^{4a} of Formula II, IIA, or IIB is C₇-C₁₅alkyl, variable R^{4b} is hydrogen, and all other variables are as defined herein. In alternative embodiments, the compound is selected from

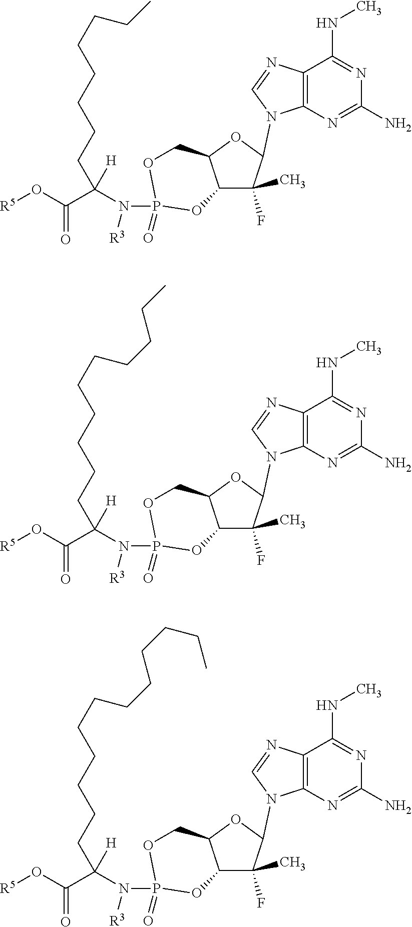

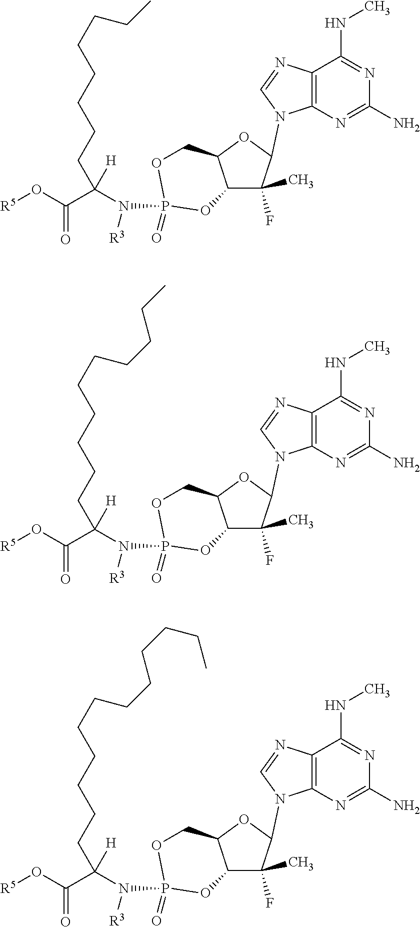

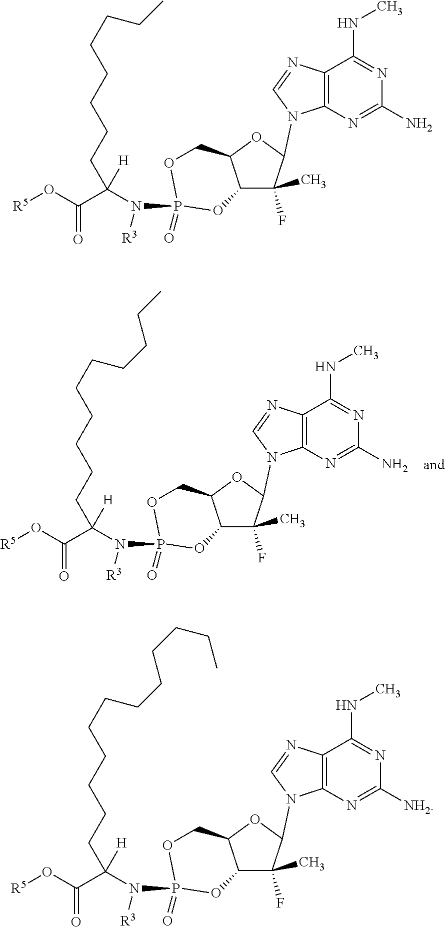

### II. Methods of Treatment or Prophylaxis

Treatment, as used herein, refers to the administration of Compound 1 or Compound 2 to a host, for example a human that is or may become infected with Dengue virus.

The term “prophylactic” or preventative, when used, refers to the administration of Compound 1 or 2 to prevent or reduce the likelihood of an occurrence of the viral disorder. The present invention includes both treatment and prophylactic or preventative therapies. In one embodiment, Compound 1 or 2 is administered to a host who has been exposed to and thus is at risk of infection by a Dengue virus infection.

The invention is directed to a method of treatment or prophylaxis of a Dengue virus, including drug resistant and multidrug resistant forms of DENV and related disease states, conditions, or complications. The method comprises administering to a host in need thereof, typically a human, with an effective amount of Compound 1 or 2 as described herein, optionally in combination with at least one additional bioactive agent, for example, an additional anti-DENV agent, further in combination with a pharmaceutically acceptable carrier additive and/or excipient.

In yet another aspect, the present invention is a method for prevention or prophylaxis of a Dengue infection or a disease state or related or follow-on disease state, condition or complication of a Dengue infection.
(1) In certain embodiments a dosage regimen for use in the treatment of human host in need thereof infected with a Dengue virus or for the prevention of Dengue virus comprising administering about 700, 725, 750, 775, 800, 825, 850, 875, 900, 925, 950, 975, 1000, 1025, 1050, 1075, 1100, 1125, 1150, 1175, 1200, 1225, or 1250 mg of Compound 1 or Compound 2 two, three, or four times a day is provided.
(2) In certain embodiments a dosage regimen for use in the treatment of human host in need thereof infected with a Dengue virus or for the prevention of Dengue virus comprising administering about 700, 725, 750, 775, 800, 825, 850, 875, 900, 925, 950, 975, 1000, 1025, 1050, 1075, 1100, 1125, 1150, 1175, 1200, 1225, or 1250 mg of Compound 1 or 2 two, three, or four times a day is provided.
(3) In certain embodiments a dosage regimen for use in the treatment of human host in need thereof infected with a Dengue virus or for the prevention of Dengue virus comprising administering about 700, 725, 750, 775, 800, 825, 850, 875, 900, 925, 950, 975, 1000, 1025, 1050, 1075, 1100, 1125, 1150, 1175, 1200, 1225, or 1250 mg of Compound 1 or 2 two three, or four times a day is provided.
(4) Any one of embodiments (1)-(3) wherein Compound 1 or Compound 2 is administered two times a day.
(5) Any one of embodiments (1)-(3) wherein Compound 1 or Compound 2 is administered three times a day.
(6) Any one of embodiments (1)-(3) wherein Compound 1 or Compound 2 is administered four times a day.
(7) Any one of embodiments (1)-(6) wherein about 600 mg of Compound 1 or Compound 2 is administered in each dose to the patient in need thereof.
(8) Any one of embodiments (1)-(6) wherein about 650 mg of Compound 1 or Compound 2 is administered in each dose to the patient in need thereof.
(9) Any one of embodiments (1)-(6) wherein about 700 mg of Compound 1 or Compound 2 is administered in each dose to the patient in need thereof.
(10) Any one of embodiments (1)-(6) wherein about 750 mg of Compound 1 or Compound 2 is administered in each dose to the patient in need thereof.
(11) Any one of embodiments (1)-(6) wherein about 800 mg of Compound 1 or Compound 2 is administered in each dose to the patient in need thereof.
(12) Any one of embodiments (1)-(6) wherein about 850 mg of Compound 2 or Compound 1 is administered in each dose to the patient in need thereof.
(13) Any one of embodiments (1)-(6) wherein about 900 mg of Compound 1 or Compound 2 is administered in each dose to the patient in need thereof.
(14) Any one of embodiments (1)-(6) wherein about 950 mg of Compound 1 or Compound 2 is administered in each dose to the patient in need thereof.
(15) Any one of embodiments (1)-(6) wherein about 1000 mg of Compound 1 or Compound 2 is administered in each dose to the patient in need thereof.
(16) Any one of embodiments (1)-(15) wherein Composition A is administered.
(17) Any one of embodiments (1)-(15) wherein Composition B is administered.
(18) Any one of embodiments (1)-(17) wherein Compound 1 or Compound 2 is administered for three consecutive days.
(19) Any one of embodiments (1)-(17) wherein Compound 1 or Compound 2 is administered for four consecutive days.
(20) Any one of embodiments (1)-(17) wherein Compound 1 or Compound 2 is administered for five consecutive days.
(21) Any one of embodiments (1)-(17) wherein Compound 1 or Compound 2 is administered for six consecutive days.
(22) Any one of embodiments (1)-(17) wherein Compound 1 or Compound 2 is administered for seven consecutive days.
(23) Any one of embodiments (1)-(17) wherein Compound 1 or Compound 2 is administered for eight consecutive days.
(24) Any one of embodiments (1)-(17) wherein Compound 1 or Compound 2 is administered for nine consecutive days.
(25) Any one of embodiments (1)-(17) wherein Compound 1 or Compound 2 is administered for ten consecutive days.
(26) Any one of embodiments (1)-(17) wherein Compound 1 or Compound 2 is administered for eleven consecutive days.
(27) Any one of embodiments (1)-(17) wherein Compound 1 or Compound 2 is administered for twelve consecutive days.
(28) Any one of embodiments (1)-(17) wherein Compound 1 or Compound 2 is administered for thirteen consecutive days.
(29) Any one of embodiments (1)-(17) wherein Compound 1 or Compound 2 is administered for fourteen consecutive days.
(30) Any one of embodiments (1)-(29) wherein the method is used prophylactically on a healthy patient to prevent infection.
(31) Any one of embodiments (1)-(29) wherein the method is used to treat a patient with Dengue virus.
(32) Any one of embodiments (1)-(29) wherein the human has Dengue serotype 1.
(33) Any one of embodiments (1)-(29) wherein the human has Dengue serotype 2.
(34) Any one of embodiments (1)-(29) wherein the human has Dengue serotype 3.
(35) Any one of embodiments (1)-(29) wherein the human has Dengue serotype 4.

In an alternative embodiment, Compound 2 is provided as the hemisulfate salt of a phosphoramidate of Compound 1 other than the specific phosphoramidate described in the compound illustration such as in Formula I or II. A wide range of phosphoramidates are known to those skilled in the art that include various esters and phospho-esters, any combination of which can be used to provide an active compound as described herein in the form of a hemisulfate salt.

### VI. Combination and Alternation Therapy

It is well recognized that drug-resistant variants of viruses can emerge after prolonged treatment with an antiviral agent. Drug resistance sometimes occurs by mutation of a gene that encodes for an enzyme used in viral replication. The efficacy of a drug against DENV infection, can be prolonged, augmented, or restored by administering the compound in combination or alternation with another, and perhaps even two or three other, antiviral compounds that induce a different mutation or act through a different pathway, from that of the principle drug.

Alternatively, the pharmacokinetic, bio distribution, half-life, or other parameter of the drug can be altered by such combination therapy (which may include alternation therapy if considered concerted). Since the disclosed Compound 2 and Compound 1 is an NS5 polymerase inhibitor, it may be useful to administer the compound to a host in combination with, for example a
(1) Protease inhibitor, such as an NS2B-NS3 protease inhibitor;
(2) NS3 inhibitor;
(3) NS3-NS4B interaction inhibitor;
(4) NS4B inhibitor;
(5) NS3-NS5 interactions inhibitor;
(6) Another NS5 polymerase inhibitor;
(7) Interferon alfa-2a, which may be pegylated or otherwise modified, and/or ribavirin;
(8) Non-substrate-based inhibitor;
(9) Helicase inhibitor;
(10) Antisense oligodeoxynucleotide (S-ODN);
(11) Aptamer;
(12) Nuclease-resistant ribozyme;
(13) iRNA, including microRNA and SiRNA;
(14) Antibody, partial antibody or domain antibody to the virus, or
(15) Viral antigen or partial antigen that induces a host antibody response.
   Non limiting examples of Dengue DAA agents that can be administered in combination with high-dose Compound 1 or 2 or Formula I or II of the invention, alone or with multiple drugs from these lists, are
(i) protease inhibitor such as telaprevir (Incivek®), boceprevir (Victrelis™), simeprevir (Olysio™), paritaprevir (ABT-450), glecaprevir (ABT-493), ritonavir (Norvir), ACH-2684, AZD-7295, BMS-791325, danoprevir, Filibuvir, GS-9256, GS-9451, MK-5172, Setrobuvir, Sovaprevir, Tegobuvir, VX-135, VX-222, and, ALS-220;
(ii) NS3 inhibitor such as suramin, dabrafenib, idelalisib, nintedanib, ivermectin, or BP13944
(iii) NS3-NS4B interaction inhibitors such as JNJ-1802 and JNJ-A07;
(iv) NS4B inhibitor such as NITD-688 or lycorine;
(v) NS5 inhibitors such as balapiravir;
(vi) HCV NS5B inhibitors such as AZD-7295, Clemizole, dasabuvir (Exviera), ITX-5061, PPI-461, PPI-688, sofosbuvir (Sovaldi®), MK-3682, ABT-333, and MBX-700 and mericitabine;
(vii) Antiviral combination drugs such as Harvoni (ledipasvir/sofosbuvir); Viekira Pak (ombitasvir/paritaprevir/ritonavir/dasabuvir); Viekirax (ombitasvir/paritaprevir/ritonavir); G/P (paritaprevir and glecaprevir); Technivie (ombitasvir/paritaprevir/ritonavir) and Epclusa (sofosbuvir/velpatasvir) and Zepatier (elbasvir and grazoprevir).

### EXAMPLES

### Example 1. Cell Lines, Viruses and Test Compounds

The cell lines, viruses and compounds described herein were used to generate the data presented in Example 2, 3, 4, 5, 6, 7, and 8. Huh-7 (human liver carcinoma; AcceGen Biotechnology, Fairfield, NJ) cells were maintained in Dulbecco's Modified Eagle medium (DMEM) supplemented with 10% fetal bovine serum (FBS) and 100 μg/mL penicillin and 100 μg/mL streptomycin (Lonza, Walkersville, MD). Vero 76 cells (American Type Culture Collection (ATCC), Manassas, VA) used for the virus yield reduction assay were maintained similarly. BHK-21 (baby hamster kidney; ATCC, Manassas, VA) cells were maintained in Minimum Essential Medium with Earle's salts (EMEM) containing 1 mM sodium pyruvate and 25 μg/mL kanamycin, supplemented with 10% FBS. All cell cultures were maintained at 37° C. in an atmosphere of 5% CO₂ and ≥95% humidity. Infections were performed in EMEM supplemented with 5% FBS and 50 μg/mL gentamicin.

The Dengue viruses (DENV-2 NGC and DENV-3 H87) were obtained from ATCC (Manassas, VA), and DENV-2 used in the human PBMC and BHK-21 assays was a clinical isolate. Japanese encephalitis (JEV SA-14), West Nile (WNO2 Kern 515), Yellow fever (YFV 17D) and Zika (ZIKV MR766) viruses were obtained from the University of Texas Medical Branch (Galveston, TX) while the Powassan (POWV Spooner and LB strains) and Usutu (USUV TC-508) viruses were from the World Reference Center for Emerging Viruses and Arboviruses at the University of Texas Medical Branch. For studies using fresh human PBMCs, cells from a single male donor (Lot LS-88-45477C) were obtained from BioIVT (Westbury, NY) or were isolated from blood collected from healthy donors using Ficoll-Paque (Pham et al., 2008. Hepatitis C virus replicates in the same immune cell subsets in chronic hepatitis C and occult infection. Gastroenterology. 134:812-22, Pham et al., 2004. Hepatitis C virus persistence after spontaneous or treatment-induced resolution of hepatitis C. J Virol 78:5867-74). The DENV-2 studies in PBMCs and BHK21 cells were completed at University degli Studi di Cagliari (Monserrato, Italy). The other virus studies for efficacy and specificity were conducted at Utah State University (Logan, UT) and ImQuest BioSciences (Frederick, MD). Protocols at the latter laboratory for the different RNA and DNA viruses—HCV, HIV-1, Influenza A & B, RSV, Rhinovirus, Herpes simplex-1 and Adenovirus—have been previously described (Good et al., 2020. Preclinical evaluation of AT-527, a novel guanosine nucleotide prodrug with potent, pan-genotypic activity against hepatitis C virus. PLos One 15:30227104). Compound 2 (AT-752) and its free base Compound 1 (AT-281) were synthesized by a stereospecific process and, along with its plasma metabolite Compound 6 (AT-273), were prepared for Atea Pharmaceuticals by Topharman Shanghai Co., Ltd., Shanghai, China. (Compound 5 (AT-9010) and the TP internal standards used to quantify Compound 5 (AT-9010) were synthesized by NuBlocks (Oceanside, CA). Stock solutions were prepared in DMSO and stored at −20° C. Frozen plasma, human and from SD rat (BioreclamationlVT, Westbury, NY) and from Cynomolgus monkey (Suzhou Xishan, China) was purchased to test the stability of Compound 1 (AT-281) at 37° C.

### Example 2. Compound 1 Inhibits Dengue Virus (DENV) and Other Flaviviruses

The antiviral activity of Compound 1 (AT-281) was evaluated against flaviviruses DENV-2 NGC, DENV-3, JEV, POWV, USUV, WNV, YFV and ZIKV using a neutral red dye uptake assay (described in Example 3) to determine inhibition of virus-induced and compound-induced cytopathic effect (CPE) and using a virus yield reduction (VYR) assay (described in Example 4) as a second, independent determination of the inhibition of viral replication. Human hepatocarcinoma (Huh-7), baby hamster kidney (BHK-21) or human peripheral blood mononuclear cells (PBMCs) were acutely infected with the different viruses and exposed to serial dilutions of the drug (described in Example 5). The activity of Compound 1 (AT-281) was measured in infected cells using the neutral red assay and/or the virus yield reduction (VYR) assay, to determine the effective concentration required to achieve 50% inhibition (EC₅₀) of the virus-induced cytopathic effect (CPE), the concentration to reduce virus yield by 1 log₁₀ (EC₉₀) and the cytotoxic concentration of the drug to cause death to 50% of viable cells incubated without virus (CC₅₀) (see Table 1). After a 3- to 6-day incubation, the effective concentration of Compound 1 (AT-281), a 2′-fluoro-2′-C-methyl guanosine nucleotide prodrug, required to achieve 50% inhibition (EC₅₀) of the virus-induced cytopathic effect (CPE) ranged from 0.19 to 1.41 μM (see tables on next page).

TABLE 1A
Antiviral activity of Compound 1 (AT-281)
in cells infected with various flaviviruses

| | | | EC₅₀^{a} | EC₉₀^{b} | CC₅₀^{c} |
|---|---|---|---|---|---|
| Virus | Strain | Host Cell | (μM) | (μM) | (μM) |
| Dengue-2 (DENV-2) | New Guinea C | Huh-7 | 0.48 | 0.64 | >170 |
| | clinical isolate | Human PBMC | 0.80 | nd | >85 |
| | clinical isolate | BHK-21 | 1.1 | nd | >170 |
| Dengue-3 (DENV-3) | H87 | Huh-7 | 0.77 | nd | >170 |
| Japanese encephalitis | SA-14 | Huh-7 | 0.21 | nd | >170 |
| West Nile (WNV) | Kern 515, WNo2 | Huh-7 | 1.41 | 0.43 | >170 |
| Yellow Fever (YFV) | YFV 17D | Huh-7 | 0.31 | 0.26 | >170 |
| Zika | MR766 | Huh-7 | 0.64 | nd | >170 |
| Powassan | Spooner | BHK-21 | 0.29 | 0.74 | >170 |
| | LB | BHK-21 | 0.64 | 2.6 | >170 |
| Usutu | TC-508 | Huh-7 | 0.19 | 0.72 | >170 |

^{a}neutral red assay
^{b}VYR assay
^{c}highest concentration tested
nd, not determined

TABLE 1B
Antiviral activity of Compound 1 (AT-281) in cells
infected with various serotypes of Dengue

| Serotype | Strain | Host Cell | Read-out^{a} | EC₅₀ (μM) |
|---|---|---|---|---|
| DENV-1 | WP | Huh-7 | Luc | 0.57 |
| | WP74 | Huh-7 | FC | 0.15 |
| DENV-2 | NGC | Huh-7 | NR | 0.48 |
| | | Huh-7 | VYR | 0.64^{a} |
| | | Huh-7 | Luc | 0.30 |
| | | BHK-21 | CPE | 3.82 |
| | Clinical isolate | PBMC | CPE | 0.80 |
| | | BHK-21 | CPE | 1.05 |
| | | BHK-21 | VYR | 6.5^{a} |
| DENV-3 | H87 | Huh-7 | NR | 0.77 |
| | VN32 | Huh-7 | Luc | 0.75 |
| | CH4348 | Huh-7 | FC | 1.33 |
| DENV-4 | MY01 | Huh-7 | Luc | 0.57 |
| | TVP-360 | Huh-7 | FC | 1.19 |

^{a}neutral red assay

While the growth kinetics of different flaviviruses can differ in cell culture, Compound 1 (AT-281) showed similar potent effect in reducing viral titer against all 7 flaviviruses including 11 different strains tested in vitro (Table 1A) the antiviral activity was also maintained across Dengue serotype 1, 2, 3, and 4 across all tested strains (Table 1). The inhibition curves for DENV, WNV and YFV (FIG. 2) demonstrate this consistency. The concentration of Compound 1 (AT-281) required to kill 50% of cells exposed to drug only (CC₅₀) exceeded the highest concentration tested (85 to 170 μM), and the antiviral selectivity index (SI; CC₅₀/EC₅₀) ranged from >120 to >650. In particular, Compound 1 (AT-281) exhibited potent multi-serotypic anti-DENV activity in vitro. In Huh-7 cells infected with DENV-2 (New Guinea C strain), Compound 1 (AT-281) reduced the yield of infectious virus by 90% (EC₉₀) at a concentration of 0.64 μM. Similarly, in human PBMCs and BHK-21 cells infected with DENV2 (clinical isolate) and in Huh-7 cells infected with DENV-3 (H87 strain), Compound 1 (AT-281) exhibited respective EC₅₀ values of 0.60, 0.63 and 0.77 μM in inhibiting virus-induced CPE. The SI was >210 in each case.

To assess the antiviral specificity of Compound 1 (AT-281), serial dilutions were incubated with various host cell lines infected with a panel of DNA and RNA viruses other than flaviviruses. The activity of Compound 1 (AT-281) was measured in infected cells using the neutral red assay to determine the effective concentration required to inhibit viral replication 50% (EC₅₀) (described in Example 3) and the threshold concentration required to obtain a perceptible effect on 50% of the cells incubated without virus (CC₅₀). Compound 1 (AT-281) had no activity against the DNA viruses tested and was only weakly or not active against several RNA viruses (Table 2). However, it had a high selectivity (>2,000) towards HCV, inhibiting the virus at nanomolar levels (Table 2). The potential cytotoxicity of Compound 1 (AT-281) was assessed in multiple cell lines with resulting CC₅₀ values greater than 100 μM (Table 2, below).

TABLE 2
Antiviral selectivity of Compound 1 (AT-
281) against a panel of RNA and DNA viruses

| Virus | Type | Cell | EC₅₀ (μM) | CC₅₀ (μM) |
|---|---|---|---|---|
| Chikungunya | RNA | Huh-7 | 54.2 | >100 |
| Eastern equine encephalitis | RNA | Huh-7 | 10.2 | >100 |
| HCV | RNA | Huh-7 | 0.05 | >100 |
| HIV-1 | RNA | CEM-SS | >100 | >100 |
| Influenza A and B | RNA | MDCK | >100 | >100 |
| Respiratory syncytial virus | RNA | HEp2 | >100 | >100 |
| Rhinovirus | RNA | H1-HeLa | >100 | >100 |
| Rift Valley Fever | RNA | Huh-7 | >100 | >100 |
| Adenovirus | DNA | Vero | >100 | >100 |
| Herpes simplex-1 | DNA | Vero | >100 | >100 |

A further lack of cytotoxicity was demonstrated in human induced pluripotent stem cell (iPS) cardiomyocytes (described in Example 6) and in granulocyte macrophage (GM) and erythroid (E) human bone marrow progenitor cells (described in Example 7). When incubated with Compound 1 (AT-281), these cells also had CC₅₀ values greater than 100 μM, whereas the positive control compounds had the expected cytotoxic effects (CC₅₀ values of 7 μM for doxazosin with cardiomyocytes and 2 and 3 μM for AZT in the GM and E cell assays, respectively). Additionally, it has been previously shown that the active TP, Compound 5 (AT-9010), does not inhibit the in vitro enzyme activities of human cellular DNA-dependent DNA polymerases α, β or γ with estimated IC₅₀ values>100 μM, nor is it likely to affect mitochondrial integrity or inhibit human mitochondrial DNA-directed RNA polymerase (POLRMT; (Good et al., Preclinical evaluation of AT-527, a novel guanosine nucleotide prodrug with potent, pan-genotypic activity against hepatitis C virus. PLos One 15:e0227104).

### Example 3. Neutral Red Assay to Measure 50% Inhibition (EC₅₀) of the Virus-Induced Cytopathic Effect (CPE)

The neutral red assay procedure herein was used to generate the data marked “neutral red assay” or “NR” in Tables 1A and 1B of Example 2. AT-281 was dissolved in DMSO at a concentration of 10 mg/mL and serially diluted using eight half-log dilutions so that the highest test concentration was 100 μg/mL (172 μM). Each dilution was added to 5 wells of a 96-well plate with 80-100% confluent Huh-7 cells. Three wells of each dilution were infected with virus, and two wells remained uninfected as toxicity controls. Six untreated wells were infected as virus controls and six untreated wells were left uninfected to use as virus controls. Viruses were diluted to achieve MOIs of approximately 0.001 CCID₅₀ (50% cell culture infectious dose) per cell (0.037 and 0.028 CCID₅₀ for POWV LB strain and USUV, respectively). Plates were incubated at 37° C. in a humidified atmosphere containing 5% CO₂. On day 3 (ZIKV, EEEV and POWV Spooner), day 4 (CHIKV), day 5 (YFV, RVFV, MERS, POWV LB and USUV) or day 6 (WNV, JEV, DENV-2 NGC and DENV-3) post-infection, when untreated virus control wells reached maximum CPE, the plates were stained with neutral red dye for approximately 2 hours (±15 minutes). Supernatant dye was removed, wells were rinsed with PBS, and the incorporated dye was extracted in 50:50 Sorensen citrate buffer/ethanol for >30 minutes. The optical density was read on a spectrophotometer at 540 nm and converted to percent of controls. The concentrations of test compound required to prevent virus-induced CPE by 50% (EC₅₀) and to cause 50% cell death in the absence of virus (CC₅₀) were calculated (Smee et al., 2017. Evaluation of cell viability dyes in antiviral assays with RNA viruses that exhibit different cytopathogenic properties. J Virol Methods 246:51-57; Repetto et al., 2008. Neutral red uptake assay for the estimation of cell viability/cytotoxicity. Nature Protoc 3:1125-1131). The selective index is the CC₅₀ divided by EC₅₀ except where indicated. Selectivity index (SI) values between 0-3.9 indicate inactive compounds; SI of 4-9.9 indicates minimally active compounds, SI of 10-49.9 indicates moderately active compounds and SI values>50 indicate highly active compounds. Compounds with SI values>100 are indistinguishable from one another.

### Example 4. Virus Yield Reduction Assay to Measure the Concentration to Reduce Virus Yield by 1 Log₁₀ (EC₉₀)

The virus yield reduction assay procedure herein was used to generate the data marked “VYR” or “VYR assay” in Tables 1A and 1B of Example 2. As previously published (Prichard et al., 1990. A microtiter virus yield reduction assay for the evaluation of antiviral compounds against human cytomegalovirus and herpes simplex virus. J Virol Methods 246:51-57), Vero 76 cells were seeded in 96-well plates and grown overnight (37° C.) to confluence. A sample of the supernatant fluid from each compound concentration was collected on day 3 (day 4 for POWV and day 7 for USUV) post infection (3 wells pooled) and tested for virus titer using a standard endpoint dilution CCID₅₀ assay and titer calculations using the Reed-Muench equation (Reed et al., 1938. A simple method of estimating fifty percent endpoints. Am J Hygiene 27:493-497). The concentration of compound required to reduce virus yield by 90% (EC₉₀) was determined by regression analysis.

### Example 5. DENV-2 Infection and Treatment of Human PBMCs and BHK-21 Cells

The PBMC assay procedure herein was used to generate the data marked “PBMC” or “Human PBMC” in Tables 1A and 1B of Example 2. Compound 1 (AT-281) was dissolved in DMSO at 100 mM and then diluted in growth medium to final concentrations of 100, 20, 4 and 0.8 μM. PBMCs were resuspended in RPMI 1640 medium with 2 mM L-glutamine, 10% FBS, 100 U/mL penicillin and 100 μg/mL streptomycin (PBMC growth medium) and were subjected to a 3-day activation with PHA (5 μg/mL). Following a 1-h infection with DENV-2, the cells were seeded in 96-well plates (1×10⁵ cells/well), in PBMC growth medium, either in the absence or presence of serial dilutions of Compound 1 (AT-281). Similarly, BHK-21 cells were grown to confluency in 96-well plates, then growth medium was replaced with fresh maintenance medium (growth medium with 1% inactivated FBS in place of 10% FBS) containing serially diluted Compound 1 (AT-281) and DENV-2 at a multiplicity of infection (MOI) of 0.01. Uninfected cells in the presence of serially diluted compound were used to assess the cytotoxicity of compounds. After a 3-day incubation at 37° C. in a humidified 5% CO₂ atmosphere, cell viability was determined by the MTT method (Pauwels et al., 1988. Rapid and automated tetrazolium-based colorimetric assay for the detection of anti-HIV compounds. J Virol Methods 20:309-21). The effective concentration of AT-281 required to prevent virus-induced cytopathic effect (CPE) by 50% (EC₅₀) and to cause 50% cell death in the absence of virus (CC₅₀) were calculated by regression analysis.

### Example 6. Cardiomyocyte Assay to Determine Cytotoxic Concentration of the Drug to Cause Death to 50% of Viable Cells Incubated without Virus (CC50)

The cardiomyocyte cytotoxicity assay procedure herein was used to generate the CC₅₀ data in Table 2 of Example 2. Cytotoxicity was evaluated in human iPS cardiomyocytes (Cellular Dynamics; Madison, WI) and in primary human bone marrow progenitor cells (Invitrogen, Grand Island, NY). Doxazosin mesylate and AZT, used as positive controls respectively, were purchased from Sigma Aldrich. The iPS cardiomyocytes, in medium from Cellular Dynamics, were seeded in 96-well plates pre-coated with 0.1% gelatin (Sigma) at 1.5×10⁴ cells/well in a final volume of 100 μL, and incubated at 37° C. in 5% CO₂ for 48 h. The cells were washed with DPBS, and AT-281 diluted in the same medium (100 μL) was added to the monolayer in triplicate and incubated at 37° C. in 5% CO₂ for 3 days. Cell viability was measured by staining with CellTiter Glo®. The medium was removed from the test plates, and replaced with fresh medium (100 μL) and CellTiter Glo reagent (100 μL per well) before incubating at RT for 10 min. The well contents were transferred to a white 96-well plate and luminescence measured within 15 min on a Wallac 1450 Microbeta Trilux liquid scintillation counter.

### Example 7. Bone Marrow Progenitor Cell Assay

The cardiomyocyte cytotoxicity assay procedure herein was used to confirm the CC₅₀ data in Table 2 of Example 2. Bone marrow progenitor cells suspended in Iscove modified Dulbecco medium containing 15% heat-inactivated FBS, 10% giant cell tumor conditioned medium (Bone Marrow Plus, Sigma), 10 ng/mL recombinant human IL-6, 10 ng/mL recombinant human IL-3 and 25 ng/mL recombinant human granulocyte macrophage colony stimulating factor (GM-CSF, R&D systems), and a final concentration of 1% methylcellulose, were added to 6-well plates (1×10⁵ cells/well) in a volume of 900 μL. AT-281 (100 μL) at 10 times high test concentration were added to each well in triplicate, incubated at 37° C. in 5% CO₂ for 14 days, and colonies (greater than 30 cells) counted.

### Example 8. Compound 1 (AT-281) Forms the Active Metabolite Compound 5 (AT-9010) in Human PBMCs In Vitro

Since virus infection in the blood is an important component of Dengue fever, it was important to determine the stability of Compound 1 (AT-281) in blood, and the ability of PBMCs to phosphorylate Compound 1 (AT-281) to form the active triphosphate metabolite, Compound 5 (AT-9010). Compound 2 (AT-752) was found to be stable for up to 120 min in cynomolgus monkey and human plasma (98.2% and 93.5% of the respective starting 2 μM concentrations remained after 120 min of incubation), but was highly unstable in Sprague Dawley (SD) rat plasma with no drug remaining at the first time point (10 min of incubation). The half-life (T_{1/2}) in rat plasma at 37° C. was estimated to be <3 minutes.

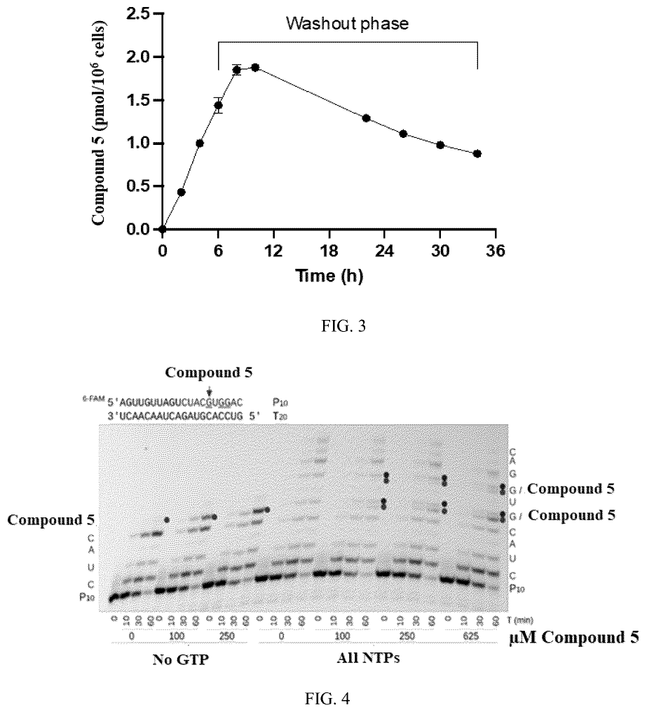

Fresh non-stimulated human PBMCs treated with 10 μM Compound 1 (AT-281) (as described in Example 9) for up to 6 h had a time-dependent increase in Compound 5 (AT-9010) concentrations over the entire exposure phase and continued to increase after drug washout, with a mean peak concentration of 1.88±0.01 pmol/10⁶ cells at 10 h after initiation of exposure (FIG. 3). When Compound 1 (AT-281) was removed for 28 h (wash-out phase), levels of Compound 5 (AT-9010) in the PBMCs decreased time-dependently (FIG. 3) with an estimated T_{1/2} of 21.6 h.

### Example 9. Formation of Compound 5 (AT-9010) In Vitro in Non-Stimulated Human PBMCs Treated with Compound 1 (AT-281)

Upon receipt, fresh human PBMCs (single male donor, Lot LS-88-45477C) were centrifuged at 400×g for 5 min. The resulting cell pellet was suspended in 20 mL of warm PBMC growth medium. The cell density of the cell suspension was counted using an automated cell counter (Cellometer K2, Nexcelom) after staining with Trypan Blue and adjusted to 2×10⁶ viable cells/mL. The human PBMC suspension was transferred to 24-well tissue culture treated plates at 0.5 mL/well (1×10⁶ cells) and cultured in a humidified incubator maintained at 37° C. and 5% CO₂ for 20-24 h. Then, Compound 1 (AT-281) stock solution was spiked into each well to achieve the final concentration of 10 μM, and the stimulated cells were incubated for 0, 2, 4 and 6 h at 37° C. and 5% CO₂ atmosphere. At each time point, samples in triplicate were processed for analysis of Compound 5 (AT-9010). For the washout samples, the incubated PBMCs were collected and centrifuged at 400×g for 5 min. The supernatant was discarded and the cells washed with 0.5 mL medium, re-suspended in 0.5 mL medium and transferred to individual wells of 24-well plates. The human PBMCs were further incubated in a humidified incubator maintained at 37° C. and 5% CO₂ for an additional 0, 2, 4, 16, 20, 24 and 28 h (i.e., 6, 8, 10, 22, 26, 30 and 34 h after the initiation of test article exposure). At each time point, triplicate samples were processed for Compound 5 (AT-9010) analysis as follows: Samples were transferred into 2 mL tubes and centrifuged at 800×g for 5 min. After aspirating the supernatant, each sample was vortexed after the addition of 0.1 mL water. They were then quenched with 0.3 mL ice-cold 60% MeOH, and stored at −70° C. until analyzed using LC/MS/MS (FIG. 3).

### Example 10. Compound 5 (AT-9010) Disrupts RNA Elongation by DENV NS5

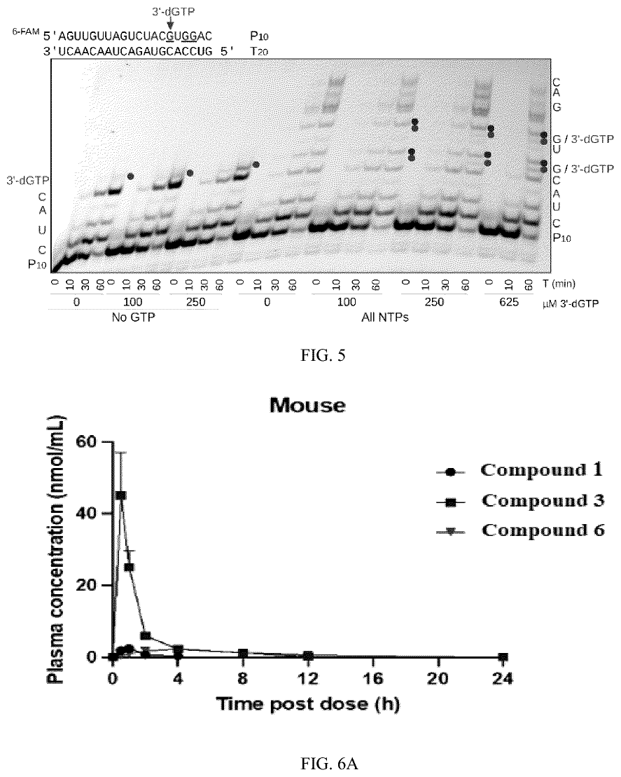

To confirm the mechanism of action and antiviral target of Compound 5 (AT-9010), incorporation and elongation assays (described in Example 13) were run using the full-length DENV NS5 protein (described in Example 11) (serotype 2) and an annealed primer-template RNA pair mimicking the 3′ end of the DENV-2 genome (described in Example 12). In the absence of GTP, Compound 5 (AT-9010) was readily incorporated as a substitute at the +5 position of the RNA primer (FIG. 4, left side of gel). Despite the presence of the next correct nucleotide (UTP), no further extension was observed, indicating that Compound 5 (AT-9010) causes immediate chain-termination of RNA synthesis irrespective of the presence of its 3′OH group. In the presence of all four NTPs, Compound 5 (AT-9010) competed with GTP for incorporation at +5, and +7 positions (FIG. 4, right side). In fact, incorporation of GTP and Compound 5 (AT-9010) at the same positions could be differentiated on the gel, and the discrimination factor (preference for GTP>Compound 5 (AT-9010), as judged by band product intensities) was calculated to be 12.6±4.3. Comparison of incorporation with the obligate chain terminator 3′-dGTP showed an equivalent profile, supporting chain termination as the mechanism of action of Compound 5 (AT-9010) (FIG. 5).

### Example 11. DENV NS5 Protein Expression and Purification

The DENV NS5 protein used in Example 10 was prepared by the procedure described herein. Full-length DENV NS5 (serotype 2, New Guinea C) was expressed and purified as previously described (Potisopon et al., 2017. Substrate selectivity of Dengue and Zika virus NS5 polymerase towards 2′-modified nucleotide analogues. Antiviral Res 140:25-36). Briefly, the gene coding for NS5 with a N-terminal 6-His tag was expressed from a pQE30 vector in E. coli NEB Express cells (New England Biolabs, Ipswich, MA) transformed with pRare2-LacI (Novagen, Madison, WI), and induced with 50 μM IPTG and 2% EtOH until the OD₆₀₀ value reached 0.6. Cells were lysed with sonication, and the NS5 protein separated using TALON metal-affinity resin slurry (Clontech, Mountain View, CA) according to the manufacturer's instructions, washed and eluted. Size exclusion chromatography was carried out as a second purification step where the protein was loaded onto a Superdex 200 HR 16/20 column (GE Healthcare), and eluted in a buffer containing 50 mM HEPES pH 7.5, 300 mM NaCl, 10% glycerol and 1 mM DTT.

### Example 12. Oligonucleotides and Nucleotides

The RNA oligonucleotides used in Example 10 was prepared as described herein. RNA oligonucleotides were purchased from Biomers.net (Ulm/Donau, Germany). A 20 nt template sequence (T₂₀) corresponding to the 3′ end of the DENV-2 antigenome was annealed to a 10 nt complementary primer (P₁₀) containing a fluorescent 6-FAM moiety at the 5′ end. Annealing was performed using a primer-template molar ratio of 1:1.5 in the presence of 110 mM KCl, incubated at 70° C. for 10 min and cooled slowly to room temperature. NTPs were purchased from GE Healthcare (Chicago, IL).

### Example 13. Nucleotide Analogue Incorporation Assay

DENV NS5 was preincubated with the annealed P₁₀/T₂₀ RNA in an assembly buffer containing 20 mM HEPES pH 7.5, 10% glycerol, 5 mM MgCl₂ and 5 mM DTT for 10 min at 30° C. to create an active RNA elongation complex. Reactions were started by adding AT-9010 with either all four NTPs, or UTP, ATP and CTP only. Final concentrations were 0.5 μM NS5, 0.25 μM P₁₀/T₂₀, 100 μM each NTP, and between 10-625 μM AT-9010, in a final buffer containing 20 mM HEPES pH 7.5, 15% glycerol, 5 mM MgCl₂ and 5 mM DTT. Reactions were quenched at indicated timepoints in 2× volume FBD stop solution (formamide, 10 mM EDTA) and analysed on 20% acrylamide:bisacrylamide (19:1), 7 μM urea sequencing gels. RNA products were visualized using a Typhoon FluorImager and analyzed using ImageQuant software.

### Example 14. Compound 2 (AT-752) Demonstrates Favorable Pharmacokinetics in Preclinical Species

The plasma pharmacokinetics (PK) of Compound 1 (AT-281) and its metabolites Compound 3 (AT-551), the intermediate prodrug, and Compound 6 (AT-273), the plasma surrogate for intracellular levels of the active triphosphate metabolite Compound 5 (AT-9010), were determined in CD-1 mice, SD rats and cynomolgus monkeys after single oral doses of Compound 2 (AT-752) at 420, 300 and 300 mg/kg, respectively (Table 3).

TABLE 3
Pharmacokinetic parameters following administration
of a single oral dose of AT-752

| | | Dose | Cₘₐₓ^{a} | Tₘₐₓ^{b} | AUC_{0-24 h}^{c} |
|---|---|---|---|---|---|
| Metabolite | Species | (mg/kg) | (nmol/mL) | (h) | (nmol*h/mL) |
| Compound 1 | Mouse | 420 | 2.6 ± 0.3 | 0.8 ± 0.3 | 4.2 ± 0.5 |
| | Rat | 300 | nd | nd | nd |
| | Monkey | 300 | 3.7 ± 1.5 | 1.0 ± 0.0 | 9.5 ± 3.7 |
| Compound 3 | Mouse | 420 | 45.1 ± 12.0 | 0.5 ± 0.0 | 58.7 ± 13.3 |
| | Rat | 300 | 16.5 ± 10.8 | 1.0 ± 0.0 | 56.5 |
| | Monkey | 300 | 2.0 ± 0.8 | 2.0 ± 0.0 | 9.7 ± 2.2 |
| Compound 6 | Mouse | 420 | 2.3 ± 0.7 | 4.0 ± 0.0 | 18.8 ± 6.0 |
| | Rat | 300 | 1.3 ± 0.3 | 8.0 ± 0.0 | 22.9 |
| | Monkey | 300 | 0.8 ± 0.2 | 4.0 ± 0.0 | 7.8 ± 2.4 |

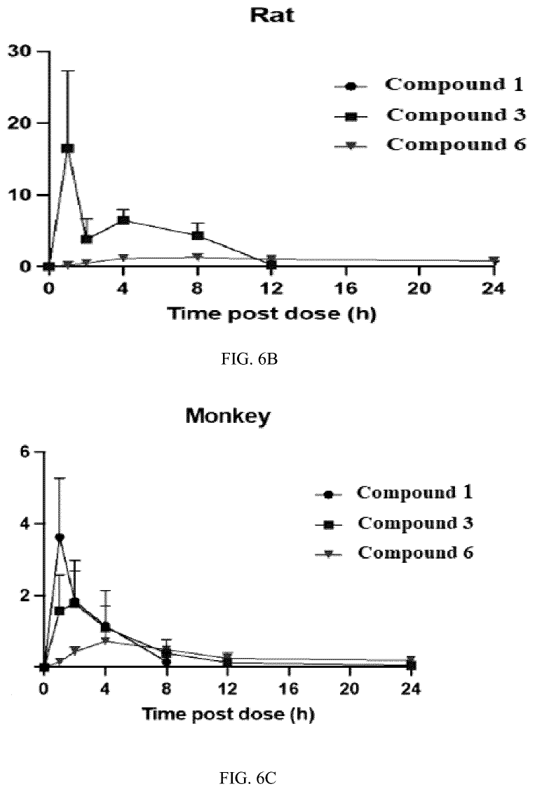

In rodents, the parent prodrug Compound 1 (AT-281) was quickly metabolized with the rapid appearance in plasma of its metabolites, Compound 3 (AT-551) and Compound 6 (AT-273) (FIGS. 6A and 6B). For rats, the plasma concentration of Compound 1 (AT-281) was below the limit of quantitation at 1 h (FIG. 6B), the earliest time point measured, which is consistent with the metabolic instability of Compound 1 (AT-281) in rat plasma reported above. In monkeys, Compound 1 (AT-281) was present for more than 4 h in plasma (FIG. 6C), with a Cₘₐₓ of 3.7±1.5 nmol/mL at 1 h. The prodrug was converted to intermediate plasma metabolites Compound 3 (AT-551) and Compound 6 (AT-273), with PK comparable to previously reported results of its congener AT-511 (Good et al., Preclinical evaluation of AT-527, a novel guanosine nucleotide prodrug with potent, pan-genotypic activity against hepatitis C virus. PLos One 15:e0227104).

### Example 15. The Active Metabolite Compound 5 (AT-9010) is Formed in PBMCs In Vivo

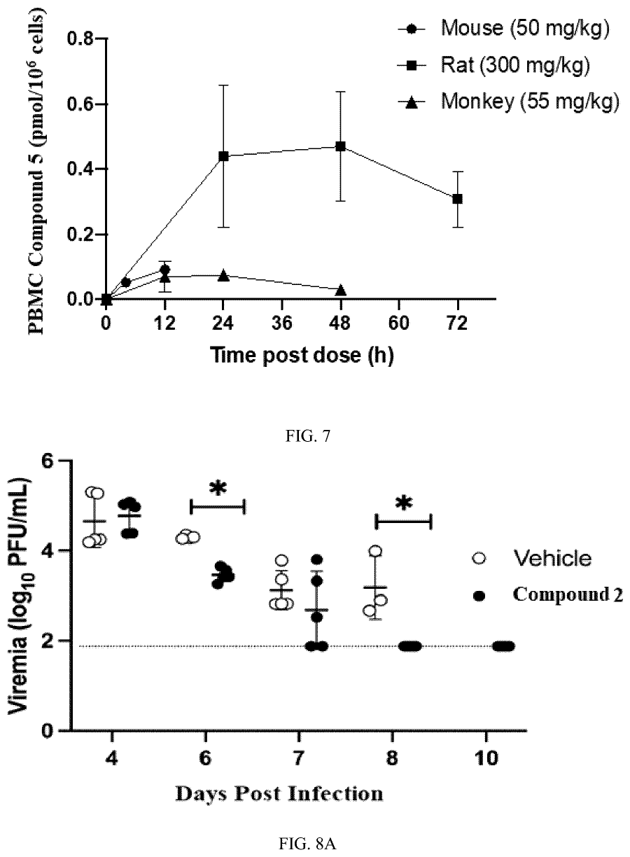

Concentrations of Compound 5 (AT-9010) in PBMCs isolated from mice and rats administered single oral doses of Compound 1 (AT-281) at 50 and 300 mg/kg and monkeys after a single oral dose of Compound 2 (AT-752) equivalent to 55 mg/kg Compound 1 (AT-281) were measured at various time points post dose (FIG. 7). After comparable doses, similar concentrations of Compound 5 (AT-9010) were observed in mice and monkeys at 12 h (0.092 and 0.070±0.046 pmol/10⁶ cells). At a 6-fold higher dose, PBMCs from rats at 24 h exhibited 5- to 6-fold more Compound 5 (AT-9010) (0.439±0.218 pmol/10⁶ cells) than the 12-h values observed in mice and monkeys, suggesting a similar extent of active TP formation in PBMCs across all three species, despite the very low exposure to Compound 1 (AT-281) in the systemic circulation of rats and, to a lesser extent, mice. When mice were given a loading dose of 500 mg/kg Compound 1 (AT-281), followed by a second dose of 250 mg/kg 4 h later, the concentrations of Compound 5 (AT-9010) in PBMCs at 4 and 12 h after the second dose were 0.297 and 0.322 pmol/10⁶ cells, respectively (not shown).

To test the tolerability of Compound 1 (AT-281) and determine steady state concentrations of the active TP in PBMCs, mice were given a loading dose of 1000 mg/kg and, starting 4 h later, dosed twice a day (12 h apart) at 500 mg/kg for 3 days. The concentrations of Compound 5 (AT-9010) in pooled (n=3) PBMCs 12 h after the first and sixth 500 mg/kg dose were 0.421 and 0.575 pmol/10⁶ cells, respectively, while the concentrations of plasma Compound 6 (AT-273) were 0.67±0.12 and 1.35±0.86 nmol/mL, respectively, for the same time points. There were no adverse clinical signs observed in the mice so the multiple doses of the prodrug were well-tolerated.

### Example 16. Animal Welfare

The studies using CD-1 mice, SD rats and cynomolgus monkeys in Example 17 were conducted at WuXi AppTec (Suzhou, China) in strict compliance with AAALAC International, NIH guidelines and the People's Republic of China, Ministry of Science and Technology, “Regulations for the Administration of Affairs Concerning Experimental Animals”, 2017. Protocols were reviewed and approved by WuXi AppTec's IACUC prior to study initiation, and all animals assessed by the WuXi AppTec veterinary staff throughout the studies. All animals were housed in rooms with controlled temperature (18 to 26° C.), relative humidity (40 to 70%) and light cycle (12 h artificial light and 12 h dark), with 100% airflow. They were provided with manipulatives/enrichment toys. The AG129 mouse study described herein was conducted at IBT Bioservices (Rockville, MD) in strict compliance with USDA Animal Welfare Act and follow the PHS and NIH Policy of Humane Care and Use of Laboratory Animals, and the Guide for the Care and Use of Laboratory Animals, National Research Council—ILAR, Revised 2011. The study was conducted in full compliance with protocols that were reviewed and approved by IBT's Institutional Animal Care and Use Committee (IACUC) prior to study initiation, and mice were assessed and monitored throughout the study by members of the IBT veterinary staff in accordance with PHS Policy and the USDA.

### Example 17. PK Studies and In Vivo Formation of Compound 5 (AT-9010) in Mice, Rats and Monkeys

Male naïve CD-1 mice (Shanghai Sippr BK Laboratory Animals Co. Ltd., China) were administered a dose of Compound 2 (AT-752) at 420 mg/kg in 40% PEG400, 10% solutol HS15, 50% 100 mM citrate buffer, pH 4.5 (v/v) by oral gavage. Blood samples (N=3) were collected at 0.5, 1, 2, 4, 8, 12 and 24 h post-dose, plasma separated in EDTA and 1 μL dichlorvos (2 mg/mL; stabilizing agent to prevent in vitro hydrolysis of the ester moiety of Compound 1 (AT-281) by blood esterases) and stored at −70° C. Concentrations of Compound 1 (AT-281), and metabolites Compound 3 (AT-551) and Compound 6 (AT-273) were determined in the plasma by LC/MS/MS. For PBMCs, six male naïve CD-1 mice were administered a dose of Compound 2 (AT-752) (at an Compound 1 (AT-281) equivalent dose of 50 mg/kg) in 60% PEG400 by oral gavage. Blood samples were collected, in groups of three, at 4 and 12 h post-dose, PBMCs isolated, and concentrations of Compound 5 (AT-9010) were measured by LC/MS/MS.

Male naïve SD rats (Beijing Vital River Laboratory Animals Co. Ltd., China) were administered a dose of AT-752 at 300 mg/kg in 40% PEG400, 10% solutol HS15, 50% 100 mM citrate buffer, pH 4.5 (v/v) by oral gavage. Blood samples (n=4) were collected at 1, 2, 4, 8, 12 and 24 h post-dose, plasma separated and stored at −70° C. Concentrations of Compound 1 (AT-281), and metabolites Compound 3 (AT-551) and Compound 6 (AT-273) were determined by LC/MS/MS. For PBMCs, nine male naïve SD rats were administered a dose of Compound 2 (AT-752) (at a Compound 1 (AT-281) equivalent dose of 300 mg/kg) by oral gavage. Blood samples were collected, in groups of three, at 24, 48 and 72 h post-dose, PBMCs isolated, and Compound 5 (AT-9010) concentrations determined by LC/MS/MS.

Male non-naïve cynomolgus monkeys (Hainan Jingang Laboratory Animal Co. Ltd., China), at least 2 kg body weight, were individually housed during the study in stainless steel mesh cages, provided ad libitum access to RO water, fed twice daily (except on the day of dosing when they were fed once) with ˜60 g Certified Monkey Diet each feed (Beijing Vital Keao Feed Co., Ltd., Beijing, China) and given daily treats of fresh fruit. They (n=5) were administered a dose of Compound 2 (AT-752) at 300 mg/kg in 40% PEG400, 10% solutol HS15, 50% 100 mM citrate buffer, pH 4.5 (v/v) by oral gavage, and blood samples (˜0.5 mL) were collected at 1, 2, 4, 8, 12 and 24 h post dose. Plasma was separated at each time point for pharmacokinetics, and stored at −70° C. Concentrations of Compound 1 (AT-281), and metabolites Compound 3 (AT-551) and Compound 6 (AT-273) were determined by LC/MS/MS. Separately, three monkeys were given an oral dose of Compound 2 (AT-752) as powder in capsules at 60 mg/kg (Compound 2 (AT-281) equivalent dose of 55 mg/kg), and blood samples collected at 12, 24 and 48 h post dose. PBMCs were isolated and concentrations of Compound 5 (AT-9010) measured by LC/MS/MS. All animals were observed for any unusual or adverse clinical signs just before and immediately after dosing and prior to each blood collection time point, with no such signs noted.

Separately, two mouse studies with multiple dosing were conducted. First, 9 naïve CD-1 mice were administered Compound 1 (AT-281) in 100 mM citrate buffer, pH 4.5 by oral gavage at 500 mg/kg for the first dose and, 4 h later, 250 mg/kg for the second dose. Blood samples were collected, 3 animals per time point, at 4 (before the second dosing), 8 and 16 h post the first dose, and PBMCs isolated. Secondly, 9 naïve CD-1 mice were administered Compound 1 (AT-281) by oral gavage at 1000 mg/kg for the first dose, and then 4 h later, they were given twice daily doses 12 hours apart for 3 consecutive days at 500 mg/kg. Blood samples were collected, 3 animals per time point, at 16 (received one 500 mg/kg dose), 40 (received three 500 mg/kg doses) and 76 h post first dose (received six 500 mg/kg doses), and PBMCs were isolated. Concentrations of Compound 1 (AT-281) and Compound 6 (AT-273) were determined in the plasma, and Compound 5 (AT-9010) in the PBMCs by LC/MS/MS (described in Example 21).

### Example 18. Compound 2 (AT-752) Reduces Viremia and Improves Survival of AG129 Mice after DENV2 D2Y98P Infection

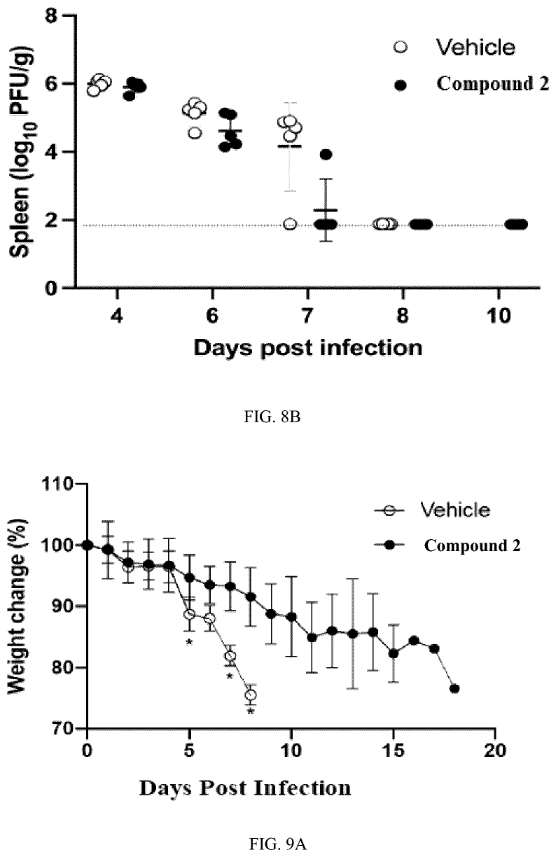

To test the efficacy of Compound 2 (AT-752) against DENV infection, AG129 mice were subcutaneously inoculated with DENV-2 D2Y98P, a non-mouse-adapted strain that follows a similar disease kinetic as described in humans (Tan et al., 2010. A non mouse-adapted Dengue virus strain as a new model of severe Dengue infection in AG129 mice. PLoS Negl Trop Dis 4:3672). The prodrug was orally administered as a loading dose (1000 mg/kg) 4 h prior to viral challenge, and afterwards as twice a day (BID) doses (500 mg/kg) for 7 consecutive days, beginning 1 h post infection (pi). Viremia and spleen viral load, the primary endpoints, were evaluated at Days 4, 6, 7, 8 and 10 pi. Viremia (FIG. 8A) in the Compound 2 (AT-752) treated group was significantly lower than the vehicle only group on Day 6 pi (p=0.000014 by t-test) and was cleared in all treated animals by Day 8 pi whereas virus was still measurable in all surviving controls (p=0.0045 by t-test). Although Compound 2 (AT-752) treatment reduced the spleen viral load by Day 7 pi compared to the control group (FIG. 8B), this change was not statistically different between the groups. This result is likely because virus is eventually cleared from spleen in this mouse model of disease.

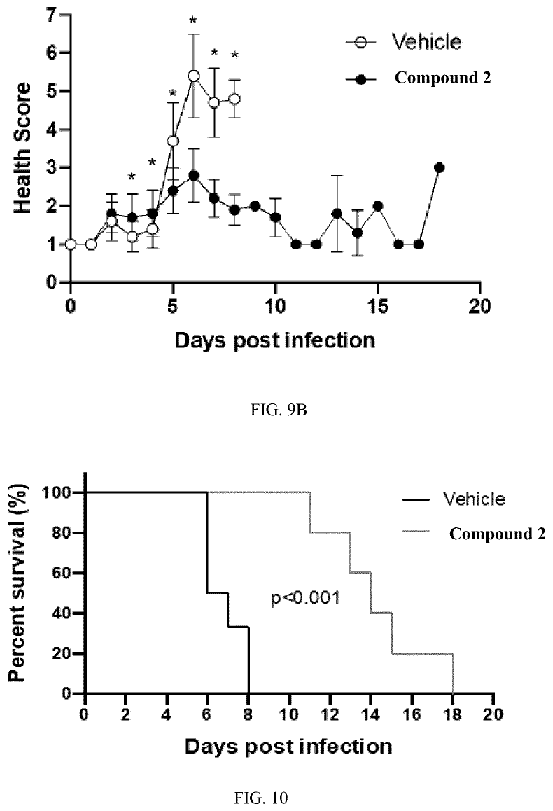

Mice were evaluated daily for weight change, appearance, mobility and alertness, and euthanized when 20% weight loss or a health score >5 was reached. There were notable differences in body weight between the treated and vehicle control groups at Days 4 to Day 8 pi (p<0.001 by t-test). No vehicle-treated mice survived beyond Day 8 pi. Animals treated with Compound 2 (AT-752) had significantly reduced weight loss compared to vehicle control mice on Days 5, 7 and 8 pi (FIG. 9A; p<0.001 by t-test). In addition, mice treated with Compound 2 (AT-752) had significantly better health scores than the vehicle control group on Day 4 to Day 9 pi (FIG. 9B; p<0.001 by t-test). The resulting mortality data, presented as Kaplan-Meier survival curves (FIG. 10), demonstrate that Compound 2 (AT-752) significantly improved the survival of the infected mice compared to the vehicle control group (p=0.0004, Mantel-Cox log-rank test). All mice in the control group not sacrificed as scheduled for collection of blood and spleen samples died or were euthanized on Days 5 to 8 pi. In contrast, those animals treated with Compound 2 (AT-752) survived to between Days 11 and 18 pi.

### Example 19. AG129 Mouse Study

The tissue samples used in Example 20 were collected according to the protocol described herein. Fifty-five female AG129 mice (α-, β-, γ-interferon knockout), 6-8 weeks old, were divided into two groups, with 25 mice in Group 1 (vehicle) and 30 mice in Group 2. On Day 0, all mice were challenged with 1×10⁵ PFU of DENV-2 D2Y98P by subcutaneous injection. Group 1 animals received vehicle [(PEG400 (40%, v/v)/Solutol HS15 (10%, v/v)/100 mM Citrate buffer pH 4.5±0.2 (50%, v/v)] by oral gavage BID for 7 consecutive days starting at 1 h pi. Group 2 received a single dose of 1000 mg/kg of Compound 2 (AT-752), 4 h prior to challenge, followed by a 500 mg/kg dose 1 h post challenge (pi). BID dosing of Compound 2 (AT-752) at 500 mg/kg was continued for 7 consecutive days. The protocol called for five mice per group to be sacrificed and terminal sera and spleens to be collected on Days 4, 6, 7, 8 and 10 pi. On Day 20 pi, any surviving mice in Group 2 were sacrificed for terminal sera and spleen collection. Serum samples were stored at −80° C. until further analysis. Harvested spleen samples were weighed and flash-frozen in a mix of ethanol/dry ice and stored immediately at −80° C. All samples were processed and assayed for viral load via plaque assay. All animals were monitored daily for weight loss, morbidity, mortality, and neurological decay. Mice displaying severe illness as determined by >20% weight loss, a health score of greater than 5 (rating coat appearance, mobility and alertness), extreme lethargy, and/or paralysis were euthanized and harvested for terminal sera and spleens. Any mouse that was found dead, was harvested for spleen collection only.

### Example 20. Plaque Assay for Spleen Viral Load Determination

Spleen samples collected according to Example 19 were homogenized in 250 μL DPBS using a TissueRuptor. Homogenates were then centrifuged and the supernatants collected and frozen immediately in two different aliquots until the plaque assay was performed. The results of the plaque assay are shown in FIGS. 8A and 8B.

On the day prior to the assay, Vero cells were seeded at a density of 1×10⁵ cells/well in 1 mL 1% Hi-FBS medium in 24-well tissue culture plates and incubated overnight at 37° C. in a 5% CO₂ atmosphere. The following day, 10-fold serial dilutions (10⁻² to 10⁻⁵) of the serum and spleen samples were prepared for titration in triplicate. Cell culture medium from the Vero cells was removed and 100 μL of MEM medium supplemented with 2 mM L-glutamine and 1× Pen/Strep was added along with 100 μL of the diluted samples. Cells were incubated for 1 h at 37° C. in a 5% CO₂ atmosphere. Then, 1 mL 0.8% methylcellulose containing 2% FBS supplemented with 2 mM L-glutamine and 1× Pen/Strep was added to the cells without inoculum removal and incubated for an additional 3 days at 37° C. in a 5% CO₂ atmosphere to allow plaque formation. Cells were fixed and permeabilized using a cold 80% ethanol/20% methanol mixture for 30 minutes at −20° C. 200 L anti-Dengue monoclonal antibody diluted 1:2000 in 5% nonfat milk was added to each well and incubated overnight at 4° C. Wells were washed 3 times with 1×DPBS, 200 μL of HRP-conjugated goat anti-mouse antibody at 1:2000 dilution in 5% non-fat milk was added, and the wells incubated for 1 h at room temperature. Viral plaques were resolved and counted using insoluble peroxidase substrate (TrueBlue) and a plaque counter. Data for the spleen viral load was normalized to the spleen weight for each individual mouse.

### Example 21. LC-MS/MS Analysis of Compound 1 (AT-281), Compound 3 (AT-551), Compound 6 (AT-273) and Compound 5 (AT-9010)

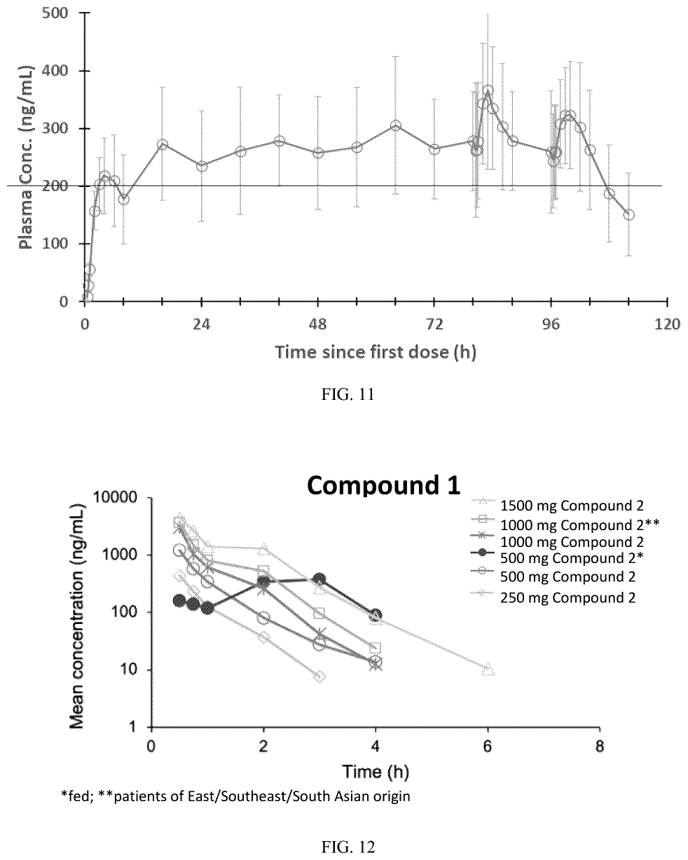

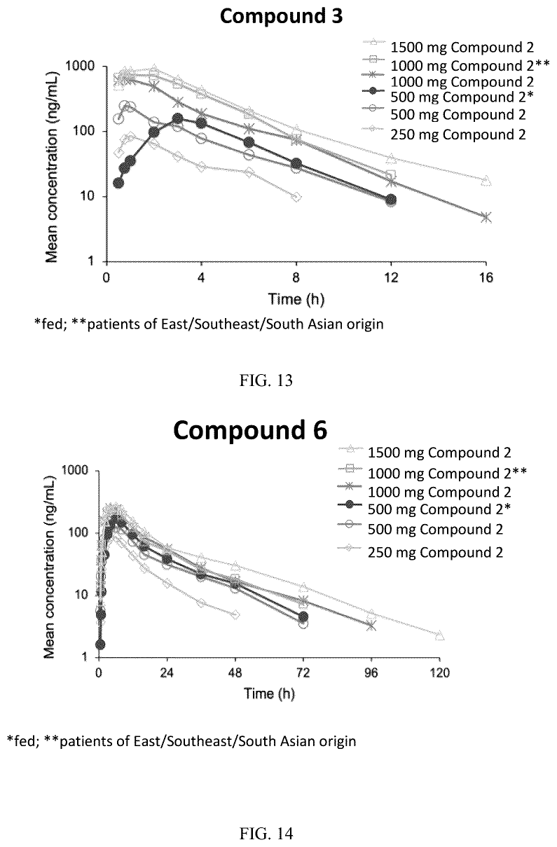

Pharmacokinetic measurements for FIGS. 11, 12, 13, 14, and 15 were analyzed using LC-MS/MS according to the protocol described herein. Plasma samples were prepared for MS analysis by adding internal standards and extracting with 20 volumes chilled ACN. After vortex (800 rpm for 10 min) and centrifugation (4,000 rpm, 15 min, 4° C.), the supernatants (25 μL) were diluted with an equal volume of H₂O, mixed and spun again. After adding internal standards, the PBMC samples (10⁶ cells) were lyzed with 300 μL MeOH/H₂O (70:30, v/v) and 40 mM dibutylammonium acetate (DBAA), mixed by vortex and centrifuged (12,000 rpm, 15 min, 4° C.). Aliquots of the supernatants (35 μL) were dried under nitrogen and reconstituted in H₂O, then mixed and spun again. To measure Compound 1 (AT-281) and plasma metabolites Compound 3 (AT-551) and Compound 6 (AT-273), 4 μL samples were injected onto an Acquity Gemini C₁₈ (50×4.6 mm), 5 μm UPLC column with a Sciex Triple Quad 6500 mass spectrometer (ESI positive ion, MRM mode). A binary nonlinear gradient with mobile phases A (0.1% formic acid in water) and B (0.1% formic acid in ACN) were used to elute samples at 0.8 mL/min, with a run time of 5 min. For AT-9010 in cells, 8 μL was injected onto an Acquity BEH C18 (50×2.1 mm), 1.7 μm UPLC column with a API 14000 mass spectrometer (ESI negative ion, MRM mode). A binary nonlinear gradient with mobile phases A (0.001% NHr H₂O, 0.18 mM DBAA in H₂O) and B (10 mM N,N-dimethyl-hexylamine, 3 mM NH₄OAc in ACN/H₂O (50.50, v/v)) were used to elute samples at 0.5 mL/min, with a run time of 3 min. Standards in 50% MeOH were used for calibration. Ions monitored were m/z 538.2/440.0 (AT-9010), 582.3/330.1 (AT-281), 464.2/165.1 (AT-551) and 300.2/152.2 (AT-273). Internal standards (ISS) as described previously (Good et al., Preclinical evaluation of AT-527, a novel guanosine nucleotide prodrug with potent, pan-genotypic activity against hepatitis C virus. PLos One 15:e02271045) were used to correct for variations in recovery.

### Example 22. Pharmacokinetic Data Analysis

Data analysis of the pharmacokinetic data generated according to Example 21 and presented in FIGS. 11, 12, 13, 14, and 15 was analyzed according to the procedure described herein. Plasma concentrations of Compound 1 (AT-281), Compound 3 (AT-551) and Compound 6 (AT-273) were subjected to non-compartmental pharmacokinetic analysis using Phoenix WinNonlin software (version 6.3 or above, Pharsight, Mountain View, CA). The linear/log trapezoidal rule was applied in obtaining the PK parameters.

### Example 23. Safety Study in Humans

Healthy males and females 18-65 years of age were enrolled in single ascending dose (SAD) and multiple ascending dose (MAD) cohorts and randomized to receive oral Compound 2 or placebo according to Tables 4-7.

Safety assessments including adverse events, standard clinical laboratory tests, vital sign measurements, electrocardiograms, and physical examinations were performed.

TABLE 4
Patients Enrolled in Study

| | SAD Cohorts | MAD Cohorts |
|---|---|---|
| Mean Age | 28.6 | 35.3 |
| Gender | 51.2% male, 48.8% female | 62.5% male, 37.5% female |
| Weight (kg) | 67.3 | 74.7 |

TABLE 5
Distribution of patients among cohorts in the study

| SAD Cohorts | Patient number | MAD Cohort | Patient number |
|---|---|---|---|
| A/B/C/D/E | 31 | F/G/H | 18 |
| Placebo | 10 | Placebo | 6 |

TABLE 6
Study Outline for SAD Cohorts

| Cohort | Does (mg) | Administration |
|---|---|---|
| A | 250 | Day 1 (fasting) |
| B (Food Effect) | 500 | Day 1 (fasting); Day 7 (fed)* |
| C | 1000 | Day 1 (fasting) |
| D (Ethnic)** | 1000 | Day 1 (fasting) |
| E | 1500 | Day 1 (fasting) |

*high-fat/high-calorie test meal.
**healthy participants of South/Southeast/East Asian origin

TABLE 7
Study Outline for MAD Cohorts

| Cohort | Dose (mg) | Administration |
|---|---|---|
| F | 1000 | QD × 7 days |
| G | 750 | BID × 4 days plus one dose Day 5 |
| H | 750 | TID × 4 days plus one dose Day 5 |

Compound 2 was well tolerated and no serious adverse events or discontinuations due to adverse events were reported. Nonserious adverse events were mild or moderate in severity and resolved by the end of the study (Table 8). Sporadic cases of gastrointestinal related events including mild to moderate vomiting occurred mostly at higher doses.

TABLE 8
Adverse events in SAD cohorts reported by more than two subjects

| Dose | Placebo | 250 | 500 | 500* | 1000 | 1000** | 1500 | Total |
|---|---|---|---|---|---|---|---|---|
| Number of | 10 | 6 | 7 | 7 | 6 | 6 | 6 | 41 |
| patients | | | | | | | | |
| Headache | 1 | 0 | 2 | 4 | 1 | 0 | 2 | 8 |
| Nausea | 0 | 0 | 0 | 1 | 1 | 0 | 1 | 3 |
| Vomiting | 0 | 0 | 0 | 0 | 1 | 0 | 2 | 3 |

*fed;
**patients of South/Southeast/East Asian origin

TABLE 9
Adverse events in MAD cohorts reported
by more than two subjects

| | | 1000 | 750 | 750 | | SAD and MAD |
|---|---|---|---|---|---|---|
| Dose | Placebo | QD | BID | TID | Total | Combined Total |
| Number of | 6 | 6 | 6 | 6 | 24 | 65 |
| patients | | | | | | |
| Headache | 1 | 0 | 1 | 0 | 2 | 10 |
| Nausea | 1 | 0 | 1 | 0 | 2 | 5 |
| Vomiting | 0 | 0 | 1 | 0 | 1 | 4 |

### Example 24. Pharmacokinetic Study in SAD Cohorts

Healthy males and females 18-65 years of age were enrolled in single ascending dose (SAD) and randomized to receive oral Compound 2 or placebo according to Tables 4-6.

Plasma and urine samples were collected at pre-determined timepoints and quantitated for Compound 1 and its metabolites Compounds 3, 6, and 7 using LC-MS/MS. Pharmacokinetic analyses were performed using non-compartmental approaches.

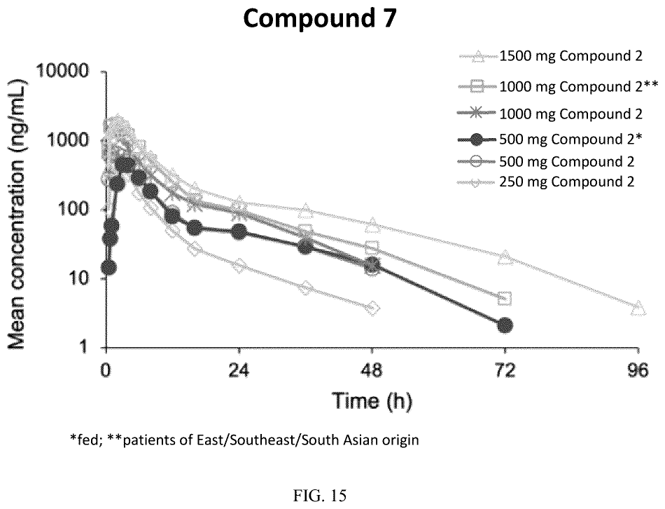

Upon oral dosing under fasting conditions, the parent prodrug Compound 1 was rapidly absorbed and cleared, followed by the appearance of Compound 3 which also exhibited a transient exposure (Tables 10-14, and FIG. 12-15). The N⁶-methyl 2′-methyl, 2′ fluoro nucleoside metabolite Compound 7 subsequently peaked and exhibited a slower elimination phase (FIG. 15). Compound 6 appeared more gradually in plasma and exhibited a long elimination half-life (cohort mean up to 23 hours), reflecting sustained intracellular exposure of the active triphosphate metabolite Compound 5.

### Dose Proportionality Analysis

Plasma exposure of Compound 1 and its metabolites increased over the studied dose range from 250 to 1500 mg. Compound 7 increased in a dose-proportional manner. Compound 1 and Compound 3 increased in a greater than dose-proportional manner whereas Compound 6 was slightly less than dose-proportional.

### Food Effect

A high-fat/high-calorie meal delayed and decreased peak level of Compound 1, Compound 3, and Compound 7, but had limited to no impact on their total exposure and slightly increased the plasma exposure of Compound 6.

TABLE 10
Summary of plasma pharmacokinetic parameters for Compound 1

| Dose | | Cₘₐₓ (ng/mL) | Tₘₐₓ (h) | AUC_{Inf} (ng/mL*h) | T_{1/2} (h) | CL_{R} (L/h) |
|---|---|---|---|---|---|---|
| 250 | mg | 482 ± 131 | 0.5 (0.5-0.8) | 347 ± 64 | 0.5 ± 0.1 | 7.5 ± 1.5 |
| 500 | mg | 1230 ± 1110 | 0.5 (0.5-6.0) | 1110 ± 687 | 0.5 ± 1.0 | 10.9 ± 5.4 |
| 500 | mg* | 488 ± 303 | 2.0 (0.5-3.0) | 980 ± 716 | 0.7 ± 0.3 | 6.6 ± 1.6 |
| Food effect | | 52.2 (22.1-123.6) | | 80.2 (38.7-166.2) | | |
| 1000 | mg | 3040 ± 960 | 0.5 (0.5-0.6) | 1915 ± 508 | 0.5 ± 0.1 | 11.4 ± 1.7 |
| 1000 | mg** | 3670 ± 1260 | 0.5 (0.5-0.5) | 2730 ± 944 | 0.5 ± 0.1 | 9.7 ± 4.2 |
| 1500 | mg | 5050 ± 2080 | 0.5 (0.5-0.8) | 4630 ± 1500 | 0.6 ± 0.2 | 8.3 ± 2.2 |

*fed;
**patients of South/Southeast/East Asian origin

TABLE 11
Summary of plasma pharmacokinetic parameters for Compound 3

| Dose | | Cₘₐₓ (ng/mL) | Tₘₐₓ (h) | AUC_{Inf} (ng/mL*h) | T_{1/2} (h) | CL_{R} (L/h) |
|---|---|---|---|---|---|---|
| 250 | mg | 89 ± 36 | 1.0 (0.8-2.0) | 302 ± 71 | 2.1 ± 0.3 | 2.0 ± 0.3 |
| 500 | mg | 231 ± 155 | 1.0 (0.8-8.0) | 898 ± 437 | 2.4 ± 0.5 | 2.1 ± 0.6 |
| 500 | mg* | 154 ± 84 | 3.1 (3.0-4.0) | 737 ± 289 | 2.3 ± 0.4 | 2.1 ± 0.4 |
| Food effect | | 70.5 (42.8-116) | | 78.3 (61.8-99.2) | | |
| 1000 | mg | 695 ± 468 | 0.8 (0.5-2.0) | 2330 ± 1060 | 2.2 ± 0.4 | 2.1 ± 0.6 |
| 1000 | mg** | 895 ± 418 | 0.9 (0.5-2.0) | 3320 ± 1630 | 2.0 ± 0.3 | 2.1 ± 0.6 |
| 1500 | mg | 1080 ± 217 | 1.5 (0.8-2.0) | 4100 ± 494 | 3.2 ± 1.2 | 1.9 ± 0.4 |

*fed;
**patients of South/Southeast/East Asian origin

TABLE 12
Summary of plasma pharmacokinetic parameters for Compound 6

| Dose | | Cₘₐₓ (ng/mL) | Tₘₐₓ (h) | AUC_{Inf} (ng/mL*h) | T_{1/2} (h) | CL_{R} (L/h) |
|---|---|---|---|---|---|---|
| 250 | mg | 91 ± 19 | 6.0 (3.0-6.0) | 1440 ± 280 | 19.1 ± 10.2 | 15.8 ± 2.8 |
| 500 | mg | 135 ± 25 | 4.0 (3.0-8.0) | 2390 ± 478 | 13.9 ± 5.9 | 16.8 ± 7.6 |
| 500 | mg* | 175 ± 29 | 6.0 (6.0-8.1) | 2990 ± 558 | 17.3 ± 11.0 | 13.4 ± 3.3 |
| Food effect | | 132 (109.8-158.6) | | 125.6 (111.2-141.8) | | |
| 1000 | mg | 241 ± 71 | 4.1 (4.0-6.3) | 4350 ± 1520 | 26.2 ± 12.2 | 17.1 ± 6.5 |
| 1000 | mg** | 240 ± 46 | 5.0 (3.1-6.0) | 3830 ± 519 | 16.3 ± 3.0 | 18.7 ± 2.1 |
| 1500 | mg | 283 ± 32 | 6.0 (3.2-6.1) | 5460 ± 1190 | 21.7 ± 6.2 | 19.5 ± 4.1 |

*fed;
**patients of South/Southeast/East Asian origin

TABLE 13
Summary of plasma pharmacokinetic parameters for Compound 7

| Dose | | Cₘₐₓ (ng/mL) | Tₘₐₓ (h) | AUC_{Inf} (ng/mL*h) | T_{1/2} (h) | CL_{R} (L/h) |
|---|---|---|---|---|---|---|
| 250 | mg | 446 ± 121 | 2.1 (2.0-3.0) | 2890 ± 680 | 16.9 ± 8.6 | 13.7 ± 1.6 |
| 500 | mg | 765 ± 307 | 1.0 (0.8-4.0) | 5480 ± 1630 | 15.1 ± 6.2 | 13.6 ± 5.4 |
| 500 | mg* | 488 ± 324 | 4.0 (3.0-6.1) | 4310 ± 1400 | 11.2 ± 4.9 | 12.5 ± 2.8 |
| Food effect | | 64.9 (43.8-96.2) | | 78.4 (68.4-89.9) | | |
| 1000 | mg | 1640 ± 474 | 0.9 (0.8-3.0) | 10300 ± 3070 | 12.4 ± 10.7 | 14.2 ± 3.8 |
| 1000 | mg** | 2010 ± 489 | 1.5 (0.8-2.0) | 14000 ± 4210 | 14.7 ± 10.1 | 13.4 ± 2.5 |
| 1500 | mg | 2030 ± 600 | 2.0 (1.0-2.0) | 16700 ± 3780 | 12.5 ± 3.8 | 15.0 ± 2.4 |

*fed;
**patients of South/Southeast/East Asian origin

Pharmacokinetic profiles were similar between the South/Southeast/East Asian and mostly Caucasian subject cohorts, suggesting absence of pharmacokinetic ethnic sensitivity. Therefore, Compound 2 does not require dose adjustment.

### Urine Excretion

Following single oral administration of Compound 2 at 250 to 1500 mg, urine elimination was low for Compound 1 and Compound 3 and was modest for Compound 7 and Compound 6 (Table 9). Total urine recovery ranged from approximately 20% to approximately 30% across doses. Renal clearance of the nucleoside metabolites Compound 6 and Compound 7 exceeded estimated glomerular filtration rate suggesting involvement of active secretion in their renal environment.

### Table 14. Urine Elimination of Compounds Following Single Oral Administration of Compound 2

Compound Name Urine Elimination
   Compound 1 <3%
   Compound 3 <1%
   Compound 6 6.7-8.8%
   Compound 7 10.7-17.9%

### Example 25. Pharmacokinetic Study in MAD Cohorts

Healthy males and females 18-65 years of age were enrolled in multiple ascending dose (MAD) and randomized to receive oral Compound 2 or placebo according to Tables 4 and 7.

Plasma and urine samples were collected at pre-determined timepoints and quantitated for Compound 1 and its metabolites Compounds 3, 6, and 7 using LC-MS/MS. Pharmacokinetic analyses were performed using non-compartmental approaches.

Plasma exposure of Compound 6 increased by approximately 25, 60 and 80% with QD, BID, and TID respectively due to its long plasma half-life. This is reflective of rapid accumulation and sustained intracellular exposure of the active metabolite Compound 5.

The 750 mg TID dose led to a rapid increase in plasma concentration of Compound 6, exceeding the 90% effective concentration (EC₉₀) of the drug in inhibiting dengue virus replication in vitro (0.64 μM). These levels were maintained over the treatment period (FIG. 11).

TABLE 15
Summary of steady-state plasma pharmacokinetic
parameters of Compound 1

| Compound 1 | | Cₘₐₓ (ng/mL) | Tₘₐₓ (h) | AUCₜₐᵤ (ng/mL*h) |
|---|---|---|---|---|
| 1000 | mg QD | 3810 ± 1670 | 0.5 (0.5-0.5) | 3090 ± 1530 |
| 750 | mg BID | 3490 ± 1280 | 0.5 (0.5-0.8) | 2609 ± 640 |
| 750 | mg TID | 3010 ± 754 | 0.5 (0.5-1.0) | 2540 ± 572 |

TABLE 16
Summary of steady-state plasma pharmacokinetic
parameters of Compound 3

| Compound 1 | | Cₘₐₓ (ng/mL) | Tₘₐₓ (h) | AUCₜₐᵤ (ng/mL*h) |
|---|---|---|---|---|
| 1000 | mg QD | 392 ± 143 | 1.0 (0.8-2.0) | 1650 ± 498 |
| 750 | mg BID | 365 ± 159 | 1.0 (0.8-2.0) | 1330 ± 503 |
| 750 | mg TID | 245 ± 30 | 1.5 (0.8-2.0) | 944 ± 173 |

TABLE 17
Summary of steady-state plasma pharmacokinetic
parameters of Compound 6

| Compound 1 | | Cₘₐₓ (ng/mL) | Tₘₐₓ (h) | AUCₜₐᵤ (ng/mL*h) |
|---|---|---|---|---|
| 1000 | mg QD | 241 ± 56 | 4.0 (3.0-4.0) | 3120 ± 694 |
| 750 | mg BID | 288 ± 58 | 3.5 (3.0-4.0) | 2680 ± 730 |
| 750 | mg TID | 330 ± 98 | 3.5 (3.0-6.0) | 2370 ± 743 |

TABLE 18
Summary of steady-state plasma pharmacokinetic
parameters of Compound 7

| Compound 1 | | Cₘₐₓ (ng/mL) | Tₘₐₓ (h) | AUCₜₐᵤ (ng/mL*h) |
|---|---|---|---|---|
| 1000 | mg QD | 1200 ± 284 | 0.9 (0.8-2.0) | 8290 ± 2290 |
| 750 | mg BID | 1470 ± 404 | 1.5 (0.8-2.2) | 7960 ± 2970 |
| 750 | mg TID | 1600 ± 640 | 1.5 (1.0-2.0) | 22300 ± 14100 |

In tables 15-18, Cₘₐₓ and AUCₜₐᵤ are represented by the mean±standard deviation. Tₘₐₓ is represented as the median (minimum-maximum). AUCₜau indicates the area under plasma concentration-time curve over the dosing interval.

### Example 26. Antiviral Assays

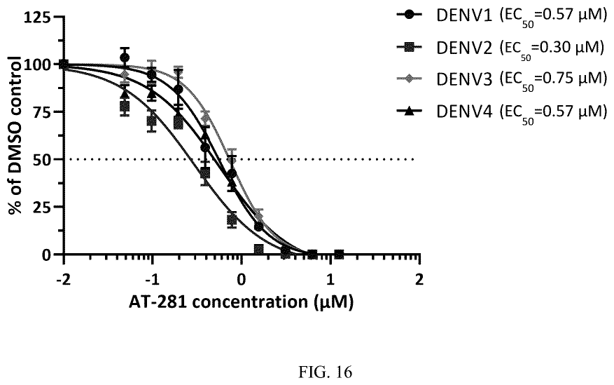

Transcripts of the dengue viruses (DENV1 WP, DENV2 NGC, DENV3 VN32, DENV4 MY01) were modified with a nanoluciferase reporter between the 5′UTR and capsid gene as previously described (Baker et al., 2020b, 2020a) to create tagged viruses that are stable with cell culture passaging while producing a robust luciferase signal after inoculation for efficient screening of antiviral activity. Eight 2-fold serial dilutions of AT-281 were mixed with each reporter virus (MOIs of 0.1 for DENV-1, DENV-2 and DENV-3; MOI of 0.001 for DENV-4) and added to plates containing Huh-7 cells (RRID: CVCL 0336) that were seeded at 1×104 cells per well in a 96-well plate the previous day in Dulbecco's Modified Eagle Medium (Invitrogen, Carlsbad, CA) with 2% fetal bovine serum (Hyclone, Logan, UT). Cells were washed 48 h post infection three times with phosphate-buffered saline, followed by addition of NanoGlo® substrate diluted 1:100 in NanoGlo® Assay Buffer (Promega, Madison, WI). Luciferase activities were read by a BioTek Cytation 5 plate reader after 3 minutes. Data were analyzed with GraphPad Prism 9 software. Luciferase activities were normalized to DMSO treatment samples which were set to 100%. Error bars represent mean±SD. Results are representative of three independent experiments with each one analyzed in triplicate. The antiviral activity against the viruses tested can be found in FIG. 16.

### Example 27. Expression of DENV NS5, RdRp and MTase Domains

The proteins used in the thermal shift assay (Example 31) were prepared as described herein. Full length DENV NS5 and RdRp domains used in this study were expressed under the control of a T7-promoter in pET28a vectors in Escherichia coli (E. coli) NEB C₂₅₆₆ cells (New England Biolabs) carrying the pRARE2LacI (Novagen) plasmid. Proteins were expressed overnight at 17° C. in TB (with 25 μg/mL Kanamycin and 17 μg/mL Chloramphenicol), following induction with 100 μM IPTG and 2% EtOH (% v/v) at an OD600 of 0.8-1.

DENV MTases were expressed under the control of a T7-promoter in pDEST17 vectors in Escherichia coli (E. coli)) NEB C2566 cells (New England Biolabs) carrying the pRARE2LacI (Novagen) plasmid. Proteins were expressed overnight at 17° C. in TB (with 100 μg/mL Ampicillin and 17 μg/mL Chloramphenicol), following induction with 200 μM IPTG and 2% EtOH (% v/v) at an OD600 of 0.8-1.

### Example 28. Purification of DENV NS5

The DENV NS5 used in the thermal shift assay (Example 31) were purified as described herein. The cells were disrupted by sonication on ice in a lysis buffer (50 mM NaP pH 8, 1 M NaCl, 20% glycerol) supplemented with, 1.0 mg/mL Lysozyme, 22 μg/mL DNase, 1.6% igepal, 0.5 mM TCEP and a complete protease inhibitor cocktail (COC) from Roche. Protein from the soluble fraction was loaded onto TALON® Superflow™ cobalt-based IMAC resin (Cytiva), washed 5 times with lysis buffer, 10 times with lysis buffer (igepal free) prior to elution with a lysis buffer supplemented with 250 mM imidazole and 250 mM glycine. Imidazole was removed by an overnight dialysis in a dialysis buffer (50 mM NaP pH 8, 20% glycerol, 150 mM NaCl, 250 mM glycine and 0.5 mM TCEP) before to perform a last step purification using a size exclusion chromatography (SEC) HiLoad® 16/600 Superdex® 200 μg column in a buffer (10 mM HEPES pH 8, 300 mM NaCl, 10% glycerol and 0.5 mM TCEP). Protein was then concentrated to 10 to 15 mg·mL-1 and stored at −80° C. after a last dialysis in the SEC buffer supplemented with 40% of glycerol.

### Example 29. Purification of DENV RdRp Domains

The DENV RdRp used in the thermal shift assay (Example 31) were purified as described herein. The cells were disrupted by sonication on ice in a lysis buffer (50 mM NaP pH 8, 1 M NaCl, 20% glycerol) supplemented with, 1.0 mg/mL Lysozyme, 22 μg/mL DNase, 1.6% igepal, 0.5 mM TCEP and a complete protease inhibitor cocktail (COC) from Roche. Protein from the soluble fraction were loaded onto TALON® Superflow™ cobalt-based IMAC resin (Cytiva), washed 5 times with lysis buffer, 10 times with lysis buffer (igepal free) prior to elution with a lysis buffer supplemented with 250 mM imidazole and 250 mM glycine. Imidazole was removed by an overnight dialysis in a dialysis buffer (50 mM NaP pH 8, 20% glycerol, 150 mM NaCl, 250 mM glycine and 0.5 mM TCEP) before to perform a last step purification using a GE Hi-trap Heparine column. Protein was then concentrated to 10 to 15 mg·mL-1 and stored at −80° C. after a last dialysis in a final buffer containing 20 mM HEPES pH 8, 300 mM NaCl, 40% of glycerol and 0.5 mM TCEP.

### Example 30. Purification of DENV MTase Domains

The DENV MTase used in the thermal shift assay (Example 31) were purified as described herein. The cells were disrupted by sonication on ice in a lysis buffer (50 mM HEPES pH 7.5, 10% glycerol, 500 mM NaCl and 5 mM Imidazole) supplemented with 0.2 mM Benzamidine, 1 mg/mL Lysozyme, 22 μg/mL DNase and 0.5 mM TCEP. Protein from the soluble fraction were loaded onto TALON® Superflow™ cobalt-based IMAC resin (Cytiva) and washed with lysis buffer (1M NaCl) prior to elution with 250 mM imidazole. The protein was finally purified with a SEC (HiLoad® 16/600 Superdex® 200 μg) in a buffer of 50 mM HEPES pH 8, 300 mM NaCl and 0.5 mM TCEP. Protein was then concentrated to 10 to 15 mg·mL-1 and stored at −80° C. after a last dialysis in a final buffer containing 20 mM HEPES pH 8, 300 mM NaCl, 40% of glycerol and 0.5 mM TCEP.

### Example 31. Thermal Shift Assay

The thermal stability of the flavivirus proteins and their interaction with compounds were evaluated by using a thermal shift assay. Reactions were performed in white frame star PCR 96-well plates (ref 044705) with a final volume of 20 μL. Regarding the DENV NS5, Polymerase and MTase, the best conditions were found by using a buffer containing 20 mM Hepes pH7.5, 50 mM NaCl, 5 mM DTT, 1000 Glycerol and 1M Sorbitol. 0.4 μL of GTP, Sinefungin or AT-9010 were disposed in the plate (100 μM final concentration). Water was used as a control without compound. 2 μM of NS5, 2 μM of Polymerase or 6 μM of MTase were mixed in the buffer with 2.5×, 0.75× or 1.5× of dye respectively (final concentrations). The mix of protein and Protein thermal shift kit from ThermoFisher (ref 4461146) was prepared and 19.6 μl were dispensed into each well.

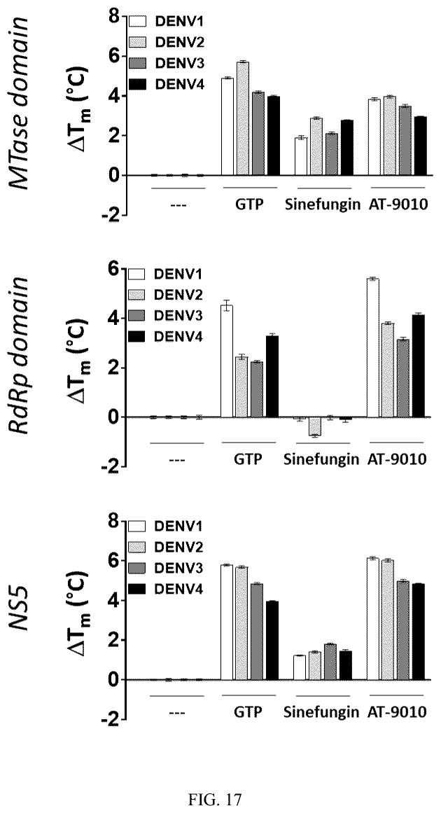

The plate was sealed with adhesive film and then submitted to a run on a CFX96 RT-PCR (Bio-Rad). The program started by a 1 min equilibration phase at 20° C., followed by a temperature gradient of 0.5° C./30 s, from 20° C. to 95° C., recording fluorescence every 0.5° C. Fluorescence was recorded using the FRET channel. Every experiment was performed in triplicate. Curves were plotted and analyzed to determine the melting temperature by using the Boltzmann sigmoidal equation with the GraphPad Prism software. Graphical representation of the data in the table below is shown in FIG. 17.

TABLE 19
Data from thermal shift assay

| | ΔT_{M}(° C.) | DENV1 | DENV2 | DENV3 | DENV4 |
|---|---|---|---|---|---|
| MTase | GTP | 4.9 ± 0.1 | 5.7 ± 0.1 | 4.1 ± 0.1 | 4.0 ± 0.1 |
| Domain | Sinefungin | 1.9 ± 0.1 | 2.9 ± 0.1 | 2.1 ± 0.1 | 2.8 ± 0.1 |
| | AT-9010 | 3.8 ± 0.1 | 4.0 ± 0.1 | 3.5 ± 0.1 | 2.9 ± 0.1 |
| RdRp | GTP | 4.5 ± 0.2 | 2.4 ± 0.1 | 2.2 ± 0.1 | 3.3 ± 0.1 |
| Domain | Sinefungin | −0.1 ± 0.1 | −0.8 ± 0.1 | 0.0 ± 0.1 | −0.1 ± 0.1 |
| | AT-9010 | 5.6 ± 0.1 | 3.8 ± 0.1 | 3.2 ± 0.1 | 4.1 ± 0.1 |
| NS5 | GTP | 5.8 ± 0.1 | 5.7 ± 0.1 | 4.8 ± 0.1 | 3.9 ± 0.1 |
| | Sinefungin | 1.2 ± 0.1 | 1.4 ± 0.1 | 1.8 ± 0.1 | 1.4 ± 0.1 |
| | AT-9010 | 6.1 ± 0.1 | 6.0 ± 0.1 | 5.0 ± 0.1 | 4.8 ± 0.1 |

### Example 32. pppApG Primer Synthesis

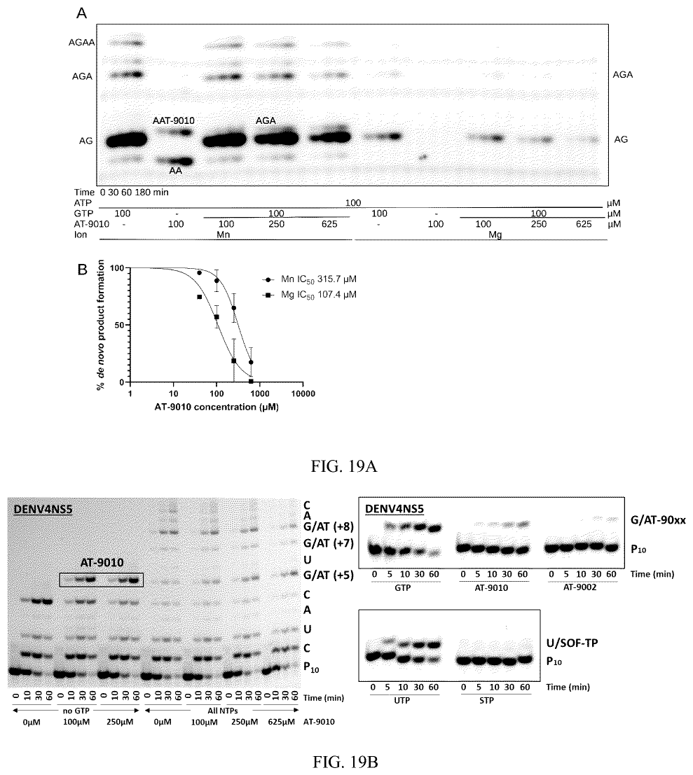

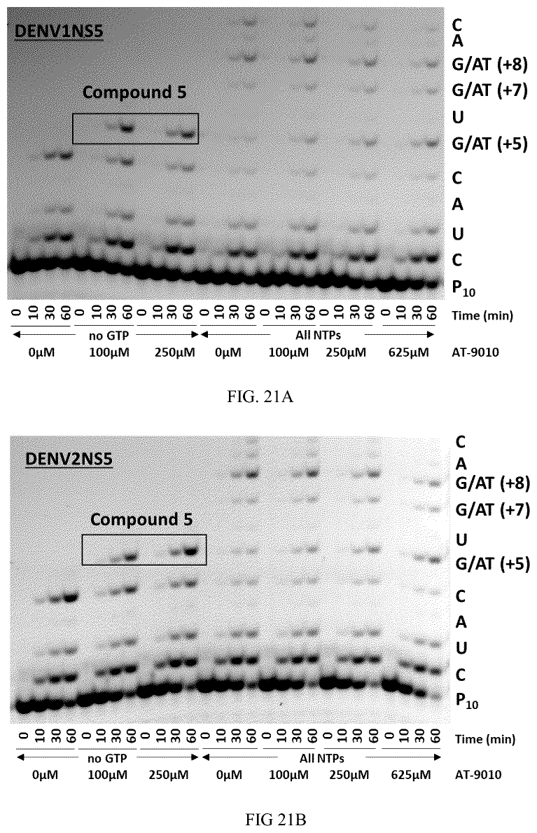

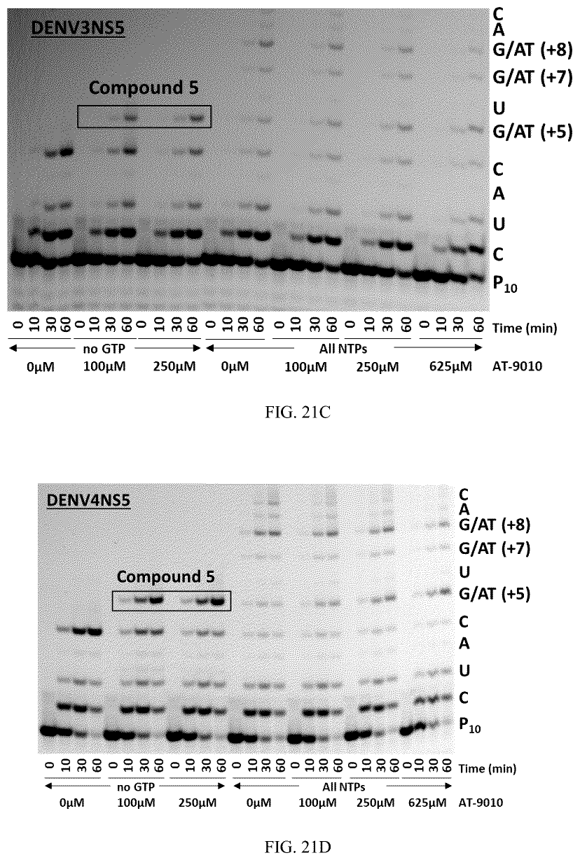

Reactions were carried out in the presence of 2 mM MnCl₂ or 5 mM MgCl₂, the optimum concentrations of these catalytic ions for RNA synthesis by DENV2 RdRp (Selisko et al., 2006). Discrimination of AT-9010 against GTP was measured in four independent reactions, with similar results as those shown in FIGS. 19B and 21A, 21B, 21C, and 21D.

### Example 33. MTase Activity Assay

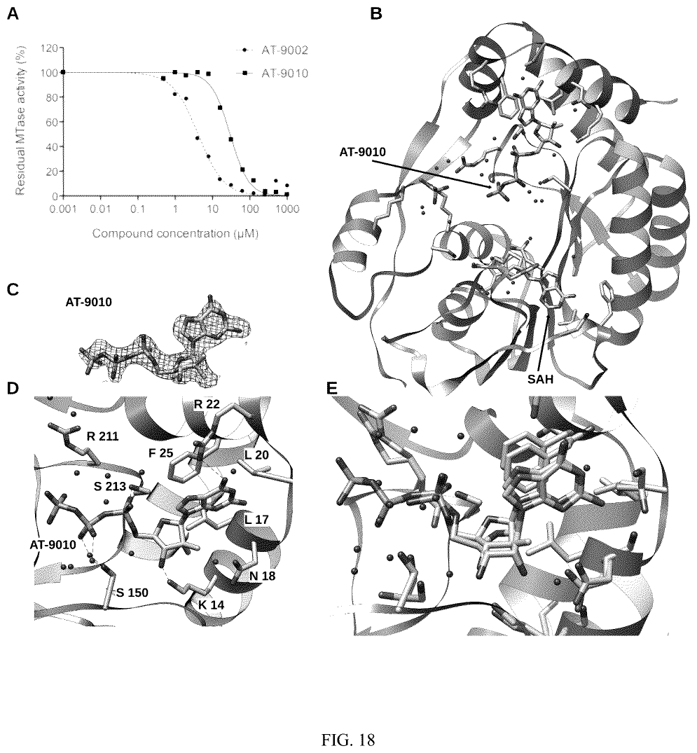

The transfer of tritiated methyl from [3H]-SAM onto RNA substrate was monitored by filter-binding assay (FBA), performed according to the method described previously (Paesen et al., 2015). The recombinant 2′-O-methyltransferase domain of the non-structural protein NS5 from DENV serotype 2 (DENV NS5 MTase) corresponding to residues 1-296 of NS5 was expressed and purified as described previously (Egloff et al., 2002). FBA was carried out in reaction mixture [40 mM Tris-HCl (pH 8.0), 1 mM DTT, 2 μM SAM and 0.1 μM 3H-SAM (Perkin Elmer)] in the presence of 0.7 μM mGpppAC4 synthetic RNA and DENV2 NS5MTase protein (500 nM). The enzyme was first mixed with the compound (0.5 to 1000 μM) suspended in water before the addition of RNA substrate and SAM and then incubated at 30° C. Reaction mixtures were stopped after 30 min by their 10-fold dilution in ice-cold water. Samples were transferred to diethylaminoethyl (DEAE) filtermat (Perkin Elmer) using a Filtermat Harvester (Packard Instruments). The RNA-retaining mats were washed twice with 10 mM ammonium formate pH 8.0, twice with water and once with ethanol. They were soaked with scintillation fluid (Perkin Elmer), and 3H-methyl transfer to the RNA substrates was determined using a Wallac MicroBeta TriLux Liquid Scintillation Counter (Perkin Elmer). For IC₅₀ measurements, values were normalized and fitted with Prism (GraphPad software) using the following equation: Y=100/(1+((X/IC₅₀)\hat{ }Hillslope)). IC₅₀ is defined as the inhibitory compound concentration that causes 50% reduction in enzyme activity. Data curves can be found in FIG. 18.

### Example 34. Incorporation of Nucleotide Analogues into RNA

DENV NS5 was pre-incubated with the annealed P10/T20 RNA in an assembly buffer containing 20 mM HEPES pH 7.5, 10% glycerol, 5 mM MgCl₂, and 5 mM DTT for 10 min at 30° C. to create an active RNA elongation complex.

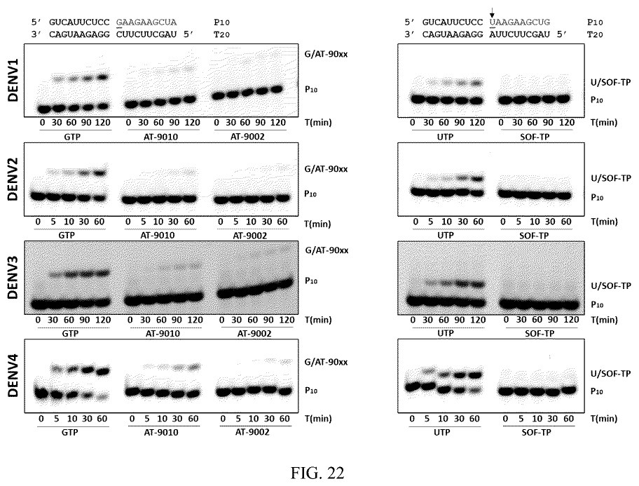

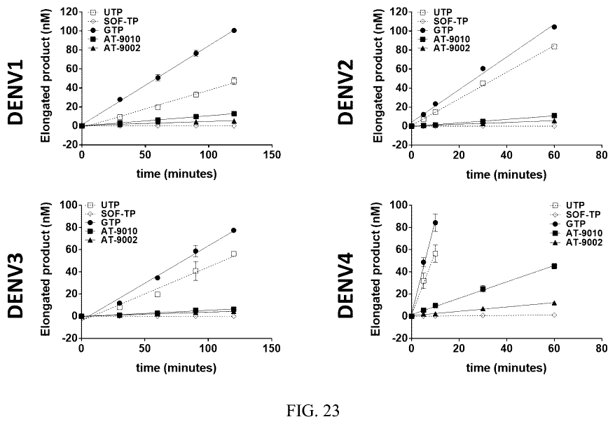

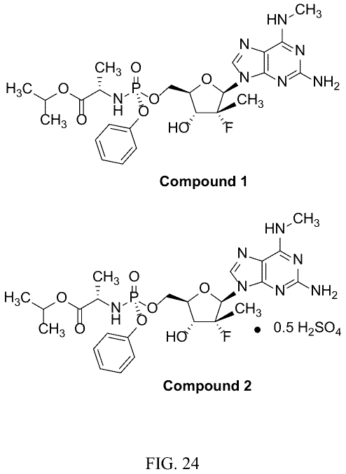

Multiple nucleotide incorporation was tested as follows and the results shown in FIGS. 19A, 21, 22, and 23. Reactions were started by adding AT-9010 with either all four NTPs or UTP, ATP, and CTP only. Final concentrations were 1 μM NS5, 0.25 μM P10/T20, 100 μM each NTP, and between 10 and 625 μM AT-9010 in a final buffer containing 20 mM HEPES pH 7.5, 15% glycerol, 5 mM MgCl2, and 5 mM DTT.

Single nucleotide incorporation was tested as follows. Reactions were started by adding the corresponding NTP or analogue. Final concentrations were 1 μM NS5, 0.25 μM P10/T20, 100 μM NTP or analogue in a final buffer containing 20 mM HEPES pH 7.5, 15% glycerol, 5 mM MgCl2, and 5 mM DTT.

Reactions were quenched at the designated time points in 3× volume FBD stop solution (formamide, 10 mM EDTA) and analyzed on 20% acrylamide-bisacrylamide (19:1) 7 μM urea sequencing gels. RNA products were visualized using a Typhoon FluorImager then analyzed and quantified using ImageQuant software.

### Example 35. Crystallographic and Modeling Studies

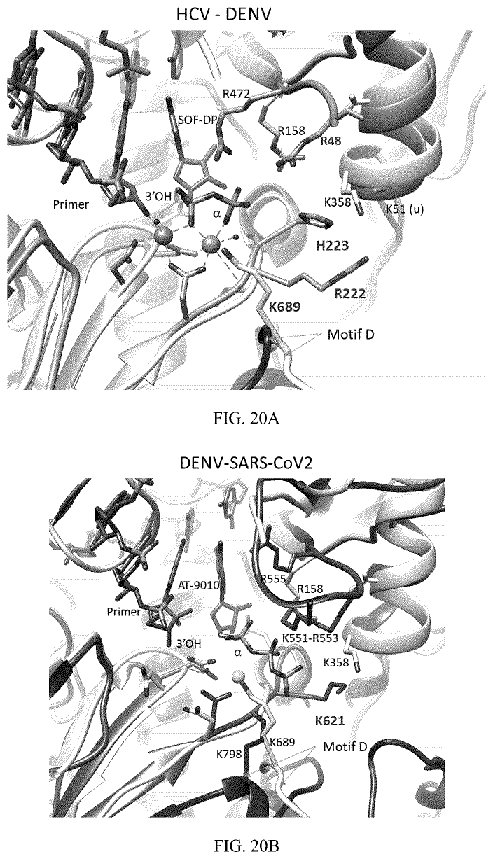

Crystallographic images produced by the protocol described herein are presented in FIGS. 20A and 20B. The construct used for crystallization was the same as described in (Barral et al., 2013) with a cleavable Hexahistidine-Thioredoxin tag at the N-terminus of DENV3 NS5 coding sequence (amino acids region 1-277) in plasmid pMcox20A. Transformed E. coli T7 express cells (New England BioLabs) were grown at 37° C. in LB medium supplemented with 100 μg/mL ampicillin, to an OD600 nm of 0.6-0.8. Expression of the recombinant protein was induced by the addition of 100 μM IPTG, and incubation was continued for a further 16 h at 17° C. The cells were harvested by centrifugation at 8000 g for 10 min at 4° C. and stored at −80° C. The purification of the protein and the tag removal was performed in non-denaturing conditions as described in (Lantez et al., 2011) by using TALON® Superflow™ cobalt-based IMAC resin (Cytiva) for affinity chromatography. The final size exclusion chromatography step was performed using a Superdex S200 HiLoad 16/60 column with a buffer containing 20 mM HEPES pH 7.5, 200 mM NaCl, 10% glycerol and 2 mM DTT.

Crystallization conditions were adapted from (Lim et al., 2011) using the sitting-drop vapor diffusion method using crystallization buffer. All crystals were grown at 293.15 K, using a 1:2 ratio of protein (10 mg/mL) to precipitant solution (22% PEG 8000, 200 mM NaCl, 20 mM Tri-Sodium Citrate, 100 mM Tris pH 8.5). Crystals grew in 4 days and were soaked overnight with 1 mM of AT-9010 (final concentration). All crystals were cryo-protected with reservoir solution supplemented with 10% glycerol, and flash-frozen in liquid nitrogen at 100 K.

Data collection and diffraction data were collected at Soleil synchrotron. Original native data set was collected on Proxima2. Data set was processed and analysed with autoPROC toolbox (Vonrhein et al., 2011). Structure was solved by molecular replacement using PHASER (McCoy et al., 2007) and PDB 4CTK as a reference model. As previously observed the crystal Asymmetric Unit is composed of 2 chains, however the additional positive density blobs found in both chain is not equivalent. In one chain the extra density corresponds to a single molecule of SAM while in the other chain the positive density corresponds to one SAH and an AT-9010 molecules. The model was build using COOT (Emsley and Cowtan, 2004) and refined at 1.90 Å using PHENIX (Adams et al., 2010). The structure was checked and confirmed to have good stereochemistry according to MOLPROBITY (Chen et al., 2010). Data collection and refinement statistics are listed in Table 21. Structural analysis and figures was done using UCSF CHIMERA (Pettersen et al., 2004).

TABLE 20
Conservation and identification of essential amino acids
in contact with the phosphates of the incoming nucleotide
in viral RdRps HCV NS5b, DENV2 NS5, and SARS-CoV2 nsp12.

| Virus | | | |
|---|---|---|---|
| Contacts at | HCV | DENV | SARS-CoV-2 |
| α- phosphate | R158 | R472 | R555 |
| β- phosphate | R48 | — | R553* |
| γ- phosphate | K51, R22, H223 | K358 | K551* |
| Motif D residue | — | K689 | K798 |
| Other features | H223 corresponds | — | K551 and R553 |
| | to SARS Motif | | CoV unique Motif |
| | D K621 | | F K621 corresponds |
| | | | to HCV H223 |

TABLE 21
Crystallographic parameters

| Structure solution & refinement | | |
|---|---|---|
| No. of reflections, working set | 45347 | (4551) |
| No. of reflections, test set (%) | 2195 (217), 5.0% | |
| R_{work} | 0.20 | (0.27) |
| R_{free} | 0.24 | (0.33) |

| | |
|---|---|
| No. of non-H atoms | 4589 |
| Protein | 4162 |
| Ligand | 65 |
| Water | 362 |
| R.M.S. deviations | Protein residues 516 |
| Bonds (Å) | 0.013 |
| Angles (°) | 1.21 |
| Average B-factors (Å²) | 29.38 |
| Protein | 28.68 |
| Ligand | 41.71 |
| Water | 35.22 |
| Ramachandran Plot | |
| Favoured (%) | 98.04 |
| Allowed (%) | 1.96 |
| Outliers (%) | 0.00 |
| Clashcore | 5.21 |
| PDB code | 8BCR |

| | DENV3 MTase complexed | |
|---|---|---|
| Data processing | with soaked AT-9010 | |
| Wavelength (Å) | 0.9801 | |
| Space group | P 21 21 2 | |
| a, b, c (Å) | 61.26, 184.13, 52.15 | |
| α, β, γ (°) | 90.00, 90.00, 90.00 | |
| Resolution range (Å) | 45.38-1.9 | (1.97-1.9) |
| Total No. of reflections | 583677 | (27381) |
| No. of unique reflections | 45354 | (2284) |
| Completeness (%) | 96.0 | (97.8) |
| Multiplicity | 12.9 | (12.0) |
| I/σ(I) | 18.5 | (4.7) |
| R meas | 0.093 | (0.46) |
| R merge | 0.089 | (0.44) |
| R-pim | 0.026 | (0.13) |
| CC½ | 0.99 | (0.94) |

| | |
|---|---|
| Wilson B-factor (Å) | 24.6 |

### Example 36. Synthesis of Compound 1

Compound 1 (the free base form of Compound 2) can be synthesized, for example, by reacting a compound of the formula Intermediate A with Intermediate B and an activator to form a compound of the formula Intermediate C, which is then reacted with phenol to form Compound 1:

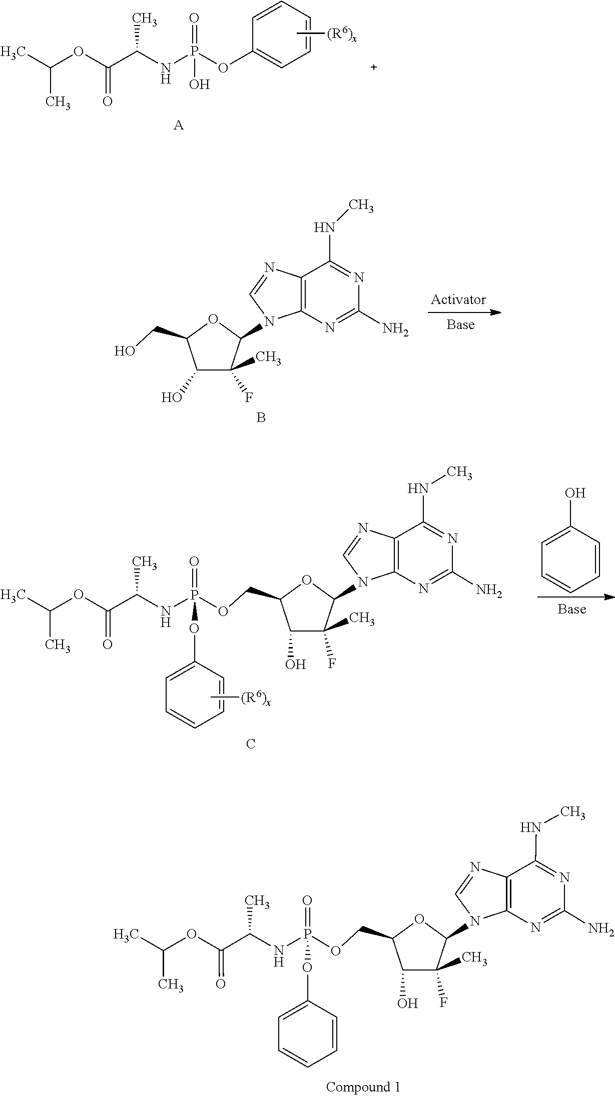

wherein R⁶ is independently selected at each instance from fluoro, chloro, bromo, cyano, haloalkyl such as trifluoromethyl, difluoromethyl, fluoromethyl, azido, and nitro; and
x is 1, 2, or 3.

Reaction of a compound of the formula Intermediate A, Intermediate B, an activator and a trialkylamine base preferentially forms the S_{P} phosphoramidate (as described in WO 2022/040473A1 assigned to Atea Pharmaceuticals).

As used herein, an “activator” is a uronium-based peptide coupling reagent, including but not limited to 1-[Bis(dimethylamino)methylene]-1H-1,2,3-triazolo[4,5-b]pyridinium 3-oxide hexafluorophosphate (HATU), 2-(1H-benzotriazol-1-yl)-1,1,3,3-tetramethyluronium hexafluorophosphate (HBTU), 2-(1H-Benzotriazole-1-yl)-1,1,3,3-tetramethylaminium tetrafluoroborate (TBTU) and 1-Cyano-2-ethoxy-2-oxoethylidenaminooxy)dimethylamino-morpholino-carbenium hexafluorophosphate (COMU).

As used herein, a “trialkylamine base” is an amine base with three C₁₋₆ alkyl groups, including but not limited to triethylamine or diisopropylethylamine.

As used herein, a “pyridine base” is an optionally substituted pyridine molecule, including but not limited to pyridine, 2,6-dimethylpyridine (lutidine), or 2,4,6-trimethylpyridine (collidine).

If the S_{P} phosphoramidate has electron withdrawing substituents on the aryl ester (Intermediate C), then the S_{P} compound can be reacted further. The phosphorus stereochemistry can be inverted by reaction of the S_{P} phosphoramidate (Intermediate C) with phenol and a base to form the R_{P} phosphoramidate (Compound 1).

### Synthesis of Intermediate A

Intermediate A can be synthesized through a multistep displacement of phosphorus chloride (Intermediate E) followed by reductive deprotection of the benzyl protecting group.

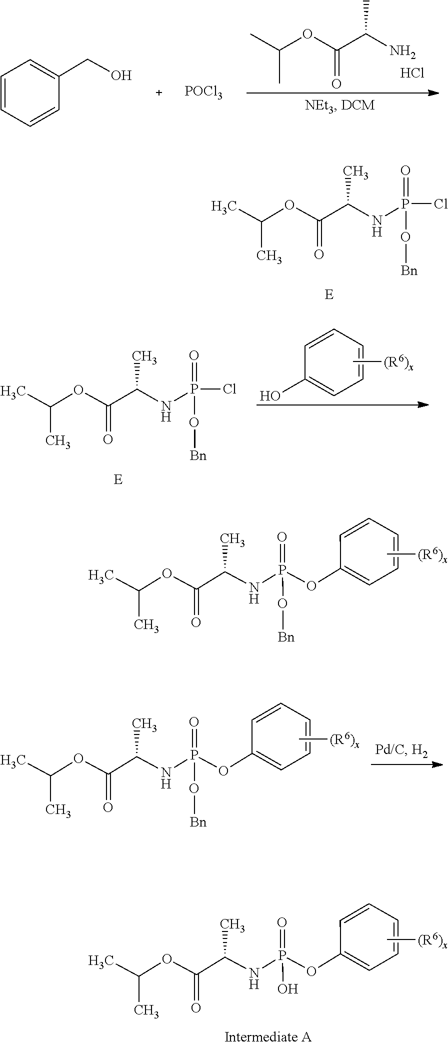

In certain embodiments, the electron withdrawing group on the phenol of Intermediate A is an ortho-fluoro substituent (Intermediate A-1):

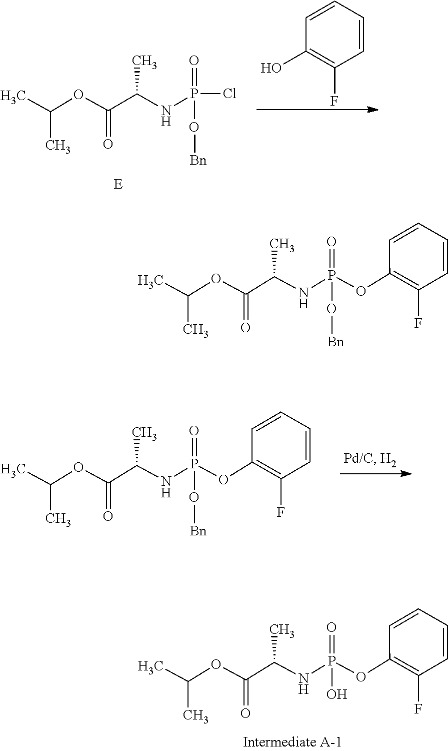 [SMILES: CC(C)OC(=O)[C@H](C)NP(=O)(Cl)OCc1ccccc1 ; Oc1ccccc1F ; CC(C)OC(=O)[C@H](C)NP(=O)(OCc1ccccc1)Oc1ccccc1F ; CC(C)OC(=O)[C@H](C)NP(=O)(OCc1ccccc1)Oc1ccccc1F ; CC(C)OC(=O)[C@H](C)NP(=O)(O)Oc1ccccc1F]

In certain embodiments, Intermediate A has one or more electron withdrawing substituents on the phenol. Electron withdrawing substituents include but are not limited to fluorine, chlorine, cyano, azido, nitro, trifluoromethyl, difluoromethyl, fluoromethyl, ester and amide. In certain embodiments, Intermediate A is used in the form of a salt. In certain embodiments, Intermediate A is used as the dihydroquinine salt.

### Example 37. Phenol Stereoinversion Reaction

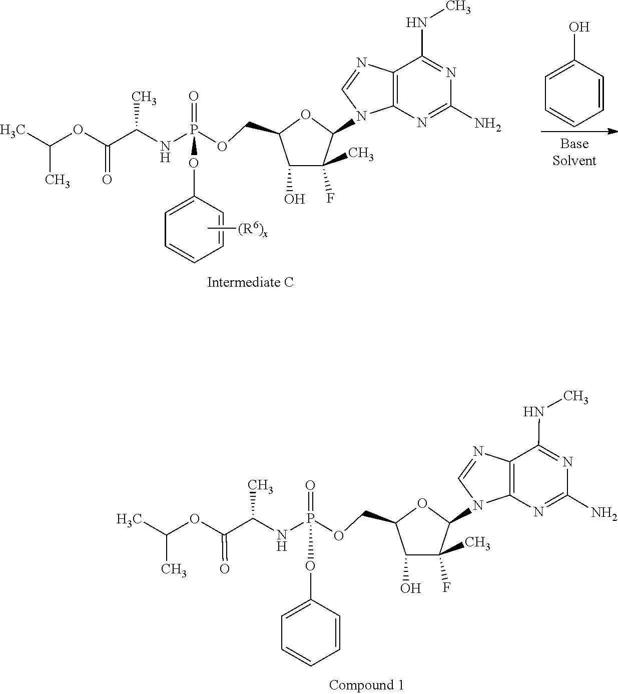

### Amine Bases

The reaction of Intermediate C with phenol to form Compound 1 can be performed for example using a base such as an amine base, for example a guanidine base. Examples of guanidine bases are Bases A, B, C, D, E, F, G, and J. Bases A, C, D and J provided the highest yields of Compound 1. Intermediate C-1 was used as a model substrate for the reaction.

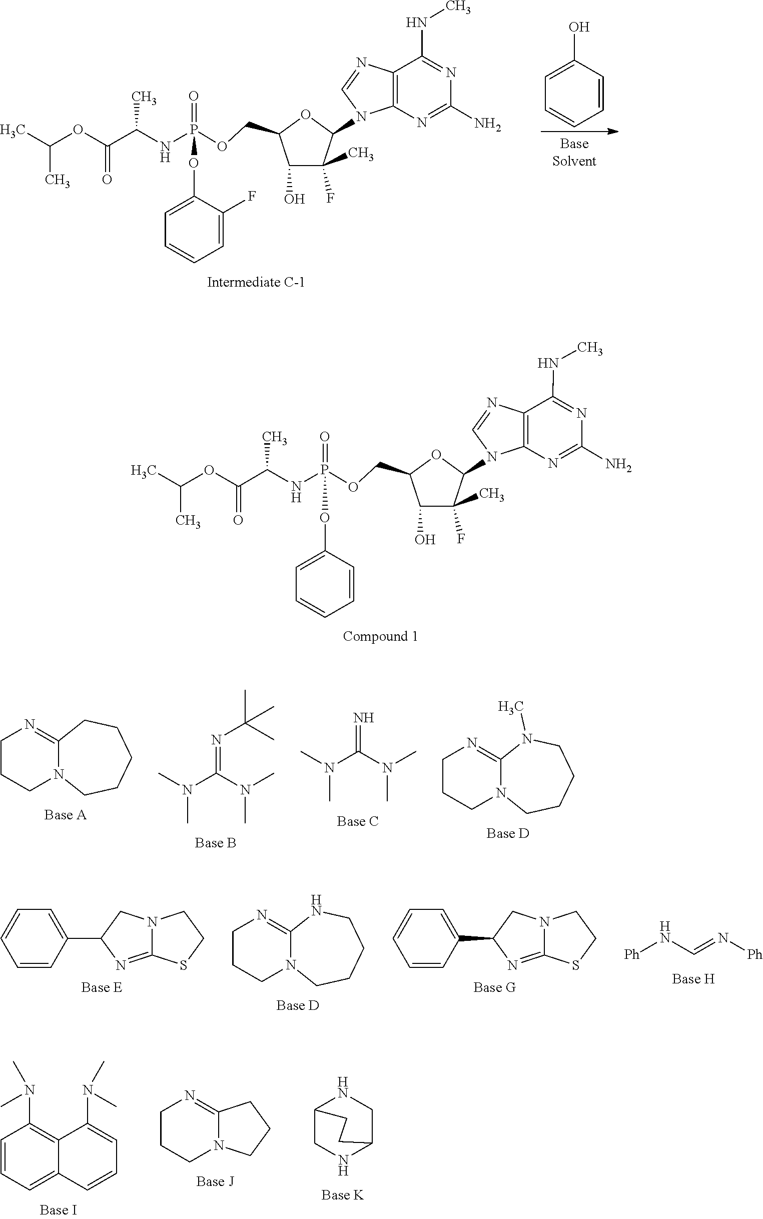

TABLE 22
Base Analysis

| | | | | | Compound 1 |
|---|---|---|---|---|---|
| Intermediate | | | | Temp. | HPLC Area |
| C-1 | Phenol | Base | Solvent | (° C.)/Time | Percent |
| 0.3 g | 0.28 g | A | DMSO | 20-30° C. | 53.31 |
| 1.0 eq. | 6.0 eq. | 0.46 g 6.0 eq | 6 mL | 8 h | |
| 0.3 g | 0.28 g | A | DMSO | 20-30° C. | 45.53 |
| 1.0 eq. | 6.0 eq. | 0.46 g 6.0 eq | 6 mL | 21 h | |
| 0.3 g | 0.28 g | B | DMSO | 20-30° C. | 32.98 |
| 1.0 eq. | 6.0 eq. | 0.51 g 6.0 eq | 6 mL | 8 h | |
| 0.3 g | 0.28 g | B | DMSO | 20-30° C. | 16.95 |
| 1.0 eq. | 6.0 eq. | 0.51 g 6.0 eq | 6 mL | 21 h | |
| 0.3 g | 0.28 g | C | DMSO | 20-30° C. | 70.06 |
| 1.0 eq. | 6.0 eq. | 0.35 g 6.0 eq | 6 mL | 8 h | |
| 0.3 g | 0.28 g | C | DMSO | 20-30° C. | 65.53 |
| 1.0 eq. | 6.0 eq. | 0.35 g 6.0 eq | 6 mL | 21 h | |
| 0.3 g | 0.28 g | D | DMSO | 20-30° C. | 43.22 |
| 1.0 eq. | 6.0 eq. | 0.46 g 6.0 eq | 6 mL | 8 h | |
| 0.3 g | 0.28 g | D | DMSO | 20-30° C. | 28.66 |
| 1.0 eq. | 6.0 eq. | 0.46 g 6.0 eq | 6 mL | 21 h | |
| 0.3 g | 0.28 g | E | DMSO | 20-30° C. | N.D. |
| 1.0 eq. | 6.0 eq. | 0.72 g 6.0 eq | 6 mL | 8 h | |
| 0.3 g | 0.28 g | E | DMSO | 20-30° C. | N.D. |
| 1.0 eq. | 6.0 eq. | 0.72 g 6.0 eq | 6 mL | 21 h | |
| 0.3 g | 0.28 g | F | DMSO | 20-30° C. | 4.07 |
| 1.0 eq. | 6.0 eq. | 0.42 g 6.0 eq | 6 mL | 8 h | |
| 0.3 g | 0.28 g | F | DMSO | 20-30° C. | 3.18 |
| 1.0 eq. | 6.0 eq. | 0.42 g 6.0 eq | 6 mL | 21 h | |
| 0.3 g | 0.28 g | G | DMSO | 20-30° C. | N.D. |
| 1.0 eq. | 6.0 eq. | 0.72 g 6.0 eq | 6 mL | 8 h | |
| 0.3 g | 0.28 g | G | DMSO | 20-30° C. | N.D. |
| 1.0 eq. | 6.0 eq. | 0.72 g 6.0 eq | 6 mL | 21 h | |
| 0.3 g | 0.28 g | H | DMSO | 20-30° C. | N.D. |
| 1.0 eq. | 6.0 eq. | 0.59 g 6.0 eq | 6 mL | 8 h | |
| 0.3 g | 0.28 g | H | DMSO | 20-30° C. | N.D. |
| 1.0 eq. | 6.0 eq. | 0.59 g 6.0 eq | 6 mL | 21 h | |
| 0.3 g | 0.28 g | I | DMSO | 20-30° C. | N.D. |
| 1.0 eq. | 6.0 eq. | 0.64 g 6.0 eq | 6 mL | 8 h | |
| 0.3 g | 0.28 g | I | DMSO | 20-30° C. | 0.56 |
| 1.0 eq. | 6.0 eq. | 0.64 g 6.0 eq | 6 mL | 21 h | |
| 1.0 g | 0.94 g | A | DMSO | 20-30° C. | 62.1 |
| 1.0 eq. | 6.0 eq. | 1.52 g 6.0 eq | 20 mL | 8 h | |
| 0.3 g | 0.28 g | J | DMSO | 20-30° C. | 28.57 |
| 1.0 eq. | 6.0 eq. | 0.37 g 6.0 eq | 6 mL | 3 h | |
| 0.3 g | 0.28 g | J | DMSO | 20-30° C. | 63.63 |
| 1.0 eq. | 6.0 eq. | 0.37 g 6.0 eq | 6 mL | 21 h | |
| 0.3 g | 0.28 g | K | THF | 0-10° C. | 0.30 |
| 1.0 eq. | 6.0 eq. | 0.37 g 6.0 eq | 6 mL | 8 h | |
| 0.3 g | 0.28 g | K | THF | 20-30° C. | 2.12 |
| 1.0 eq. | 6.0 eq. | 0.37 g 6.0 eq | 6 mL | 21 h | |

### Activated Phenol Leaving Group

In certain embodiments, Compound 1 is prepared from a S_{P} phosphoramidate Intermediate C, such as Intermediate C-1, C-2, C-3, C-4, C-5, C-6, C-7, and C-8. Intermediate C-1 through C-8 have different electron withdrawing groups on the phenol. These compounds can be reacted with phenol and a base to form Compound 1:

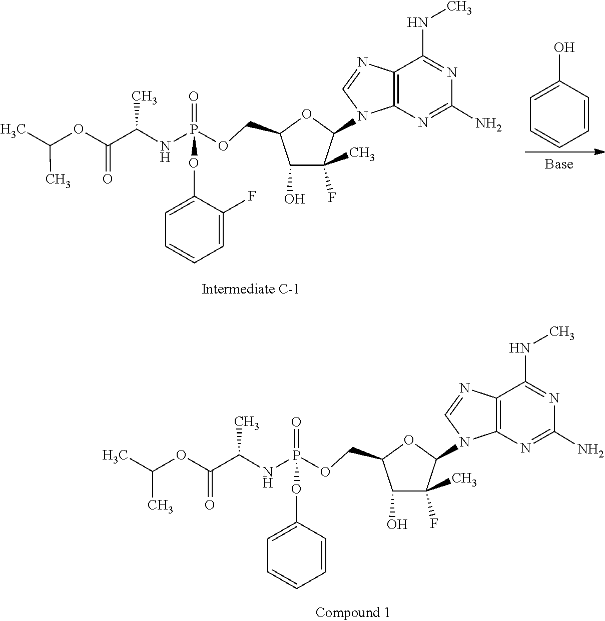 [SMILES: CNc1nc(N)nc2c1ncn2[C@@H]1O[C@H](CO[P@@](=O)(N[C@@H](C)C(=O)OC(C)C)Oc2ccccc2F)[C@@H](O)[C@@]1(C)F ; Oc1ccccc1 ; CNc1nc(N)nc2c1ncn2[C@@H]1O[C@H](CO[P@](=O)(N[C@@H](C)C(=O)OC(C)C)Oc2ccccc2)[C@@H](O)[C@@]1(C)F]

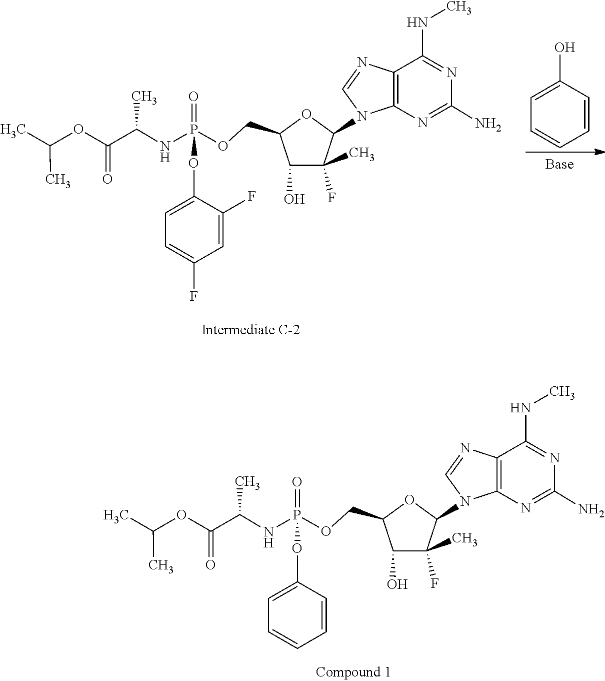 [SMILES: CNc1nc(N)nc2c1ncn2[C@@H]1O[C@H](CO[P@@](=O)(N[C@@H](C)C(=O)OC(C)C)Oc2ccc(F)cc2F)[C@@H](O)[C@@]1(C)F ; Oc1ccccc1 ; CNc1nc(N)nc2c1ncn2[C@@H]1O[C@H](CO[P@](=O)(N[C@@H](C)C(=O)OC(C)C)Oc2ccccc2)[C@@H](O)[C@@]1(C)F]

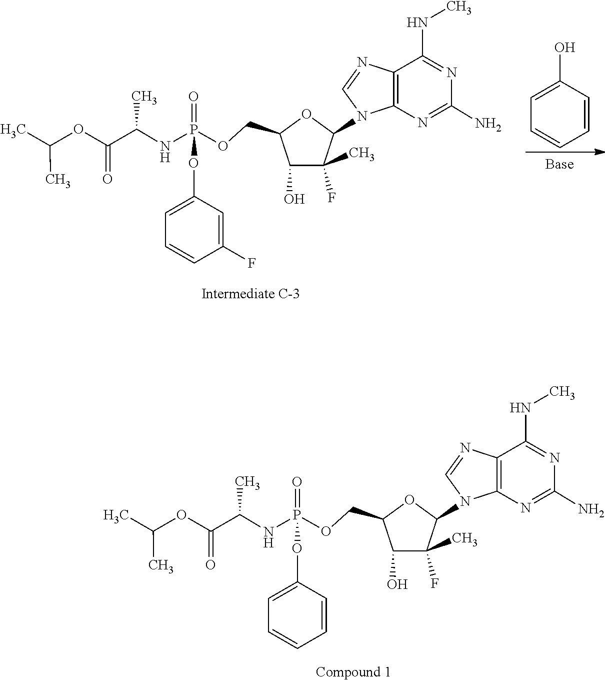 [SMILES: CNc1nc(N)nc2c1ncn2[C@@H]1O[C@H](CO[P@@](=O)(N[C@@H](C)C(=O)OC(C)C)Oc2cccc(F)c2)[C@@H](O)[C@@]1(C)F ; Oc1ccccc1 ; CNc1nc(N)nc2c1ncn2[C@@H]1O[C@H](CO[P@](=O)(N[C@@H](C)C(=O)OC(C)C)Oc2ccccc2)[C@@H](O)[C@@]1(C)F]

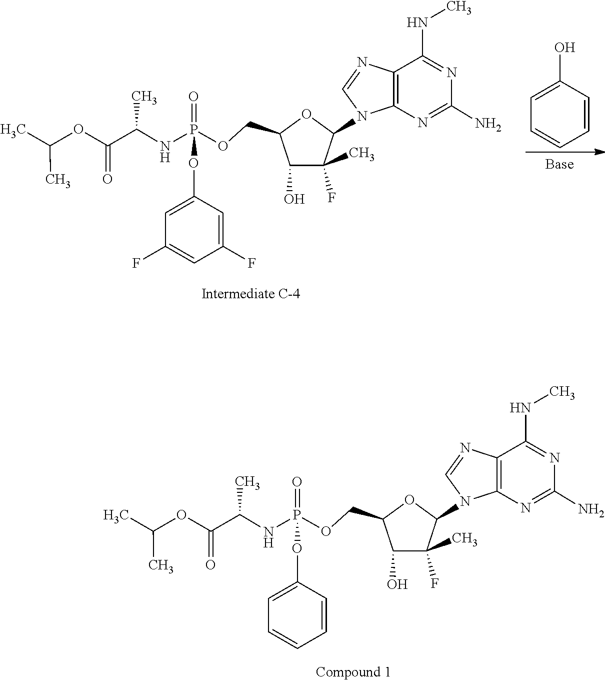 [SMILES: CNc1nc(N)nc2c1ncn2[C@@H]1O[C@H](CO[P@@](=O)(N[C@@H](C)C(=O)OC(C)C)Oc2cc(F)cc(F)c2)[C@@H](O)[C@@]1(C)F ; Oc1ccccc1 ; CNc1nc(N)nc2c1ncn2[C@@H]1O[C@H](CO[P@](=O)(N[C@@H](C)C(=O)OC(C)C)Oc2ccccc2)[C@@H](O)[C@@]1(C)F]

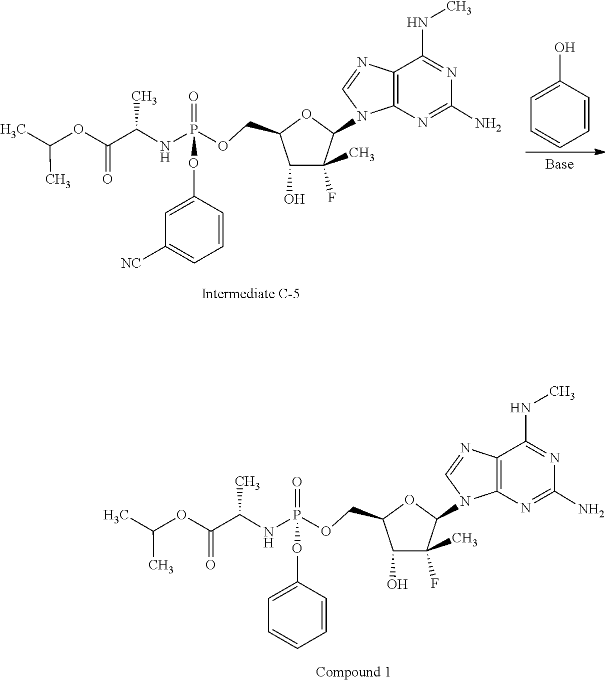 [SMILES: CNc1nc(N)nc2c1ncn2[C@@H]1O[C@H](CO[P@@](=O)(N[C@@H](C)C(=O)OC(C)C)Oc2cccc(C#N)c2)[C@@H](O)[C@@]1(C)F ; Oc1ccccc1 ; CNc1nc(N)nc2c1ncn2[C@@H]1O[C@H](CO[P@](=O)(N[C@@H](C)C(=O)OC(C)C)Oc2ccccc2)[C@@H](O)[C@@]1(C)F]

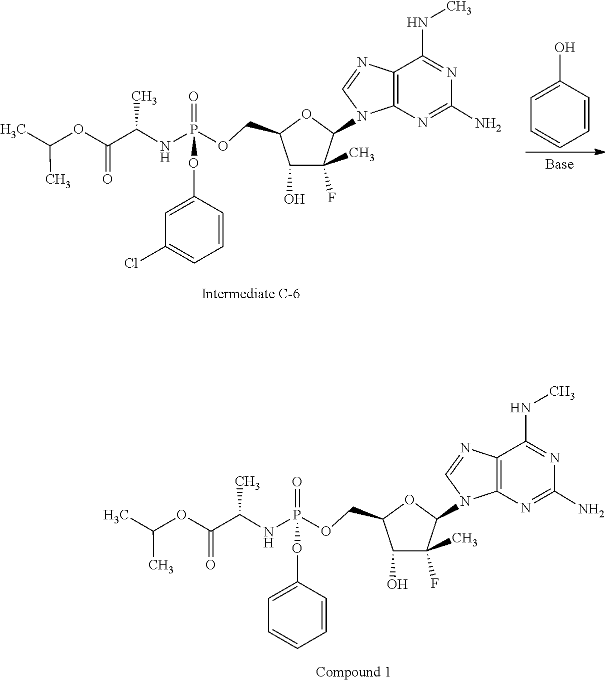 [SMILES: CNc1nc(N)nc2c1ncn2[C@@H]1O[C@H](CO[P@@](=O)(N[C@@H](C)C(=O)OC(C)C)Oc2cccc(Cl)c2)[C@@H](O)[C@@]1(C)F ; Oc1ccccc1 ; CNc1nc(N)nc2c1ncn2[C@@H]1O[C@H](CO[P@](=O)(N[C@@H](C)C(=O)OC(C)C)Oc2ccccc2)[C@@H](O)[C@@]1(C)F]

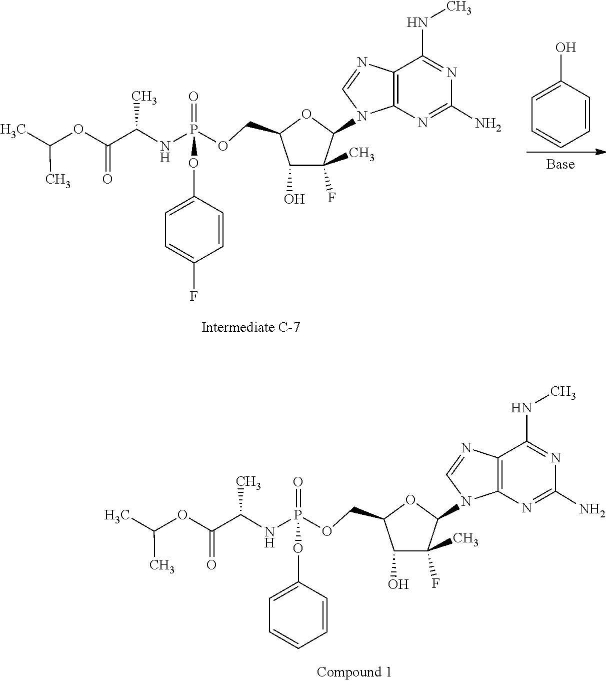 [SMILES: CNc1nc(N)nc2c1ncn2[C@@H]1O[C@H](CO[P@@](=O)(N[C@@H](C)C(=O)OC(C)C)Oc2ccc(F)cc2)[C@@H](O)[C@@]1(C)F ; Oc1ccccc1 ; CNc1nc(N)nc2c1ncn2[C@@H]1O[C@H](CO[P@](=O)(N[C@@H](C)C(=O)OC(C)C)Oc2ccccc2)[C@@H](O)[C@@]1(C)F]

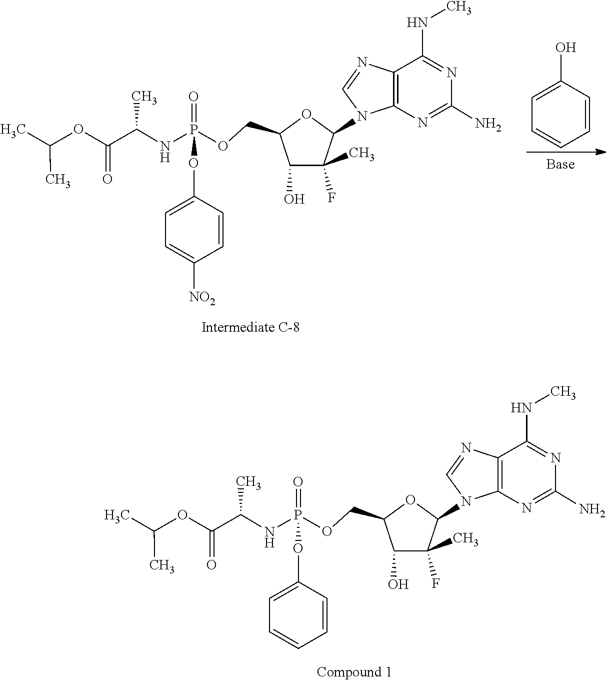 [SMILES: CNc1nc(N)nc2c1ncn2[C@@H]1O[C@H](CO[P@@](=O)(N[C@@H](C)C(=O)OC(C)C)Oc2ccc([N+](=O)[O-])cc2)[C@@H](O)[C@@]1(C)F ; Oc1ccccc1 ; CNc1nc(N)nc2c1ncn2[C@@H]1O[C@H](CO[P@](=O)(N[C@@H](C)C(=O)OC(C)C)Oc2ccccc2)[C@@H](O)[C@@]1(C)F]

TABLE 23
Leaving group comparison

| | | | | | | | | | Temp. | | | Compound 1 |
|---|---|---|---|---|---|---|---|---|---|---|---|---|
| | Starting | | | | | | | | (° C.)/ | | | HPLC Area |
| Intermediate | Material | | Phenol | | Guanidine | | Solvent | | Time | | P_{R}/P_{S} | Percent |
| C-1 | 0.3 | g | 0.84 | g | 0.23 | g | DMSO | | 20-30/21 | h | 19.2/1 | 80.63 |
| | 1.0 | eq. | 18.0 | eq. | 3.0 | eq | 6 | mL | | | | |
| C-2 | 0.50 | g | 0.92 | g | 0.28 | g | DMSO | | 20-30/8 | h | 9.8/1 | 53.15 |
| | 1.0 | eq. | 12.0 | eq. | 3.0 | eq | 5 | mL | | | | |
| C-3 | 0.5 | g | 0.94 | g | 0.29 | g | DMSO | | 20-30/21 | h | 12.5/1 | 73.76 |
| | 1.0 | eq. | 12.0 | eq. | 3.0 | eq | 10 | mL | | | | |
| C-4 | 0.3 | g | 0.27 | g | 0.34 | g | DMSO | | 20-30° | C. | 11.8/1 | 55.95 |
| | 1.0 | eq. | 6.0 | eq. | 6.0 | eq | 6 | mL | 8 | h | | |
| C-5 | 1 | g | 2.79 | g | 0.57 | g | DMSO | | 20-30/4 | h | 16.1/1 | 85.31 |
| | 1.0 | eq. | 18.0 | eq. | 3.0 | eq | 10 | mL | | | | |
| C-6 | 0.25 | g | 0.69 | g | 0.14 | g | DMSO | | 20-30/21 | h | 13.5/1 | 80.13 |
| | 1.0 | eq. | 18.0 | eq. | 3.0 | eq | 2.5 | mL | | | | |

TABLE 24
Stoichiometry Study with Guanidine

| | | | | | | | | | | | Compound 1 |
|---|---|---|---|---|---|---|---|---|---|---|---|
| | | | | | | | | Temp. | | | HPLC Area |
| C-1 | | Phenol | | Guanidine | | Solvent | | (° C.)/Time | | P_{R}/P_{S} | Percent |
| 0.3 | g | 0.28 | g | 0.35 | g | DMSO | | 20-30/8 | h | 10.5/1 | 70.06 |
| 1.0 | eq. | 6.0 | eq. | 6.0 | eq | 6 | mL | | | | |
| 0.3 | g | 0.28 | g | 0.35 | g | DMSO | | 20-30/21 | h | 5.9/1 | 65.53 |
| 1.0 | eq. | 6.0 | eq. | 6.0 | eq | 6 | mL | | | | |
| 0.3 | g | 0.56 | g | 0.23 | g | DMSO | | 20-30/8 | h | 28.7/1 | 69.86 |
| 1.0 | eq. | 12.0 | eq. | 3.0 | eq | 6 | mL | | | | |
| 0.3 | g | 0.56 | g | 0.23 | g | DMSO | | 20-30/21 | h | 13.4/1 | 79.97 |
| 1.0 | eq. | 12.0 | eq. | 3.0 | eq | 6 | mL | | | | |
| 0.3 | g | 0.84 | g | 0.23 | g | DMSO | | 20-30/8 | h | 43.7/1 | 64.26 |
| 1.0 | eq. | 18.0 | eq. | 3.0 | eq | 6 | mL | | | | |
| 0.3 | g | 0.84 | g | 0.23 | g | DMSO | | 20-30/21 | h | 19.2/1 | 80.63 |
| 1.0 | eq. | 18.0 | eq. | 3.0 | eq | 6 | mL | | | | |

TABLE 25
Stoichiometry study with tetramethyl guanidine

| | | | Solvent | | | | |
|---|---|---|---|---|---|---|---|
| | Tetramethyl | | Volumes | | | | Compound 1 |
| Phenol | guanidine | Temperature | (1:1 | | | | HPLC Area |
| Equivalents | equivalents | (° C.) | DMSO/ACN) | Time | | P_{R}/P_{S} | Percent |
| 22 | 2.25 | 30 | 10 | 6.5 | h | 31.4/1 | 69.42 |
| | | | | 21 | h | 13.2/1 | 80.11 |
| 15 | 3.31 | 30 | 10 | 6.5 | h | 10.1/1 | 77.64 |
| | | | | 21 | h | 4.64/1 | 68.32 |
| 15 | 2.25 | 30 | 10 | 6.5 | h | 16.2/1 | 76.98 |
| | | | | 21 | h | 7.67/1 | 75.25 |
| 15 | 2.25 | 30 | 10 | 6.5 | h | 18.9/1 | 76.27 |
| | | | | 21 | h | 8.06/1 | 75.69 |
| 15 | 2.25 | 30 | 10 | 6.5 | h | 20.6/1 | 74.10 |
| | | | | 21 | h | 8.72/1 | 76.82 |
| 15 | 1.19 | 30 | 10 | 6.5 | h | 41.2/1 | 56.42 |
| | | | | 21 | h | 9.06/1 | 74.41 |
| 15 | 2.25 | 30 | 10 | 6.5 | h | 24.2/1 | 72.31 |
| | | | | 21 | h | 9.98/1 | 77.88 |
| 15 | 2.25 | 30 | 10 | 6.5 | h | 23.4/1 | 73.08 |
| | | | | 21 | h | 9.85/1 | 77.42 |
| 20 | 3 | 30 | 10 | 6.5 | h | 29.1/1 | 73.07 |
| | | | | 21 | h | 12.3/1 | 80.22 |
| 8 | 2.25 | 30 | 10 | 6.5 | h | 10.1/1 | 73.14 |
| | | | | 21 | h | 5.4/1 | 66.03 |
| 10 | 3 | 30 | 10 | 6.5 | h | 9.3/1 | 74.00 |
| | | | | 21 | h | 4.82/1 | 65.65 |
| 20 | 1.5 | 30 | 10 | 6.5 | h | 72.3/1 | 46.29 |
| | | | | 21 | h | 29.8/1 | 66.91 |
| 10 | 1.5 | 30 | 10 | 6.5 | h | 15.1/1 | 70.87 |
| | | | | 21 | h | 8.68/1 | 73.11 |

### Example 38. Synthesis of Compound 1

Procedure for synthesis of Intermediate A-1

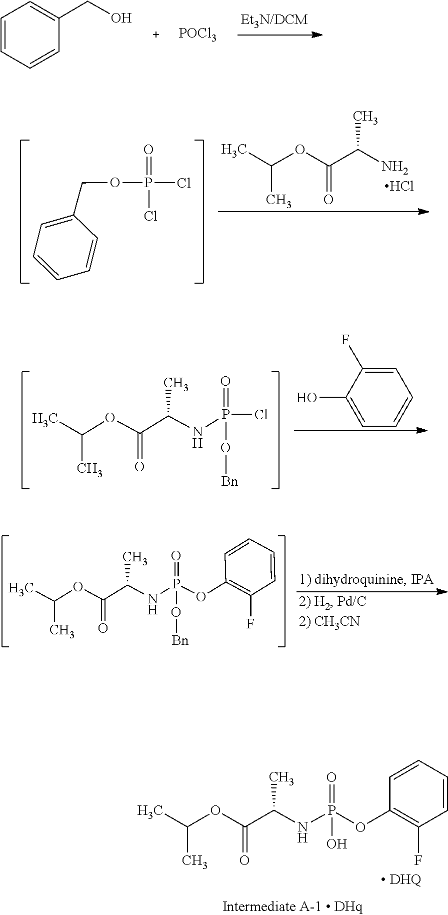

Dichloromethane (2.0 L, 10 vol) was charged to a three-necked flask at 10° C. The flask was evacuated and refilled with nitrogen gas three times. Phosphorous oxychloride (200 g, 1.0 equiv) was charged to the flask, before the mixture was cooled to −55° C. Dichloromethane (200 mL, 1 vol), benzyl alcohol (141 g, 1 equiv), and triethylamine (139 g, 1.05 equiv) were added to a mixing flask at 10° C., before charging the resultant mixture to the phosphorous oxychloride solution over 40 minutes under nitrogen protection. The reaction mixture was stirred for 1 hour at −55° C., during which time white solids precipitated. The reaction appeared to be 99.6% completed, by HPLC analysis of a reaction mixture sample derivatized with benzylamine. L-Alanine isopropyl ester hydrochloride (219 g, 1 equiv) was charged to the reaction mixture, followed by triethylamine (271 g, 2.05 equiv) over 30 minutes.

The resultant reaction mixture was stirred for 1-2 hours, then analyzed for consumption of the first intermediate by HPLC analysis of a sample derivatized with benzylamine. The reaction appeared to be 97.7% complete.

Dichloromethane (400 mL, 2 vol), 2-fluorophenol (146 g, 1 equiv), and triethylamine (139 g, 1.05 eq, added over 20 minutes) were combined in a mixing flask at 10° C., before addition to the reaction mixture over 43 minutes. The batch was warmed from −55° C. to 10° C. over a period of 3 hours, then stirred 2-4 hours. HPLC analysis of a sample derivatized with benzylamine showed 2.0% area of the second intermediate. The batch was filtered, and the filter cake was rinsed with dichloromethane (400 mL, 2 vol). Water (600 mL, 3 vol) was charged to the filtrate, and the mixture was stirred at 10° C. for 5 minutes. The mixture was allowed to settle, and the layers were separated. Hydrochloric acid in water (3.7% w/w, 1 L, 5 vol) was charged to the organics and the mixture was stirred for 5 minutes at 10° C. The mixture was allowed to settle, and the layers were separated. Sodium bicarbonate in water (5% w/w, 1 L, 5 vol) was charged to the organics and the mixture was stirred for 5 minutes at 5° C. The mixture was allowed to settle, and the layers were separated. Water (600 mL, 3 vol) was charged to the organics, and the mixture was stirred at 10° C. for 5 minutes. The mixture was allowed to settle, and the layers were separated. Charcoal (10 g, 5% w/w) was charged to the organics, and the resulting suspension was stirred for 2 hours at 10° C. The suspension was filtered to remove the charcoal, and the filter was rinsed with dichloromethane (100 mL). The filtrate was concentrated under vacuum at 45° C. to a volume of about 600 mL (3 vol). Isopropanol (600 mL, 3 vol) was charged to the mixture, which was again concentrated to about 600 mL (3 vol). Isopropanol (600 mL, 3 vol) was charged to the mixture, the mixture was adjusted to 25° C., and a sample was taken to quantify the amount of dichloromethane remaining (0.25% remained).

Isopropanol (1.9 L, 9.5 vol) was charged to the mixture, followed by dihydroquinine (DHQ, 387 g, 0.908 equiv). The mixture was stirred until a clear solution was formed. Palladium on carbon (10% w/w, KF=63%, 13.7 g) was charged to the mixture. The resulting suspension was degassed with nitrogen twice, then degassed with hydrogen three times. The mixture was stirred for 18-20 hours at 25° C. under 1 atm of hydrogen gas. Analysis of a sample by HPLC showed 0.05% of the third intermediate remaining.

The suspension was filtered and the cake was rinsed with isopropanol (100 mL, 0.5 vol). Solvent was distilled form the mixture under vacuum at 55° C. to a volume of approximately 1200 mL (6 vol), then acetonitrile (1.5 L, 7.5 vol) was charged to the mixture to provide a suspension. The distillation and acetonitrile addition were repeated two additional times. A sample of the resultant suspension was diluted with N-methyl pyrrolidone and analyzed to check for residual isopropanol (0.7% found). The suspension was stirred at 80-90° C. for 1-2 hours to provide a clear solution. The mixture was cooled to 5° C. with a cooling rate of 20° C. per hour, then stirred for 2-3 hours. The suspension was filtered and the cake was washed with cold acetonitrile (400 mL, 2 vol, at 5° C.). The cake was dried at 55° C. for 18-20 hours without vacuum to provide 591.7 g of Intermediate A-1 as a white powder (97.9% AUC purity, 71.8% yield over 4 steps).

### Characterization Data for Intermediate A-1:

¹H NMR (400 MHz, DMSO) δ 12.50 (s, 1H), 8.75 (d, J=4.5 Hz, 1H), 7.96 (d, J=9.2 Hz, 1H), 7.70-7.48 (m, 3H), 7.41 (dd, J=9.2, 2.5 Hz, 1H), 7.12 (dd, J=11.0, 8.2 Hz, 1H), 7.00 (t, J=7.5 Hz, 1H), 6.97-6.86 (m, 1H), 6.59 (s, 1H), 6.05 (s, 1H), 4.77 (dq, J=12.5, 6.2 Hz, 1H), 3.97 (s, 4H), 3.87-3.66 (m, 2H), 3.57-3.34 (m, 2H), 3.07 (s, 1H), 2.84 (d, J=6.4 Hz, 1H), 1.97 (d, J=36.8 Hz, 3H), 1.87-1.61 (m, 2H), 1.38 (t, J=11.8 Hz, 1H), 1.33-1.18 (m, 2H), 1.16 (d, J=6.6 Hz, 3H), 1.09 (dd, J=6.2, 1.2 Hz, 6H), 0.75 (t, J=7.3 Hz, 3H).

¹³C NMR (101 MHz, DMSO) δ 174.69 (d, J=5.9 Hz), 158.27 (s), 154.97 (s), 152.58 (d, J=6.7 Hz), 147.78 (s), 146.04 (s), 144.19 (s), 142.55-142.06 (m), 131.72 (s), 126.16 (s), 124.38 (d, J=3.4 Hz), 122.73 (s), 122.67-122.04 (m), 119.39 (s), 116.16 (d, J=18.9 Hz), 102.09 (s), 67.71 (s), 66.45 (s), 9.32 (s), 56.87 (s), 55.27 (s), 50.96 (s), 43.12 (s), 35.28 (s), 26.04 (s), 24.89 (s), 21.83 (d, J=2.3 Hz), 21.21 (d, J=5.4 Hz), 17.74 (s), 11.86 (s).

³¹P NMR (162 MHz, DMSO) δ 1.04 (s).

¹⁹F NMR (377 MHz, DMSO) δ −132.65 (s).

### Step 2: Coupling Reaction

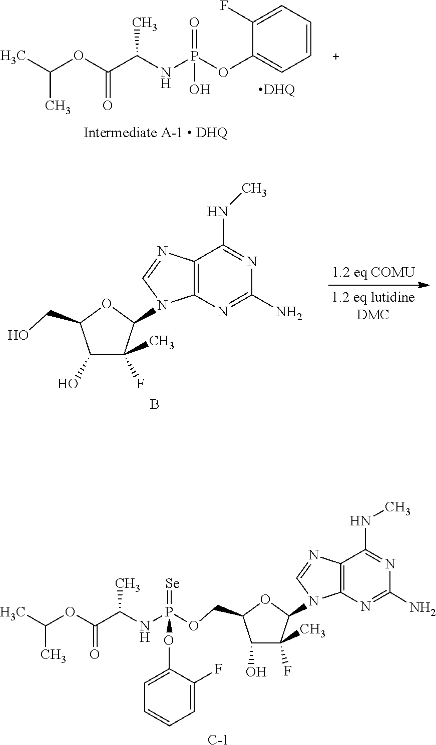

Dichloromethane (1.33 L, 20 vol), Intermediate B (50.0 g, 1.0 equiv, 0.160 mol), dihydroquinine salt of Intermediate A (222 g, 2.2 equiv), and (1-Cyano-2-ethoxy-2-oxoethylidenaminooxy)dimethylamino-morpholino-carbenium hexafluorophosphate (82.3 g, 1.2 equiv) were under nitrogen charged to a 2 μL three-neck flask equipped with a reflux condenser and thermometer.

The resulting suspension was heated to 30-35° C. 2,6-Lutidine (20.6 g, 1.2 equiv) was charged to the reaction mixture over 2 hours and the suspension gradually dissolved during the addition. The mixture was stirred at 30-35° C. for 5-6 hours to provide a clear solution. The reaction was sampled and analysis by HPLC showed less than 5% of Intermediate B remaining. Solution yield calculated by assay of the sample was 64.2 g (66.9% yield). The reaction mixture was cooled to 0-10° C. over 1 hour.

Water (500 mL, 10 vol) was charged to the mixture and the batch was stirred for 2 minutes. Agitation was halted and the batch was allowed to settle into layers before removal of the aqueous layer. Aqueous hydrochloric acid (20% w/w, 133 g, 4.5 equiv) was charged over a period of 1 hour, over which time dihydroquinine salt precipitated. The solution was filtered, and the filter cake was rinsed with dichloromethane (50 mL, 1 vol) and the rinse was combined with the filtrate. Aqueous hydrochloric acid (7% w/w, 167 g, 2.0 equiv) was charged to the filtrate and the batch was agitated for 2 min. Agitation was stopped, the batch was allowed to settle into two layers, and the aqueous layer was removed. The organics were washed with aqueous sodium bicarbonate (5% w/w, 500 g, 1.9 equiv) three times, then washed with water (500 mL, 10 vol). Assay yield of the resultant organic layer was 59.4 g (61.9%). The organic layer was concentrated under vacuum at a temperature of 30-50° C. to a total volume of approximately 200 mL (4 vol). Toluene (500 mL, 10 vol) was charged to the mixture and the mixture was concentrated under vacuum at a temperature of 40-55° C. to a total volume of approximately 250 mL (5 vol). The toluene addition and concentration was repeated one additional time. Ethyl acetate (250 mL, 5 vol) was charged to the batch, and the mixture was heated to 65-75° C. While agitating, the mixture was cooled to 5° C. over 3 hours, during which time crystalline solid formed. The batch was held for 4 hours, then filtered to provide a yellow crystalline solid. Theis solid and ethyl acetate (250 mL, 5 vol) were charged to the flask and heated to 65-75° C. to provide a clear solution. The solution was cooled to 5° C. over 3 hours as crystalline solid formed. The batch was held at 5° C. for 4 hours, then filtered to provide a yellow solid. The yellow solid was dried at 45° C. for 15-16 hours to provide Intermediate C-1 (79 g, 56.2% corrected yield) as a yellow crystalline solid.

### Characterization Data for Intermediate C-3:

¹H NMR (400 MHz, DMSO) δ 7.80 (s, 1H), 7.53-7.40 (m, 1H), 7.40-7.26 (m, 2H), 7.25-7.11 (m, 2H), 6.14 (dd, J=12.9, 10.4 Hz, 1H), 6.11-5.91 (m, 3H), 5.78 (d, J=6.4 Hz, 1H), 4.91-4.71 (m, H), 4.58-4.23 (m), 4.15-4.05 (m, 1H), 3.91-3.72 (m, 1H), 2.88 (s, 3H), 1.32-1.20 (m, 3H), 1.17-0.97 (m, 9H).

¹⁹F NMR (377 MHz, DMSO) δ −131.41 (s), −159.74 (s).

³¹P NMR (162 MHz, DMSO) δ 4.42 (s).

MS (ES+): Expected: 600.2; found: 600.3

### Step 3: Aryloxide Displacement

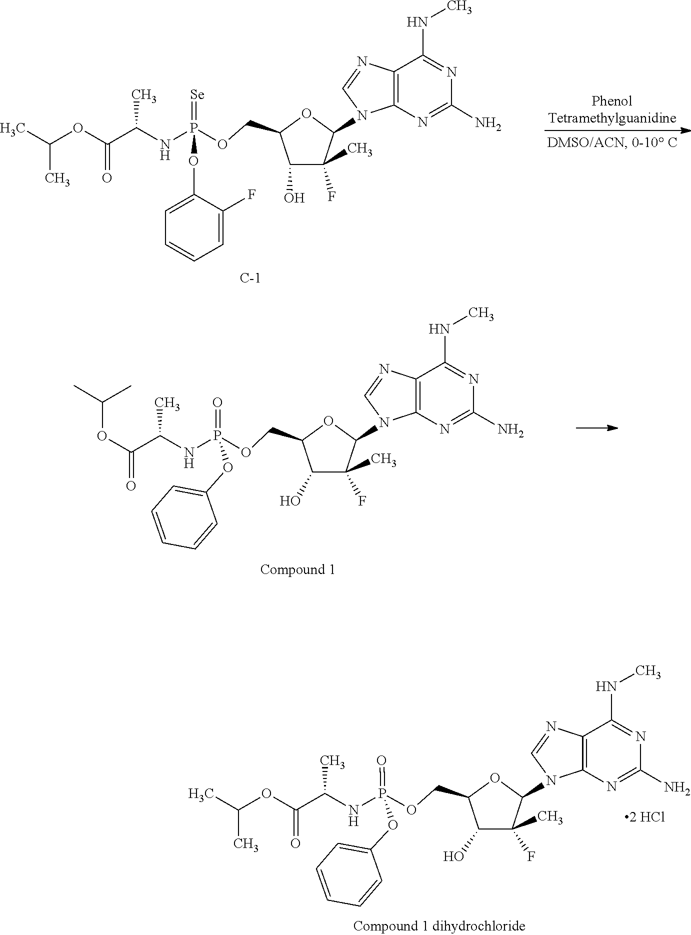

Acetonitrile (255 mL, 8.5 vol), dimethyl sulfoxide (45 mL, 1.5 vol), Intermediate C-1 (30 g, 0.05 mol, 1.0 equiv), phenol (94.1 g, 20.0 equiv) 1,1,3,3-tetramethylguanidine (17.3 g, 3.0 equiv) were charged to a 350 mL flask under nitrogen equipped with a thermometer. The batch was heated to 30° C. for 22 hours while stirring.

The reaction was sampled and remaining Intermediate C-1 was found to be less than 2%. The reaction mixture was cooled to 0-10° C. over 1 hour, and the batch was poured into a reactor containing water (600 mL, 20 vol) while stirring. Methyl-tert-butyl ether (600 mL, 20 vol) was charged to the mixture. Aqueous hydrochloric acid (20% w/w, 90 g, 10 equiv) was charged over 0.5 hours to a pH value of 3-4 while maintaining the temperature of the batch at 0-10° C. The mixture was stirred for an additional 2 minutes, stirring was halted, the aqueous and organic layers were allowed to separate, and the aqueous layer was collected. Water (300 mL, 10 vol) was charged to the organic layer, followed by aqueous hydrochloric acid (20% w/w, 32 g, 3.5 equiv) over 0.5 hours while maintaining the temperature of the batch at 0-10° C.

The mixture was stirred for an additional 2 minutes, stirring was halted, the aqueous and organic layers were allowed to separate, and the aqueous layer was collected. Both aqueous layers were combined and cooled to 0-10° C. Aqueous sodium bicarbonate (5% w/w, 85 g, 1.0 equiv) was added over a period of 0.5 h to bring the pH to 8-9, followed by dichloromethane (300 mL, 10 vol) and water (300 mL, 10 vol). The mixture was stirred for 2 minutes, allowed to settle into two phases, and the aqueous phase was removed. The resultant organics were concentrated at 30-40° C. under vacuum to an approximate volume of 60 mL (2 vol).

Acetone (300 mL, 10 vol) was charged to the mixture. The batch was concentrated at 30-40° C. under vacuum to an approximate volume of 60 mL (2 vol). Acetone (180 mL, 6 vol) was charged to the mixture to provide a clear solution, which was cooled to 0-5° C. Aqueous hydrochloric acid (36.5% w/w, 10 g, 2.0 equiv) was charged in one portion and the mixture was stirred at 0-5° C. for 12 hours. The resultant suspension was filtered, and the recovered solid was dried to provide the bis-hydrochloride salt of Compound 1 (19.2 g, 59% yield) as an off-white solid.

### Characterization Data for the Bis-Hydrochloride Salt of Compound 1:

¹H NMR (400 MHz, DMSO) δ 9.68 (d, J=4.7 Hz, 1H), 8.14 (s, 3H), 7.36 (t, J=7.9 Hz, 2H), 7.18 (dd, J=17.8, 7.9 Hz, 3H), 6.11 (d, J=18.6 Hz, 2H), 4.84 (hept, J=6.2 Hz, 1H), 4.47 (dd, J=10.4, 5.9 Hz, 1H), 4.43-4.2, 1H), 3.77 (s, 1H), 3.15 (d, J=4.8 Hz, 3H), 1.28-1.07 (m, 12H).

¹³C NMR (101 MHz, DMSO) δ 173.14 (d, J=4.3 Hz), 153.46 (s), 151.11 (d, J=6.0 Hz), 149.58 (s), 130.03 (s), 124.98 (s), 120.62 (d, J=4.6 Hz), 112.08 (s), 102.03 (s), 100.24 (s), 80.59 (s), 72.00 (d, J=18.2 Hz), 68.43 (s), 65.85 (s), 50.42 (s), 40.49 (d, J=13.9 Hz), 40.35 (s), 40.14 (s), 39.93 (s), 39.72 (s), 39.51 (s), 39.30 (s), 31.16 (s), 30.07 (s), 21.84 (d, J=7.2 Hz), 20.08 (d, J=7.2 Hz), 17.15 (s), 16.91 (s).

¹⁹F NMR (377 MHz, DMSO) δ −160.09 (s).

³¹P NMR (162 MHz, DMSO) δ 3.66 (s).

MS (ES+): Expected: 582.22; found: 582.35

### General Synthesis of Intermediates C-1 Through C-8

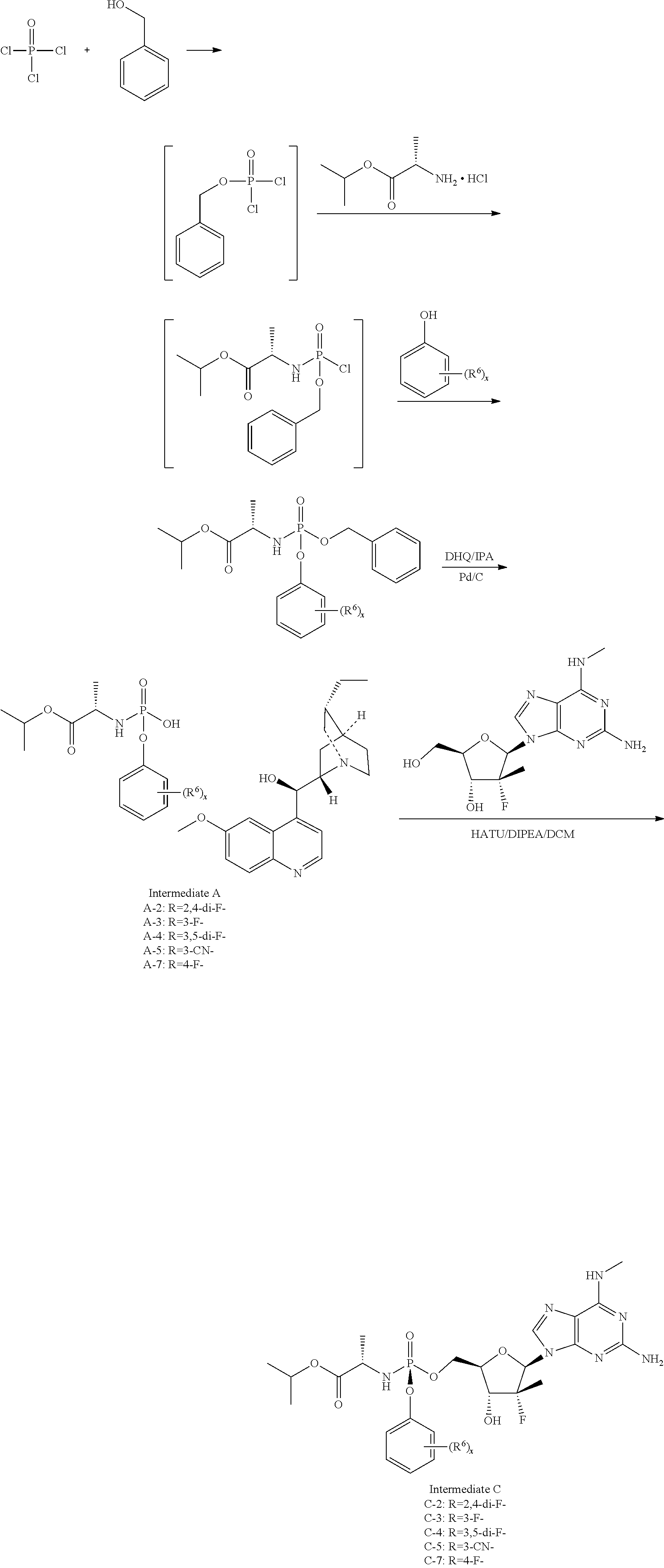

### General Procedure for Intermediate A-2, A-3, and A-7:

A mixture of benzyl alcohol (10.81 g, 0.1 mol) and TEA (10.62 g, 0.105 mol) was added dropwise to a solution of Phosphorus oxychloride (15.3 g, 0.1 mol) in DCM (150 mL) at −60° C. under nitrogen. After addition, the reaction was stirred at −60° C. for 1 h.

L-Alanine isopropyl ester hydrochloride (16.76 g, 0.1 mol) was added to the reaction and then TEA (20.74 g, 0.205 mol) was added dropwise at −60° C. The reaction mixture was stirred at −60° C. for 1 h. A substituted phenol and TEA (10.62 g, 0.105 mol) and DCM (20 mL) was added dropwise to the reaction at −60° C.

After addition, the reaction was stirred to room temperature overnight before being quenched with water. The organic layer was dried over Na₂SO₄, filtered and then the solvent was removed in vacuum. The residue was purified by silica gel column chromatography to give a colorless oil (benzyl protected Intermediates A-2, A-3, and A-7).

The colorless oil (0.1 mol) and isopropanol (200 mL, 5 vol) was charged into a 1000 mL 3-neck glass flask. Dihydroquinine (32.64 g, 0.1 mol) was added and stirred to give a clear solution. To this solution, 5% wet Pd/C (60% water by KF, 5% w/w dry basis) was charged, and hydrogenolysis was performed at 20-25° C. for 18-20 h using 1 atm of hydrogen. When the IPC showed the starting material was consumed completely, the mixture was filtered through a Buchner funnel, and the filtrate was stirred with 4.0 g of charcoal for 2 hours at 25-30° C. The mixture was filtered, and the cake was washed with isopropanol (40 mL). The filtrate was concentrated under vacuum less than 0.09 MPa at 50-60° C. to give a crude product.

Isopropyl acetate (200 mL, 5 vol) was added, and the mixture was concentrated again under vacuum less than 0.09 MPa at 50° C.-60° C. This step was repeated one more time with 5 vol of isopropyl acetate. To the residue, fresh isopropyl acetate (200 ml, 5 vol) was added, and the mixture was stirred at 80° C.-90° C. for 2-3 h to give a clear solution. Then it was cooled to 0° C.-10° C. with a cooling rate of 20° C./1 h and stirred at this temperature for 2-3 h. The solid was collected by filtration, rinsed with cold isopropyl acetate (40 mL, 1 vol) to give the wet cake, which was dried at 50° C. without vacuum for 18 h to give dihydroquinine salt of Intermediate A-2, A-3 or A-7 as an off-white solid.

### Characterization Data for Intermediate A-2, Dihydroquinine Salt:

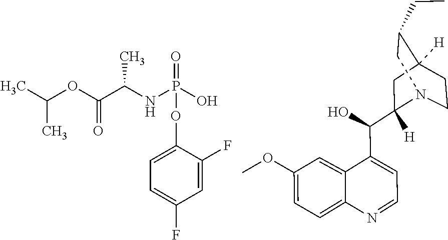

¹H NMR (400 MHz, DMSO) δ 12.30 (s, 1H), 8.74 (d, J=4.5 Hz, 1H), 7.96 (d, J=9.2 Hz, 1H), 7.64 (d, J=4.6 Hz, 1H), 7.62-7.56 (m, 1H), 7.52 (d, J=2.5 Hz, 1H), 7.41 (dd, J=9.2, 2.6 Hz, 1H), 7.25-7.03 (m, 1H), 6.97-6.76 (m, 1H), 6.57 (s, 1H), 6.02 (s, 1H), 4.77 (dt, J=12.5, 6.2 Hz, 1H), 3.97 (s, 4H), 3.75 (dd, J=12.1, 7.7 Hz, 2H), 3.59-3.28 (m, 3H), 3.09 (d, J=5.3 Hz, 1H), 2.86 (s, 1H), 2.01 (s, 2H), 1.92 (s, 1H), 1.88-1.64 (m, 2H), 1.38 (t, J=11.8 Hz, 1H), 1.33-1.18 (m, 2H), 1.14 (d, J=5.6 Hz, 3H), 1.09 (dd, J=6.2, 0.8 Hz, 6H), 0.75 (t, J=7.3 Hz, 3H).

MS (ES−): Expected: 322.07; found: 322.00

### Characterization Data for Intermediate A-3, Dihydroquinine Salt:

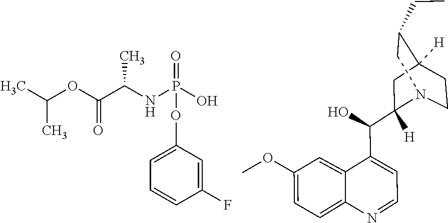

1H NMR (400 MHz, DMSO) δ 12.36 (s, 1H), 8.74 (d, J=4.5 Hz, 1H), 7.96 (d, J=9.2 Hz, 1H), 7.64 (d, J=4.5 Hz, 1H), 7.54 (d, J=2.5 Hz, 1H), 7.41 (dd, J=9.2, 2.6 Hz, 1H), 7.21 (dd, J=15.7, 8.1 Hz, 1H), 7.07 (dd, J=11.5, 2.0 Hz, 1H), 6.94 (d, J=8.2 Hz, 1H), 6.74 (td, J=8.5, 2.4 Hz, 1H), 6.57 (s, 1H), 6.04 (s, 1H), 4.77 (dt, J=12.5, 6.2 Hz, 1H), 3.97 (s, 4H), 3.76 (d, J=5.4 Hz, 2H), 3.63-3.25 (m, 2H), 3.09 (d, J=4.7 Hz, 1H), 2.85 (s, 1H), 1.97 (d, J=34.0 Hz, 3H), 1.88-1.62 (m, 2H), 1.38 (t, J=11.8 Hz, 1H), 1.33-1.18 (m, 2H), 1.18-1.03 (m, 9H), 0.75 (t, J=7.3 Hz, 3H).

MS (ES−) Expected: 304.08; found: 304.04

### Characterization Data for Intermediate A-7, Dihydroquinine Salt:

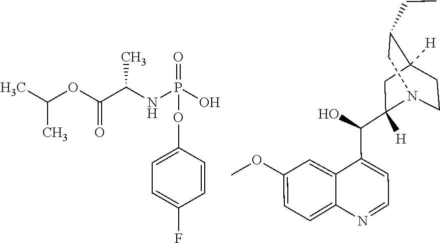

¹H NMR (400 MHz, DMSO) δ 12.56 (s, 1H), 8.74 (d, J=4.5 Hz, 1H), 7.96 (d, J=9.2 Hz, 1H), 7.64 (d, J=4.5 Hz, 1H), 7.54 (d, J=2.6 Hz, 1H), 7.41 (dd, J=9.2, 2.6 Hz, 1H), 7.15 (dt, J=8.4, 4.3 Hz, 2H), 7.09-6.88 (m, 2H), 6.60 (s, 1H), 6.05 (s, 1H), 4.95-4.60 (m, 1H), 3.97 (s, 4H), 3.81-3.58 (m, 2H), 3.47 (d, J=7.4 Hz, 1H), 3.41 (s, 1H), 3.05 (s, 1H), 2.82 (s, 1H), 2.11-1.86 (m, 3H), 1.75 (d, J=36.1 Hz, 2H), 1.28 (ddt, J=20.8, 13.9, 9.0 Hz, 3H), 1.17-1.04 (m, 9H), 0.75 (t, J=7.3 Hz, 3H).

MS (ES−) Expected: 304.08; found: 304.18

### General Procedure for Intermediate A-4 and A-5:

A mixture of benzyl alcohol (10.81 g, 0.1 mol) and TEA (10.62 g, 0.105 mol) was added dropwise to a solution of Phosphorus oxychloride (15.3 g, 0.1 mol) in DCM (150 mL) at −60° C. under nitrogen. After addition, the reaction was stirred at −60° C. for 1 h. L-Alanine isopropyl ester hydrochloride (16.76 g, 0.1 mol) was added to the reaction and then TEA (20.74 g, 0.205 mol) was added dropwise at −60° C. The reaction mixture was stirred at −60° C. for 1 h. The substituted phenol corresponding to the phenyl substitution (0.1 mol) and TEA (10.62 g, 0.105 mol) and DCM (20 mL) was added dropwise to the reaction at −60° C., After addition, the reaction was stirred to room temperature overnight before being quenched with water. The organic layer was dried over Na2SO4, filtered and then the solvent was removed in vacuo. The residue was purified by silica gel column chromatography to give a colorless oil.

The colorless oil (0.1 mol) and Isopropanol (200 mL, 5 vol) was charged into a 1000 mL 3-neck glass flask. Dihydroquinine (32.64 g, 0.1 mol) was added and stirred to give a clear solution. To this solution, 5% wet Pd/C (60% water by KF, 5% w/w dry basis) was charged, and hydrogenolysis was performed at 20° C.-25° C. for 18-20 h using 1 atm of hydrogen. When the IPC showed starting material was consumed completely, the mixture was filtered through a Buchner funnel, and the filtrate was stirred with 4.0 g of charcoal for 2 hours at 25° C.-30° C. The mixture was filtered, and the cake was washed with isopropanol (40 mL).

The filtrate was concentrated under vacuum less than 0.09 MPa at 50° C.-60° C. to give a crude product. Toluene (200 mL, 5 vol) was added, and the mixture was concentrated again under vacuum less than 0.09 MPa at 50° C.-60° C. This step was repeated two more time with 5 vol of Toluene give a residue as an oil. The residue was used the next step without purification.

### Synthetic Procedure for Intermediate A-6:

A mixture of 3-chlorophenol (12.86 g, 0.1 mol) and TEA (10.62 g, 0.105 mol) was added dropwise to a solution of Phosphorus oxychloride (15.3 g, 0.1 mol) in DCM (150 mL) at −60° C. under nitrogen. After addition, the reaction was stirred at −60° C. for 1 h. L-Alanine isopropyl ester hydrochloride (16.76 g, 0.1 mol) was added to the reaction and then TEA (20.74 g, 0.205 mol) was added dropwise at −60° C. The reaction was stirred to room temperature overnight. Filtered and then the solvent was removed in vacuum, MTBE (128 mL, 10 v) was added, and the mixture was stirred for 1 h at 20° C., filtered, and the filtrate was stirred at 0° C. Then citric acid monohydrate (42 g, 0.2 mol) in H₂O (200 mL) was added dropwise to the filtrate at 0° C. The reaction was stirred to room temperature overnight. The reaction was stood for layer separation. The organic layer was stirred with H₂O (100 mL) and then stood for layer separation. DIPEA (12.9 g, 0.1 mol) was added to the organic phase. The mixture was concentrated under vacuum less than 0.09 MPa at 40° C. to give a crude product. The Purified product was obtained by pre-HPLC.

### Characterization Data for Intermediate A-6, Diisopropylethylamine Salt

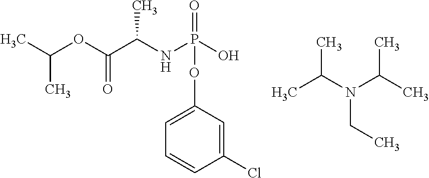

¹H NMR (400 MHz, DMSO) δ 10.25 (s, 1H), 7.23 (dd, J=18.1, 10.0 Hz, 2H), 6.99 (td, J=8.0, 1.3 Hz, 2H), 4.80 (dt, J=12.5, 6.2 Hz, 1H), 3.81-3.62 (m, 2H), 3.60-3.43 (m, 2H), 3.04 (q, J=6.9 Hz, 2H), 2.17-1.95 (m, 1H), 1.32-1.19 (m, 13H), 1.18-1.08 (m, 9H).

MS (ES−): Expected: 320.05; found: 320.10

### Synthetic Procedure for Intermediate A-8

TEA (20.74 g, 0.205 mol) was added dropwise to a solution of 4-nitrophenyl dichlorophosphonate (25.6 g, 0.1 mol) and L-Alanine isopropyl ester hydrochloride (16.76 g, 0.1 mol) in DCM (256 mL, 10 v) at −10° C. under nitrogen. After addition, the reaction was stirred to room temperature overnight. The mixture was concentrated under vacuum less than 0.09 MPa at 40° C. MTBE (256 mL, 10 v) was added, and the mixture was stirred for 1 h at 20° C. Filtered and the filtrate was stirred at 0° C. Tetrabutylammonium Hydroxide (208 g, 0.2 mol, 25% w/w in H2O) was added dropwise to the filtrate at 0° C. After addition, the reaction was stirred to room temperature overnight. The reaction was stood for layer separation. The water layer was stirred with IPAC (200 mL) and then stood for layer separation. The IPAC layer was stirred with aq·HCl (200 mL, 1 mol/L) and then stood for layer separation. The organic layer was dried over Na₂SO₄ and filtered. Dihydroquinine (19.6 g, 0.06 mol) was added to the filtrate. The mixture was concentrated under vacuum less than 0.09 MPa at 50° C. to give a crude product, MTBE (256 mL, 10 v) was added, and the mixture was stirred for 1 h at 20° C. and filtered. The wet cake was dried for 16 h at 60° C. without vacuum. The product Intermediate A-8 was obtained.

### Characterization Data for Intermediate A-8, Dihydroquinine Salt

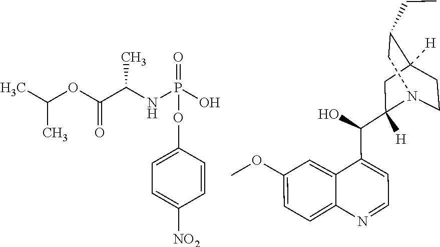

¹H NMR (400 MHz, DMSO) δ 11.89 (s, 1H), 8.74 (d, J=4.5 Hz, 1H), 8.15-8.04 (m, 2H), 7.96 (d, J=9.2 Hz, 1H), 7.64 (d, J=4.4 Hz, 1H), 7.49 (d, J=2.3 Hz, 1H), 7.45-7.32 (m, 3H), 6.59 (s, 1H), 6.00 (s, 1H), 4.75 (dt, J=12.5, 6.2 Hz, 1H), 4.07-3.86 (m, 5H), 3.85-3.68 (m, 1H), 3.62-3.22 (m, 4H), 3.10 (s, 1H), 2.86 (s, 1H), 1.97 (d, J=30.9 Hz, 3H), 1.76 (d, J=35.8 Hz, 2H), 1.40 (t, J=11.7 Hz, 1H), 1.34-1.18 (m, 2H), 1.18-1.00 (m, 9H), 0.75 (t, J=7.3 Hz, 3H).

MS (ES−): Expected: 331.07; found: 331.06

### General Procedure for Intermediates C-1 to C-5 and C-7

The salt of Intermediate A-1 to A-8 (1.7 eq.), Intermediate B (6.25 g, 20 mmol, 1.0 eq), diisopropyl ethylamine (2.6 g, 20 mmol, 1.0 eq.) and HATU (12.9 g, 34 mmol, 1.7 eq.) were added into 188 mL of dichloromethane (30 volumes). The mixture was heated to 40° C. and stirred for 18 hours under nitrogen. The reaction was monitored by TLC and HPLC.

After the reaction was complete, the reaction mixture was cooled to 0-10° C. and was washed with 2N HCl (100 mL) twice, 5% aqueous sodium bicarbonate (100 mL), the separated organic phase was dried over Na₂SO₄ and filtered. The filtrate was concentrated at 40-45° C. under vacuum to give a residue. The residue was purified by silica gel column chromatography to obtain the crude product. The crude product was recrystallized with EA (30 mL), the precipitated was dried without vacuum at 45-50° C. for 16 hours to afford Intermediates C-1 to C-5 or C-7.

### Characterization Data for Intermediate C-2

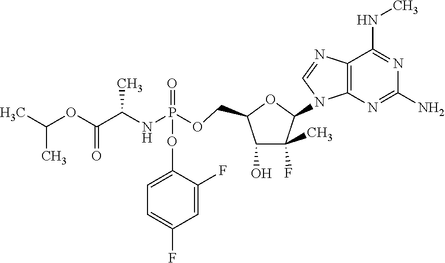 [SMILES: CNc1nc(N)nc2c1ncn2[C@@H]1O[C@H](COP(=O)(N[C@@H](C)C(=O)OC(C)C)Oc2ccc(F)cc2F)[C@@H](O)[C@@]1(C)F]

¹H NMR (400 MHz, DMSO) δ 7.79 (s, 1H), 7.54-7.35 (m, 2H), 7.28 (s, 1H), 7.06 (t, J=8.6 Hz, 1H), 6.27-5.87 (m, 4H), 5.75 (d, J=6.8 Hz, 1H), 4.81 (dt, J=12.5, 6.3 Hz, 1H), 4.37 (ddd, J=18.3, 14.3, 8.5 Hz, 3H), 4.16-3.96 (m, 1H), 3.79 (ddd, J=17.1, 7.2, 2.9 Hz, 1H), 2.88 (s, 3H), 1.24 (d, J=7.1 Hz, 3H), 1.20-1.01 (m, 9H).

MS (ES+): Expected: 618.20; found: 618.24

### Characterization Data for Intermediate C-3:

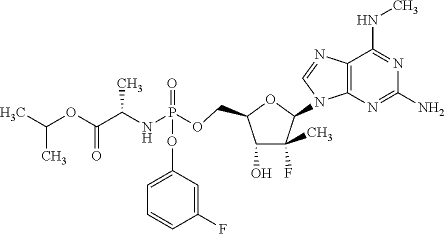 [SMILES: CNc1nc(N)nc2c1ncn2[C@@H]1O[C@H](COP(=O)(N[C@@H](C)C(=O)OC(C)C)Oc2cccc(F)c2)[C@@H](O)[C@@]1(C)F]

¹H NMR (400 MHz, DMSO) δ 7.79 (s, 1H), 7.40 (dd, J=15.3, 8.0 Hz, 1H), 7.27 (s, 1H), 7.16-6.99 (m, 3H), 6.19-5.87 (m, 4H), 5.75 (d, J=6.6 Hz, 1H), 4.81 (dt, J=12.5, 6.2 Hz, 1H), 4.36 (ddd, J=18.4, 14.2, 8.3 Hz, 3H), 4.07 (dd, J=15.2, 8.0 Hz, 1H), 3.93-3.70 (m, 1H), 2.88 (s, 3H), 1.21 (d, J=7.0 Hz, 3H), 1.17-1.02 (m, 9H).

MS (ES+): Expected: 600.21; found: 600.24

### Characterization Data for Intermediate C-4:

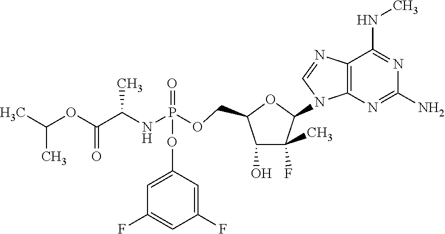 [SMILES: CNc1nc(N)nc2c1ncn2[C@@H]1O[C@H](COP(=O)(N[C@@H](C)C(=O)OC(C)C)Oc2cc(F)cc(F)c2)[C@@H](O)[C@@]1(C)F]

¹H NMR (400 MHz, DMSO) δ 7.79 (s, 1H), 7.28 (s, 1H), 7.15-6.90 (m, 3H), 6.20 (dd, J=12.6, 10.4 Hz, 1H), 6.13-5.88 (m, 3H), 5.77 (d, J=6.3 Hz, 1H), 4.80 (dq, J=12.5, 6.2 Hz, 1H), 4.54-4.24 (m, 3H), 4.07 (dd, J=17.6, 10.7 Hz, 1H), 3.92-3.69 (m, 1H), 2.87 (s, 3H), 1.22 (d, J=7.1 Hz, 3H), 1.15-1.03 (m, 9H).

MS (ES+): Expected: 618.20; found: 618.34

### Characterization Data for Intermediate C-5:

¹H NMR (400 MHz, DMSO) δ 7.79 (s, 1H), 7.75-7.62 (m, 2H), 7.62-7.53 (m, 2H), 7.27 (s, 1H), 6.16 (dd, J=12.7, 10.2 Hz, 1H), 6.10-5.93 (m, 3H), 5.76 (d, J=6.8 Hz, 1H), 4.81 (dt, J=12.5, 6.2 Hz, 1H), 4.53-4.27 (m, 3H), 4.08 (dd, J=13.2, 6.3 Hz, 1H), 3.83 (ddd, J=17.1, 7.2, 2.9 Hz, 1H), 2.88 (s, 3H), 1.23 (d, J=7.1 Hz, 3H), 1.15-1.04 (m, 9H).

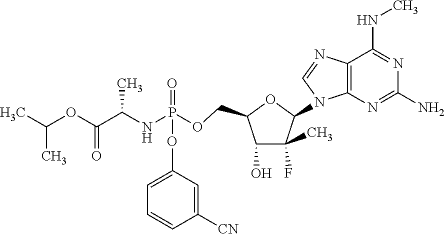 [SMILES: CNc1nc(N)nc2c1ncn2[C@@H]1O[C@H](COP(=O)(N[C@@H](C)C(=O)OC(C)C)Oc2cccc(C#N)c2)[C@@H](O)[C@@]1(C)F]

MS (ES+): Expected: 607.22; found: 607.19

### Characterization Data for Intermediate C-7:

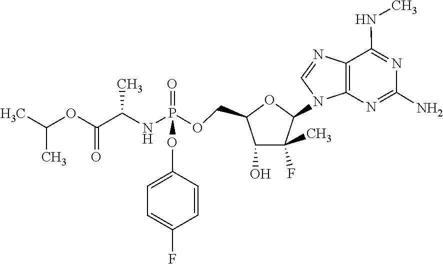 [SMILES: CNc1nc(N)nc2c1ncn2[C@@H]1O[C@H](CO[P@@](=O)(N[C@@H](C)C(=O)OC(C)C)Oc2ccc(F)cc2)[C@@H](O)[C@@]1(C)F]

¹H NMR (400 MHz, DMSO) δ 7.80 (s, 1H), 7.22 (ddd, J=20.8, 13.3, 7.0 Hz, 5H), 6.23-5.86 (m, 4H), 5.73 (d, J=7.1 Hz, 1H), 4.81 (dt, J=12.5, 6.2 Hz, 1H), 4.54-4.20 (m, 3H), 4.06 (dd, J=12.8, 5.8 Hz, 1H), 3.90-3.67 (m, 1H), 2.89 (s, 3H), 1.21 (t, J=6.2 Hz, 3H), 1.15-1.03 (m, 9H).

MS (ES+): Expected: 600.21; found: 600.36

### Synthesis of Intermediate C-6

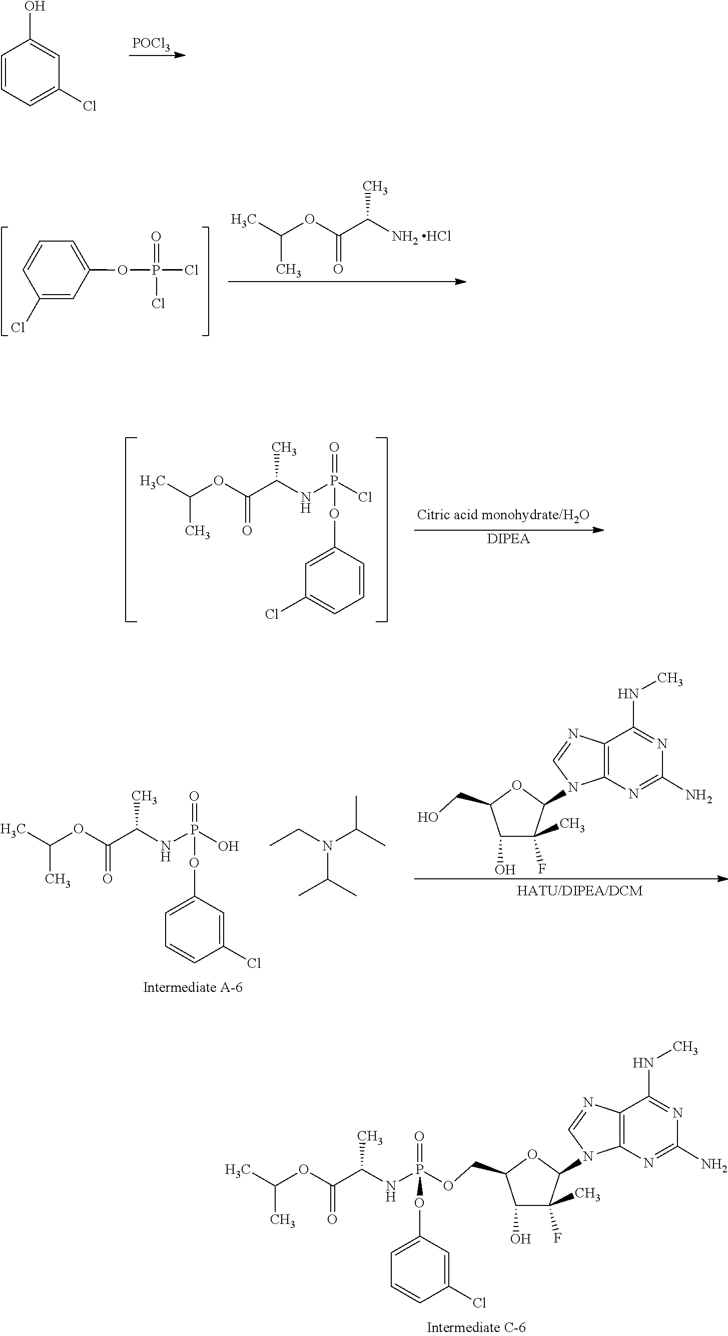

### Characterization Data for Intermediate C-6:

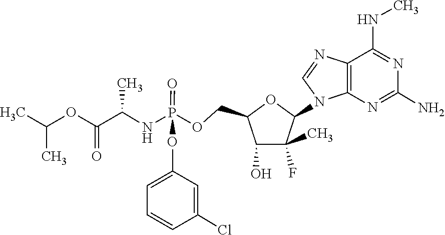 [SMILES: CNc1nc(N)nc2c1ncn2[C@@H]1O[C@H](CO[P@@](=O)(N[C@@H](C)C(=O)OC(C)C)Oc2cccc(Cl)c2)[C@@H](O)[C@@]1(C)F]

¹H NMR (400 MHz, DMSO) δ 7.80 (s, 1H), 7.39 (t, J=8.1 Hz, 1H), 7.33 (s, 1H), 7.26 (d, J=7.9 Hz, 2H), 7.22-7.16 (m, 1H), 6.23-5.89 (m, 4H), 5.76 (d, J=6.8 Hz, 1H), 4.81 (dt, J=12.5, 6.2 Hz, 1H), 4.53-4.23 (m, 3H), 4.16-4.00 (m, 1H), 3.81 (ddd, J=17.1, 7.2, 2.9 Hz, 1H), 2.88 (s, 3H), 1.22 (d, J=7.1 Hz, 3H), 1.17-1.00 (m, 9H).

MS (ES+): Expected: 616.18; found: 616.15

### Synthesis of Intermediate C-8

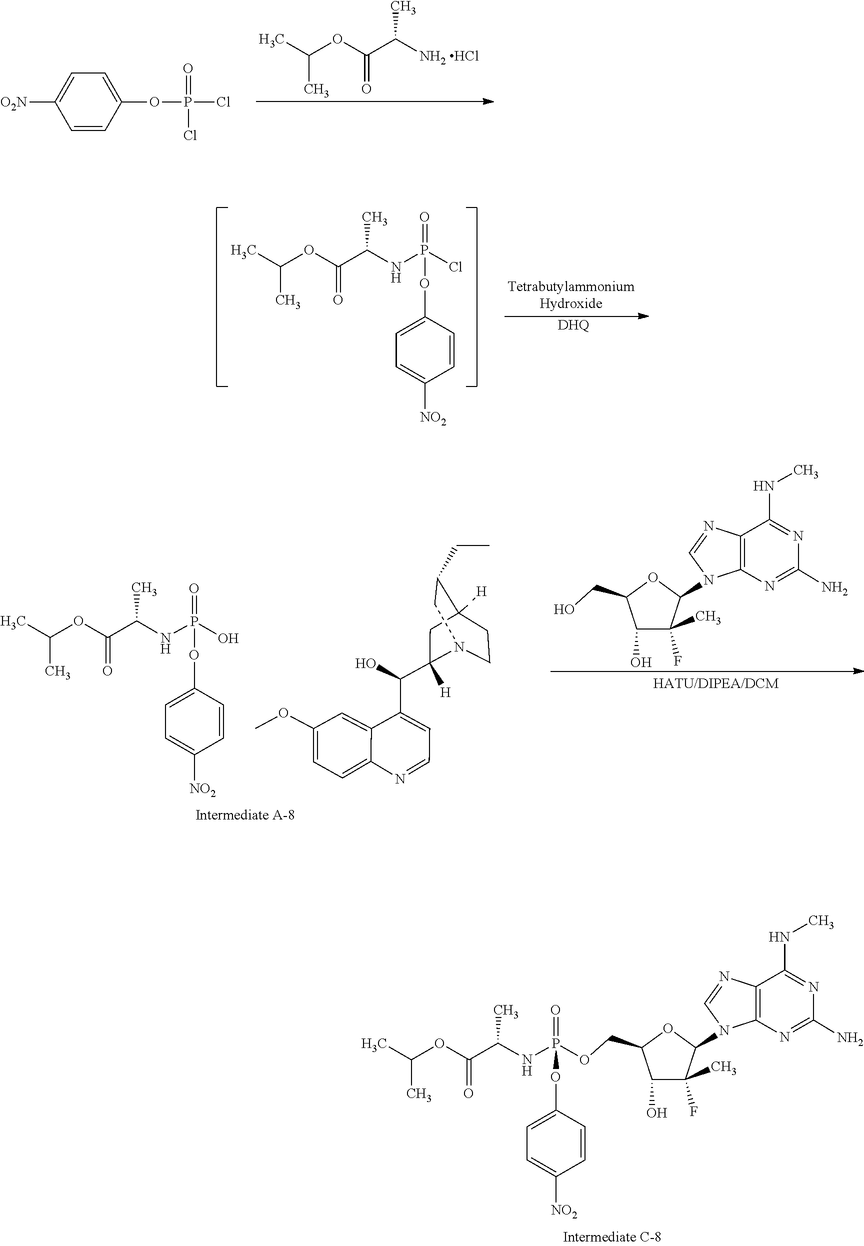

### Characterization Data for Intermediate C-8:

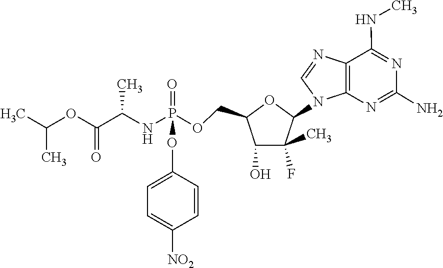 [SMILES: CNc1nc(N)nc2c1ncn2[C@@H]1O[C@H](CO[P@@](=O)(N[C@@H](C)C(=O)OC(C)C)Oc2ccc([N+](=O)[O-])cc2)[C@@H](O)[C@@]1(C)F]

¹H NMR (400 MHz, DMSO) δ 8.31-8.18 (m, 2H), 7.79 (s, 1H), 7.44 (dd, J=14.3, 9.1 Hz, 2H), 7.18 (d, J=46.5 Hz, 1H), 6.35-6.15 (m, 1H), 6.12-5.88 (m, 3H), 5.75 (s, 1H), 4.88-4.73 (m, 1H), 4.45 (dd, J=14.1, 7.1 Hz, 3H), 4.18-4.01 (m, 1H), 3.81 (s, 1H), 2.89 (s, 3H), 1.24 (d, J=7.3 Hz, 3H), 1.10 (dd, J=14.0, 7.5 Hz, 9H).

MS (ES+): Expected: 627.21; found: 627.37

This specification has been described with reference to embodiments of the invention. However, one of ordinary skill in the art appreciates that various modifications and changes can be made without departing from the scope of the invention as set forth in the claims below. Accordingly, the specification is to be regarded in an illustrative rather than a restrictive sense, and all such modifications are intended to be included within the scope of invention.

We claim:

1. A method of treating a Dengue virus infection in a human host in need thereof comprising administering from about 700 mg to about 1,000 mg of a compound of the formula 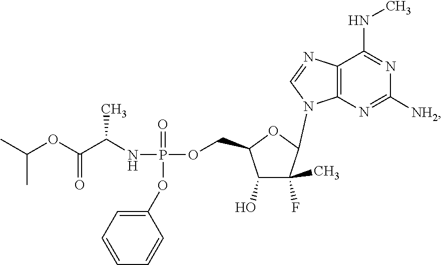 [SMILES: CNc1nc(N)nc2c1ncn2C1O[C@H](COP(=O)(N[C@@H](C)C(=O)OC(C)C)Oc2ccccc2)[C@@H](O)[C@@]1(C)F]
or a pharmaceutically acceptable salt thereof, optionally in a pharmaceutically acceptable carrier, twice, three times or four times per day, wherein the dose refers to the amount of active pharmaceutical ingredient in the dose with the weight added by a pharmaceutically acceptable salt, if present.

2. The method of claim 1, wherein the compound is Compound 1: 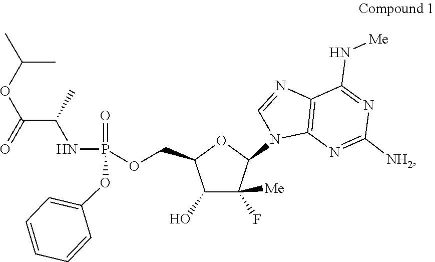 [SMILES: CNc1nc(N)nc2c1ncn2[C@@H]1O[C@H](CO[P@](=O)(N[C@@H](C)C(=O)OC(C)C)Oc2ccccc2)[C@@H](O)[C@@]1(C)F]
or a pharmaceutically acceptable salt thereof.

3. The method of claim 2, wherein the compound is Compound 2:

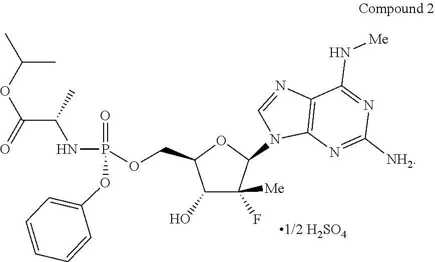 [SMILES: CNc1nc(N)nc2c1ncn2[C@@H]1O[C@H](CO[P@](=O)(N[C@@H](C)C(=O)OC(C)C)Oc2ccccc2)[C@@H](O)[C@@]1(C)F]

4. The method of claim 1, wherein the compound is 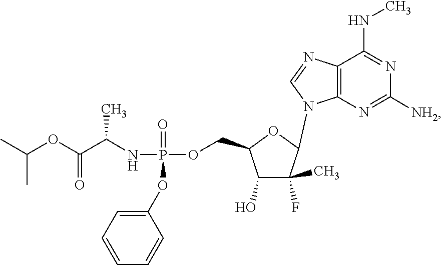 [SMILES: CNc1nc(N)nc2c1ncn2C1O[C@H](CO[P@@](=O)(N[C@@H](C)C(=O)OC(C)C)Oc2ccccc2)[C@@H](O)[C@@]1(C)F]
or a pharmaceutically acceptable salt thereof.

5. A method of treating a Dengue virus infection in a human host in need thereof comprising administering from about 700 mg to about 1,000 mg of a compound of the formula 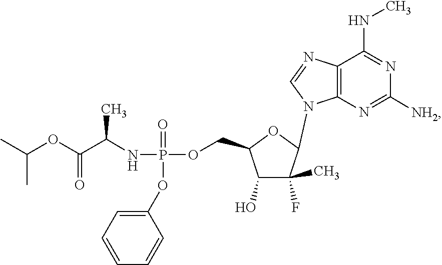 [SMILES: CNc1nc(N)nc2c1ncn2C1O[C@H](COP(=O)(N[C@H](C)C(=O)OC(C)C)Oc2ccccc2)[C@@H](O)[C@@]1(C)F]
or a pharmaceutically acceptable salt thereof, optionally in a pharmaceutically acceptable carrier, twice, three times or four times per day, wherein the dose refers to the amount of active pharmaceutical ingredient in the dose with the weight added by a pharmaceutically acceptable salt, if present.

6. The method of claim 5, wherein the compound is 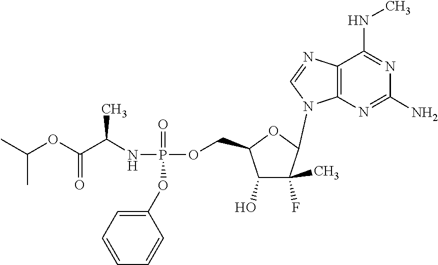 [SMILES: CNc1nc(N)nc2c1ncn2C1O[C@H](CO[P@](=O)(N[C@H](C)C(=O)OC(C)C)Oc2ccccc2)[C@@H](O)[C@@]1(C)F]
or a pharmaceutically acceptable salt thereof.

7. The method of claim 5, wherein the compound is 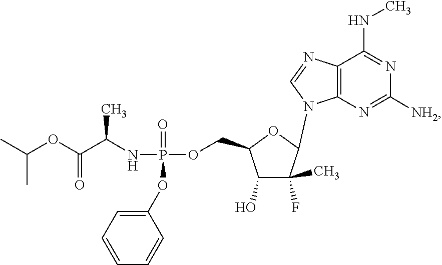 [SMILES: CNc1nc(N)nc2c1ncn2C1O[C@H](CO[P@@](=O)(N[C@H](C)C(=O)OC(C)C)Oc2ccccc2)[C@@H](O)[C@@]1(C)F]
or a pharmaceutically acceptable salt thereof.

8. The method of claim 1, wherein from about 700 mg to about 850 mg of the compound is administered.

9. The method of claim 1, wherein about 750 mg of the compound is administered.

10. The method of claim 1, wherein the compound is administered twice per day.

11. The method of claim 1, wherein the compound is administered three times per day.

12. The method of claim 1, wherein the compound is administered four times per day.

13. The method of claim 1, wherein the compound is administered for four consecutive days.

14. The method of claim 1, wherein the compound is administered for five consecutive days.

15. The method of claim 1, wherein the compound is administered for six consecutive days.

16. The method of claim 1, wherein the human has Dengue virus serotype 1.

17. The method of claim 1, wherein the human has Dengue virus serotype 2.

18. The method of claim 1, wherein the human has Dengue virus serotype 3.

19. The method of claim 1, wherein the human has Dengue virus serotype 4.

20. The method of claim 1, comprising administering 750 mg of Compound 1: 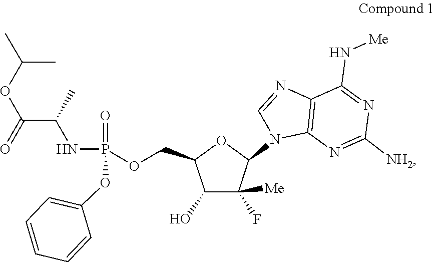 [SMILES: CNc1nc(N)nc2c1ncn2[C@@H]1O[C@H](CO[P@](=O)(N[C@@H](C)C(=O)OC(C)C)Oc2ccccc2)[C@@H](O)[C@@]1(C)F]
or a pharmaceutically acceptable salt thereof, optionally in a pharmaceutically acceptable carrier, three times per day for five days.

21. The method of claim 1, comprising administering 750 mg of a compound of the formula: 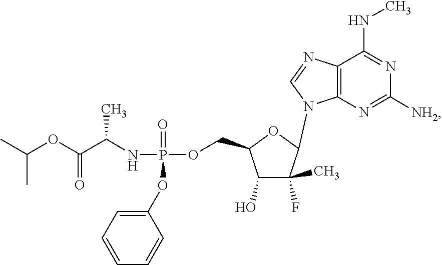 [SMILES: CNc1nc(N)nc2c1ncn2C1O[C@H](CO[P@@](=O)(N[C@@H](C)C(=O)OC(C)C)Oc2ccccc2)[C@@H](O)[C@@]1(C)F]
or a pharmaceutically acceptable salt thereof, optionally in a pharmaceutically acceptable carrier, three times per day for five days.

22. The method of claim 1, comprising administering 750 mg of the hemisulfate salt form of a compound of the formula: 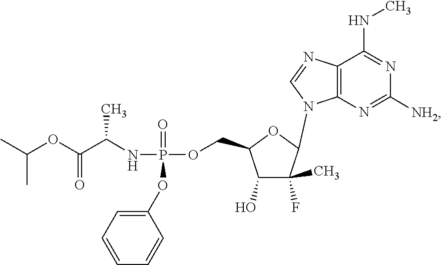 [SMILES: CNc1nc(N)nc2c1ncn2C1O[C@H](CO[P@@](=O)(N[C@@H](C)C(=O)OC(C)C)Oc2ccccc2)[C@@H](O)[C@@]1(C)F]
optionally in a pharmaceutically acceptable carrier, three times per day for five days.

23. A method of treating a Dengue virus infection in a human host in need thereof comprising administering from about 750 mg of a compound of the formula: 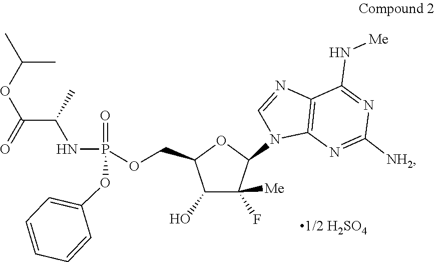 [SMILES: CNc1nc(N)nc2c1ncn2[C@@H]1O[C@H](CO[P@](=O)(N[C@@H](C)C(=O)OC(C)C)Oc2ccccc2)[C@@H](O)[C@@]1(C)F]
optionally in a pharmaceutically acceptable carrier, three times per day for five days.

24. A method of treating a Dengue virus infection in a human host in need thereof comprising administering from about 750 mg of a compound of the formula: 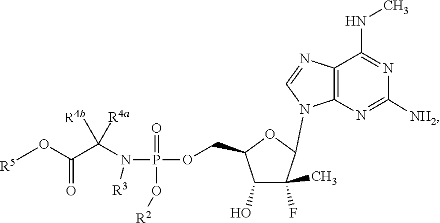
or a pharmaceutically acceptable salt thereof, optionally in a pharmaceutically acceptable carrier, three times per day for five days, wherein
R² is aryl;
R³ is hydrogen;
R^{4a} and R^{4b} are independently selected from hydrogen and C₁₋₆alkyl; and
R⁵ is C₁₋₆alkyl.
